(19) United States
(12) Patent Application Publication
(10) Pub. No.: US 2026/0366013 A1
(43) Pub. Date: Nov. 19, 2026

(54) METHODS AND COMPOSITIONS FOR NUCLEIC ACID LIBRARY AND TEMPLATE PREPARATION FOR DUPLEXED SEQUENCING BY EXPANSION

(71) Applicant: Roche Sequencing Solutions, Inc., Pleasanton, CA (US)

(72) Inventors: Kendall Berg, Seattle, WA (US); Jagadeeswaran Chandrasekar, Seattle, WA (US); Alan Kimura, Seattle, WA (US); Grant Kingsley, Seattle, WA (US); Mark Stamatios Kokoris, Bothell, WA (US); Alexander Lehmann, Seattle, WA (US); John T. Mannion, Palo Alto, CA (US); Lacy McGee, Seattle, WA (US); Robert N. McRuer, Bainbridge Island, WA (US); Melud Nabavi, Seattle, WA (US); McKenna Ann Osentowski, Seattle, WA (US); Marc Prindle, Seattle, WA (US); John C. Tabone, Kirkland, WA (US)

(21) Appl. No.: 19/704,057

(22) Filed: Jun. 10, 2026

Related U.S. Application Data
(63) Continuation of application No. PCT/EP2024/087392, filed on Dec. 19, 2024.
(60) Provisional application No. 63/614,144, filed on Dec. 22, 2023.
(60) Provisional application No. 63/680,104, filed on Aug. 7, 2024.

(51) Int. Cl. C12N 15/10; C12Q 1/6806; C12Q 1/6855

(52) U.S. Cl. CPC C12N 15/1068; C12N 15/1013; C12Q 1/6806; C12Q 1/6855

(57) ABSTRACT

Provided herein are methods for library preparation that may be applied to duplex Sequencing by Expansion. In particular, the present invention relates to methods for generating duplex nucleic acid constructs for use as templates for Xpandomer synthesis and nanopore sequence determination thereof that provide sequence information from both strands of a DNA target fragment in a single run. The present invention also provides methods for epigenetic analysis using duplex template constructs that include a parental strand derived from a library fragment that may include modified nucleobases and a newly synthesized complementary daughter copy strand that includes native nucleobases. The present invention also relates to improved reaction conditions for synthesizing Xpandomer copies of the duplex nucleic acid templates. Compositions and kits for use in the methods are also provided.

### CROSS REFERENCE TO RELATED APPLICATIONS

This patent application is a continuation of International Patent Application No. PCT/EP2024/087392, filed Dec. 19, 2024, which claims priority to and the benefit of U.S. Provisional Application No. 63/614,144, filed Dec. 22, 2023, and U.S. Provisional Application No. 63/680,104, filed Aug. 7, 2024. Each of the above patent applications is incorporated herein by reference as if set forth in its entirety.

### STATEMENT REGARDING SEQUENCE LISTING

The Sequence Listing associated with this application is provided in xml format in lieu of a paper copy, and is hereby incorporated by reference into the specification. The name of the text file containing the Sequence Listing is P39045-US-2_Sequence_Listing. The xml file is 13,312 bytes, was created on Jun. 9, 2026, and is being submitted electronically via Patent Center.

### FIELD OF THE INVENTION

The disclosure relates generally to methods for library preparation that may be applied to duplex Sequencing by Expansion and to methods for epigenetic analysis using duplex template constructs that include a parental strand derived from a library fragment that may include modified nucleobases and a newly synthesized complementary daughter copy strand that includes native nucleobases. Also, disclosure relates to improved reaction conditions for synthesizing Xpandomer copies of the duplex nucleic acid templates, as well as compositions and kits for use in the methods.

### BACKGROUND OF THE INVENTION

Development of nucleic acid sequencing technologies has yielded countless advances in numerous areas. The ability to rapidly and reliably determine the sequence of DNA and RNA molecules has enabled numerous advances in molecular biology, evolutionary biology, medical diagnostics, and molecular medicine, among many other fields.

The accuracy of sequence data that can be reliably obtained when using certain next generation sequencing by synthesis or single molecules nanopore sequencing techniques, however, may be limited due to errors generated during sequencing of the single stranded nucleic acid template molecule. Thus, in many circumstances it can be advantageous to be able to reliably obtain further sequence data of the entire double stranded template molecule. To this end, paired-end, or duplex, sequencing techniques have been employed, e.g., particularly in the context of whole genome shotgun sequencing. Duplex sequencing can allow the determination of two “reads” of sequence from a double stranded nucleic acid target sequence, one from the “sense” strand and one from the “antisense” strand. The knowledge that the paired-end sequences are known to occur on a single duplex, and are therefore linked, or paired, in the genome, can greatly aid assembly of nucleic acid sequences into a consensus sequence, thus greatly improving the accuracy of the sequencing reads. The additional information obtained from paired-end sequencing can also benefit other applications, e.g., applications involving sequencing cell-free DNA such as detection of circulating tumor DNA and prenatal cell-free DNA screening, and various epigenetic detection methodologies.

Provided herein are novel and useful compositions and methods for carrying out paired-end, duplex sequencing. These compositions and methods provide advantages to a number of sequencing methods, e.g., nanopore-based, single molecule sequencing methods.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### BRIEF SUMMARY OF THE INVENTION

The present disclosure provides improved methods, compositions and kits for generating duplex nucleic acid template constructs and their use in duplex sequencing methods, including e.g., Sequencing by Expansion.

In one aspect, the invention provides a method of producing a duplex nucleic acid template, the method including the steps of (a) providing a double stranded nucleic acid target fragment including a first end and a second end; (b) ligating a first adapter to the first end of the double stranded nucleic acid target fragment to produce a sample of first ligation products, in which the first adapter is a hairpin adapter, and in which the strands of the double stranded nucleic acid target fragment are covalently joined by the hairpin adapter; (c) treating the sample of first ligation products with denaturation conditions; and (d) ligating a second adapter to the second end of the double stranded nucleic acid target fragment to produce a sample of second ligation products, wherein the second adapter is a Y adapter. In one embodiment, the method further includes a step of treating the sample of second ligation products of step (d) with an exonuclease enzyme. In other embodiments, the method further includes a step of purifying the second ligation products of step (d). In some embodiments, the molar ratio of the hairpin adapter to the double stranded nucleic acid target fragment is around 3:1 or more. In other embodiments, the molar ratio of the Y adapter to the double stranded nucleic acid target fragment is from around 3:1 to around 10:1 or less. In some embodiments, the hairpin adapter includes a SID. In some embodiments, the Y adapter includes a hybridization site for an extension oligonucleotide. In some embodiments, the denaturing conditions include heat treatment. In some embodiments, the step of purifying the second ligation products from the sample of ligation products of step (d) includes contacting the sample of second ligation products with solid-phase reversable immobilization (SPRI) beads. In some embodiments, the step of ligating a first adapter to the first end of the double stranded nucleic acid target fragment includes incubating the sample for 30 minutes or less under DNA ligation conditions. In other embodiments, the step of ligating a second adapter to the second end of the double stranded nucleic acid target fragment includes incubating the sample for around 30 minutes or less under DNA ligation conditions. In some embodiments, the Y adapter is bound to a solid support. In yet other embodiments, the binding of the Y adapter to the solid support is mediated by a cleavable linker. In some embodiments, the solid support includes streptavidin-coated magnetic beads. In some embodiments, step (b) further includes ligating the first adapter to the second end of the double stranded nucleic acid target fragment to produce the sample of first ligation products. In yet other embodiments, the method further includes the step of fragmenting the sample of first ligation products to produce a sample of fragmentation products, in which the fragmentation products include a first end including the hairpin adapter and a second end lacking the hairpin adapter prior to step (d). In other embodiments, step (d) is combined with step (a) in the same reaction vessel. In yet other embodiments, the ratio of the Y adapter to the double stranded nucleic acid target fragment is 3:1 or more.

In another aspect, the invention provides a method of producing a duplex nucleic acid template, the method include the steps of: (a) providing a double stranded nucleic acid target fragment including a first adapter at a first end and a second adapter at a second end, in which each strand of the double stranded nucleic acid target fragment includes a single stranded break providing a free 3′ end; (b) contacting the double-stranded nucleic acid target fragment with a nucleic acid polymerase under nucleic acid synthesis conditions, in which the nucleic acid polymerase initiates template-dependent synthesis from the free 3′ ends to produce a first and a second nucleic acid synthesis product, in which the first and second nucleic acid synthesis products each include a newly synthesized daughter strand and a free end; and (c) ligating a third adapter to the first and the second nucleic acid synthesis products, in which the third adapter is ligated to the free end of the first and the second nucleic acid synthesis product. In one embodiment, the first adapter and the second adapter are Y adapters. In some embodiments, the free 3′ ends are provided by partial ligation of the first and the second Y adapters to the double stranded nucleic acid target fragment. In some embodiments, the third adapter is a hairpin adapter. In some embodiments, the first adapter and the second adapter are hairpin adapters, wherein the hairpin adapters include a double stranded stem portion and a single stranded loop portion. In yet other embodiments, the free 3′ ends are provided by a single stranded break in one strand of the double stranded stem portion of the hairpin adapters. In some embodiments, the third adapter is a Y adapter. In some embodiments, the nucleic acid synthesis conditions include nucleotide analogs, in which the nucleotide analogs form weaker hydrogen bonds with complementary nucleotides relative to native nucleotides. In yet other embodiments, the nucleotide analogs include N4-Me dCTP and 7-deaza dGTP. In some embodiments, at least one of the adapters includes one or more features selected from the group consisting of a UMI, a SID, an extension oligonucleotide hybridization stie, and a blocking oligonucleotide hybridization site. In some embodiments, the nucleic acid polymerase includes a strand displacing DNA polymerase. In yet other embodiments, the strand displacing DNA polymerase is selected from the group consisting of Klenow fragment, BST large fragment DNA polymerase, and Phi29 DNA polymerase, or variants thereof. In other embodiments, the nucleic acid polymerase includes DPO4 DNA polymerase or a variant thereof. In some embodiments, the duplex nucleic acid template is produced on a solid support. In other embodiments, the duplex nucleic acid template is produced in solution. In some embodiments, the duplex nucleic acid template is treated with an agent that selectively converts native cytosine residues to uracil residues.

In other embodiments, the duplex nucleic acid template is treated with a DNA glycosylase enzyme and an aminoxyalkyl uracil mimetic compound, in which the DNA glycosylase enzyme and the aminoxyalkyl uracil mimetic compound selectively convert epigenetically modified cytosine residues to uracil oxime mimetic residues.

In another aspect, the invention provides a method of producing an opened duplex nucleic acid template, including the steps of: (a) providing a duplex nucleic acid template according to any one of the embodiments disclosed herein; (b) contacting the duplex template construct with an oligonucleotide primer, in which the oligonucleotide primer hybridizes to the hairpin adapter; (c) contacting the oligonucleotide primer with a DNA polymerase under DNA synthesis conditions; and (d) extending the oligonucleotide primer to produce a complementary copy of a first strand of the duplex nucleic acid template and displace a second strand of the duplex nucleic acid template, in which the displaced second strand of the duplex nucleic acid template provides a single stranded template.

In another aspect, the invention provides a methods a method of producing an Xpandomer copy of a nucleic acid template, including the steps of: (a) providing a duplex nucleic acid template according to any of the embodiments disclosed herein; and (b) contacting the duplex nucleic acid template with a modified nucleic acid polymerase under Xpandomer synthesis conditions, in which the Xpandomer copy of the nucleic acid template includes two copies of the nucleic acid target fragment. In some embodiments, the Xpandomer synthesis conditions include one or more of an extension oligonucleotide, a buffer/salt system, a polymerase cofactor, a polymerase enhancing moiety (PEM), XNTP substrates, a phosphate shield molecule, a solvent, a crowding agent, and a blocking oligonucleotide. In some embodiments, the modified nucleic acid polymerase is a variant of DPO4 DNA polymerase (SEQ ID NO:1). In yet other embodiments, the variant of DPO4 DNA polymerase includes an amino acid sequence that is at least 85% identical to SEQ ID NO:2. In some embodiments, the Xpandomer synthesis conditions include an additive as set forth in Table 3. In yet other embodiments, the additive includes a single stranded binding protein (SSB) selected from the group consisting of TTH (SEQ ID NO:3), KOD (SEQ ID NO:4), Gp32 (SEQ ID NO:5), E. coli SSB (SEQ ID NO:6), RPA (SEQ ID NO:7), NCp7 (SEQ ID NO:8), RecA (SEQ ID NO:9), and helicase (SEQ ID NO:10).

In another aspect, the invention provides a method of sequencing a plurality of duplex nucleic acid templates, including the steps of: (a) providing a sample comprising a plurality of duplex nucleic acid templates, in which the plurality of duplex nucleic acid templates each includes a sense strand of a nucleic acid target fragment covalently joined to an antisense strand of a nucleic acid target fragment; (b) generating a copy of the each of the plurality of duplex nucleic acid templates, in which the copies include an Xpandomer, in which the Xpandomers include a sequence of reporter codes, and in which the sequence of reporter codes encodes the sequence of the duplex nucleic acid templates; and (c) determining the sequences of the reporter codes by passing the Xpandomers through a nanopore sensor. In some embodiments, the duplex nucleic acid templates includes the duplex nucleic acid templates according to any one of the embodiments disclosed herein. In some embodiments, the nucleic acid target fragments are isolated from a blood sample. In some embodiments, the time to complete the method is from six to seven hours or less.

In another aspect, the invention provides a composition including a duplex nucleic acid template according to any one of the embodiments disclosed herein.

In another aspect, the invention provides a kit for producing a library of duplex nucleic acid templates, including one or more of: a nucleic acid fragmentation mixture, a first ligation mixture, in which the first ligation mixture includes a hairpin adapter, in which the hairpin adapter includes a SID (HPS), a second ligation mixture, in which the second ligation mixture includes a Y adapter, an exonuclease digest mixture, a nucleic acid purification mixture, and a nucleic acid quantification assay mixture.

In another aspect, the invention provides a method of producing a YSU adapter including the steps of: (a) providing a first sample including a first YSU adapter portion including a 5′ oligonucleotide single stranded arm, a 3′ oligonucleotide single stranded arm and a first double stranded stem portion, in which the first double stranded stem portion includes a SID sequence; (b) providing a second sample including a second YSU adapter portion, including a second double stranded stem portion, in which the second double stranded stem portion includes a UMI sequence; and (c) contacting the first sample and the second sample with a DNA ligase under DNA ligation conditions to produce a YSU adapter comprising a SID sequence and a UMI sequence. In some embodiments, the second sample includes a plurality of second YSU adapter portions, in which each of the plurality of second YSU adapter portions includes a unique UMI sequence.

In another aspect, the invention provides a composition including the YSU adapter according to any of the embodiments described herein.

### BRIEF DESCRIPTION OF THE DRAWINGS

FIGS. 1A, 1B, 1C, 1D, and 1E are condensed schematics summarizing one embodiment of the methods of generating a duplex nucleic template construct of the present invention and use in duplexed Xpandomer synthesis.

FIGS. 2A and 2B are condensed schematics summarizing another embodiment of the methods of generating a duplex nucleic template construct of the present invention utilizing pre-nicked hairpin adapters.

FIGS. 3A, 3B, 3C, 3D, and 3E are condensed schematics summarizing another embodiment of the methods of generating a duplex nucleic template construct of the present invention employing solid-state synthesis and use in duplexed Xpandomer synthesis.

FIG. 4 is a condensed schematic summarizing one embodiment of the methods of generating a duplex parent-parent nucleic template construct of the present invention.

FIG. 5 is a condensed schematic summarizing another embodiment of the methods of generating a duplex parent-parent nucleic template construct of the present invention.

FIG. 6 is a condensed schematic summarizing an embodiment of the methods of opening a duplex nucleic template construct of the present invention prior to Sequencing by Expansion utilizing an invasive oligonucleotide primer to generate a second daughter antisense strand followed by exonuclease-mediated digestion of the second daughter antisense strand.

FIG. 7 is a condensed schematic summarizing another embodiment of the methods of opening a duplex nucleic template construct of the present invention prior to Sequencing by Expansion utilizing an invasive oligonucleotide primer to generate a second daughter antisense strand followed by physical displacement of the second daughter antisense strand via application of a magnetic force.

FIG. 8 is a condensed schematic summarizing one embodiment of using an opened duplex construct of the present invention for target enrichment prior to Xpandomer synthesis.

FIG. 9 is a condensed schematic summarizing another embodiment of using an opened duplex construct of the present invention for target enrichment prior to Xpandomer synthesis.

FIGS. 10A and 10B are condensed schematic summarizing one embodiment of integrating a target enrichment step into the workflow for generating a duplex template construct.

FIGS. 11A and 11B are cartoons illustrating certain embodiments of adapter designs that may be used according to the present invention

FIGS. 12A and 12B are cartoons illustrating certain features of chemo-enzymatic conversion for detection of methylated cytosine nucleobases in a DNA target fragment.

FIG. 13 is a simplified illustration summarizing certain features of an exemplary xNTP substrate used for the synthesis of Xpandomer copies of library template constructs for nanopore sequence determination.

### DETAILED DESCRIPTION OF THE INVENTION

The present invention may be understood more readily by reference to the following detailed description of preferred embodiments of the invention and the Examples included herein. Unless otherwise explained, all technical and scientific terms used herein have the same meaning as commonly understood by one of ordinary skill in the art to which this disclosure belongs.

The practice of the present invention will employ, unless otherwise indicated, conventional techniques of molecular biology, microbiology, recombinant DNA, and so forth which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), the series METHODS IN ENZYMOLOGY (Academic Press, Inc.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987).

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

The abbreviation, “e.g.,” is derived from the Latin exempli gratia, and is used herein to indicate a non-limiting example. Thus, the abbreviation “e.g.,” is synonymous with the term “for example.” It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means 20% of the indicated range, value, or structure, unless otherwise indicated.

### Methods and Compositions for Duplex Sequencing by Expansion

Embodiments described herein may be applied any suitable sequencing platform, including “Next Generation” and nanopore sequencing, but are particularly useful for Sequencing by Expansion (SBX®). Sequencing by Expansion is described in Applicant's published PCT application, WO 2020/236526 A1, “Translocation Control Elements, Reporter Codes, and further means for Translocation Control for use in Nanopore Sequencing,” filed May 14, 2020, and issued patent, U.S. Pat. No. 7,939,259 B2, “High Throughput Nucleic Acid Sequencing by Expansion,” filed Jun. 19, 2008, the entire contents of which are both incorporated herein by reference for all purposes.

In general terms, Sequencing by Expansion (SBX®) uses biochemical polymerization to transcribe the sequence of a DNA template onto a measurable polymer called an “Xpandomer” using highly modified, non-natural nucleotide analog substrates referred to as “XNTPs”. The transcribed sequence is encoded along the Xpandomer backbone in high signal-to-noise reporter codes that are separated by ~10 nm and are designed for high-signal-to-noise, well-differentiated responses. The Xpandomer polymer thus preserves the original genetic information of the target nucleic acid template, while also increasing linear separation of the individual elements of the sequence data. These differences provide significant performance enhancements in sequence read efficiency and accuracy of Xpandomers relative to natural DNA.

Sequencing by Expansion represents a single molecule sequencing technology in that the nanopore detector reads electronic signals from a single Xpandomer molecule at a time. As such, SBX® sequencing reads provide information from a single, contiguous DNA template.

Sequencing by Expansion is discussed in greater detail herein.

The present technology provides improved methods and compositions for duplex (i.e., paired-end) sequencing, which can be applied to both genomic and epigenomic (e.g., methylomic) sequence analysis. Described herein are methods and compositions for generating a library of paired-end, double stranded nucleic acid templates, each including at least a subset of sequence from a target nucleic acid sample. For Sequencing by Expansion applications, the methods and compositions of the present invention allow for sequencing of nucleic acid templates in which both the sense and anti-sense strands (i.e., the two opposite strands of a double stranded nucleic acid target fragment) are copied onto the same Xpandomer molecule for nanopore sequence determination. The present invention also provides library preparation, template preparation, and analysis methods for Sequencing by Expansion, enabled by, e.g., novel adapter designs and synthesis conditions. In some embodiments, the sense and anti-sense strands of the nucleic acid target fragments in a library are separated, and operably joined (i.e., covalently linked), by a known nucleic acid adapter segment.

The general method typically begins with a sample of double stranded nucleic acid fragments having defined ends, which could be blunt ends or ends with known overhang sequences (5′ or 3′ overhangs). These nucleic acid fragments can be of any size or size range and can include DNA, RNA, DNA-RNA hybrids (e.g., molecules produced by first-strand synthesis during cDNA preparation have one mRNA strand and one complementary DNA strand), genomic DNA, cDNA, mRNA, tRNA, etc. In some embodiments, the nucleotide sequence of the fragments is not known.

In some aspects, the invention provides for producing a library of paired-end nucleic acid template constructs from the sample of double stranded nucleic acid fragments for synthesis of Xpandomer copies for nanopore sequence determination. As discussed herein, the terms “paired-end” and “duplex” (or “duplexed”) may be used interchangeably as they relate to template constructs for Xpandomer synthesis. Sequencing of the single, contiguous Xpandomer copies of the paired-end template provides duplexed reads of the original nucleic acid target fragments. The paired-end Xpandomer template constructs can be single nucleic acid chains that each have the following structure: adapter region 1, sense (i.e., forward) nucleic acid strand of the target fragment, adapter region 2, anti-sense (i.e., reverse) nucleic acid strand of the target fragment, adapter regions 3. In some embodiments, adapter region 2 forms a classic “hairpin” structure in which the stem portion of the hairpin adapter is double stranded and is ligated to one end of the double stranded nucleic acid target fragment. The loop portion of the hairpin adapter is single-stranded and operable joins (i.e., covalently links or couples) the sense and antisense strands of the double stranded nucleic acid target fragment. In some embodiments, adapter region 1 and adapter region 3 are derived from a classic “Y adapter” structure, in which the stem portion of the Y adapter is double stranded and is ligated to the opposite end of the double stranded nucleic acid target fragment. The arms of the Y adapter may be single stranded and provide a free 3′ end and a free 5′ end to the paired-end template construct. As described in further detail herein, in several embodiments, the hairpin and Y adapters structures of the present invention may include several novel features that enable synthesis of “daughter strand” copies of the paired-end template constructs useful for, e.g., epigenomic analyses. Certain, non-limiting, examples of alternative library formats and work-flows to enable high accuracy duplex and methylome Sequencing by Expansion are discussed below.

In one aspect, DNA from a biological sample is obtained or provided. The DNA obtained or provided from the biological sample may be genomic DNA, mitochondrial DNA, cell-free DNA (cfDNA), circulating tumor DNA (ctDNA), or a combination thereof.

DNA samples may be obtained from a patient or subject, from an environmental sample, or from an organism of interest. In embodiments, the DNA sample is extracted, purified, or derived from a cell or collection of cells, a body fluid, a tissue sample, an organ, and/or an organelle. In some embodiments, the sample DNA is whole genomic DNA.

In some instances, genomic DNA and mitochondrial DNA may be obtained separately from the same biological sample or source. Many different methods and technologies are available for the isolation of genomic DNA and mitochondrial DNA. In general, such methods involve disruption and lysis of the starting material followed by the removal of proteins and other contaminants and finally recovery of the DNA. Removal of proteins can be achieved, for example, by digestion with proteinase K, followed by salting-out, organic extraction, gradient separation, or binding of the DNA to a solid-phase support (either anion-exchange or silica technology). Mitochondrial DNA may be isolated similarly following initial isolation of mitochondria. DNA may be recovered by precipitation using ethanol or isopropanol. There are also commercial kits available for the isolation of nuclear or mitochondrial DNA. The choice of a method depends on many factors including, for example, the amount of sample, the required quantity and molecular weight of the DNA, the purity required for downstream applications, and the time and expense.

The methods of the present disclosure, in certain embodiments, utilize mild enzymatic and chemical reactions that avoid the substantial degradation associated with methods like bisulfite sequencing. Thus, the methods are useful in analysis of low-input samples, such as circulating cell-free DNA, circulating tumor DNA, and in single-cell analysis.

In some embodiments, the DNA sample is circulating cell-free DNA (cfDNA), which is DNA found in the blood and is not present within a cell. cfDNA can be isolated from blood or plasma using methods known in the art. Commercial kits are available for isolation of cfDNA including, for example, the Circulating DNA Kit (Qiagen). The DNA sample may result from an enrichment step, including, but is not limited to antibody immunoprecipitation, chromatin immunoprecipitation, restriction enzyme digestion-based enrichment, hybridization-based enrichment, or chemical labeling-based enrichment.

In some instances, the isolated DNA is fragmented into a plurality of shorter double stranded DNA target fragments. In general, fragmentation of DNA may be performed physically, or enzymatically.

For example, physical fragmentation may be performed by acoustic shearing, sonication, microwave irradiation, or hydrodynamic shear. Acoustic shearing and sonication are the main physical methods used to shear DNA. For example, the Covaris® instrument (Woburn, MA) is an acoustic device for breaking DNA into 100 bp-5 kb. Another example is the Bioruptor® (Denville, NJ), a sonication device utilized for shearing chromatin, DNA and disrupting tissues. Small volumes of DNA can be sheared to 150 bp-1 kb in length. The Hydroshear® from Digilab (Marlborough, MA) is another example and utilizes hydrodynamic forces to shear DNA. Nebulizers, such as those manufactured by Life Technologies (Grand Island, NY) can also be used to atomize liquid using compressed air, shearing DNA into 100 bp-3 kb fragments in seconds. As nebulization may result in loss of sample, in some instances, it may not be a desirable fragmentation method for limited quantities samples. Sonication and acoustic shearing may be better fragmentation methods for smaller sample volumes because the entire amount of DNA from a sample may be retained more efficiently. Other physical fragmentation devices and methods that are known or developed can also be used.

Various enzymatic methods may also be used to fragment DNA. For example, DNA may be treated with DNase I, or a combination of maltose binding protein (MBP)-T7 Endo I and a non-specific nuclease such as Vibrio vulnificus nuclease (Vvn). The combination of non-specific nuclease and T7 Endo synergistically work to produce non-specific nicks and counter nicks, generating fragments that disassociate 8 nucleotides or less from the nick site. In another example, DNA may be treated with NEBNext® dsDNA Fragmentase® (NEB, Ipswich, MA). NEBNext® dsDNA Fragmentase generates dsDNA breaks in a time-dependent manner to yield 50-1,000 bp DNA fragments depending on reaction time. NEBNext dsDNA Fragmentase contains two enzymes, one randomly generates nicks on dsDNA and the other recognizes the nicked site and cuts the opposite DNA strand across from the nick, producing dsDNA breaks. The resulting DNA fragments contain short overhangs, 5′-phosphates, and 3′-hydroxyl groups. In another example, DNA may be treated with the KAPA EvoPlus V2 kitted reagents available from Roche Sequencing Solutions, Inc. that offers one-step fragmentation and A-tailing reactions.

In some instances, the DNA sample is fragmented into specific size ranges of target fragments. For example, the DNA sample may be fragmented into fragments in the range of about 25-100 bp, about 25-150 bp, about 50-200 bp, about 25-200 bp, about 50-250 bp, about 25-250 bp, about 50-300 bp, about 25-300 bp, about 50-500 bp, about 25-500 bp, about 150-250 bp, about 100-500 bp, about 200-800 bp, about 500-1300 bp, about 750-2500 bp, about 1000-2800 bp, about 500-3000 bp, about 800-5000 bp, or any other size range within these ranges. For example, the DNA sample may be fragmented into fragments of about 50-250 bp. In some instances, the fragments may be larger or smaller by about 25 bp.

In certain embodiments, the fragments are treated to produce blunt ends that are compatible with ligation to a first adapter having a compatible blunt end. Any convenient method for producing blunt ends may be employed, including treatment with one or more enzyme having 5′ and/or 3′ single strand exonuclease activity (e.g., E. coli Exonuclease III) and/or performing a fill-in reaction to extend 3′ recessed ends (e.g., with T4 DNA polymerase). No limitation in this regard is intended.

For epigenetic analysis, in certain embodiments, the DNA target fragments may be any DNA fragment, derived from a biological sample, having a sequence of interest that may or may not include epigenetic modifications or DNA damage to one or more nucleobases. In some aspects, the DNA target fragments may include cytosine modifications (i.e., 5-mC, 5-hmC, 5-fC, 35 and/or 5-caC). The DNA target fragments can be a single DNA molecule in the sample, or may be the entire population of DNA molecules in a sample (or a subset thereof) having, e.g., a cytosine modification. The DNA target fragments can comprise a plurality of DNA sequences such that the methods described herein may be used to generate a library of DNA target fragments that can be analyzed individually (e.g., by determining the sequence of individual targets) or in a group (e.g., by multiplexed DNA sequencing methodologies).

In some embodiments, the methods described herein include the step of adding adapter DNA molecules to double stranded DNA target fragments. An adapter DNA, or DNA linker, is a short, chemically-synthesized, single- or double-stranded oligonucleotide that can be ligated to one or both ends of other DNA molecules. Double-stranded adapters can be synthesized so that each end of the adapter has a blunt end or a 5′ or 3′ overhang (i.e., sticky ends). DNA adapters are ligated to the DNA target fragments to provide sequences for, e.g., primer extension reactions and sequencing reactions with complimentary primers and/or for bioinformatic analysis (e.g., clustering of related sequences into families based on shared unique molecular identifier barcodes, UMIs).

Prior to ligation of adapters, the ends of the DNA fragments can be prepared for ligation. For example, by end repair and creating blunt ends with 5′ phosphate groups. Fragmented DNA may be rendered blunt-ended by a number of methods known to those skilled in the art. In a particular method, the ends of the fragmented DNA are “polished” with T4 DNA polymerase and Klenow polymerase, a procedure well known to skilled practitioners, and then phosphorylated with a polynucleotide kinase enzyme. A single ‘A’ deoxynucleotide is then added to both 3′ ends of the DNA molecules using Taq polymerase or Klenow exo minus polymerase enzyme, producing a one-base 3′ overhang that is complementary to a single base 3′ ‘T’ overhang on the double-stranded end of an adaptor. Terminal deoxythymidines can be added to the oligonucleotides used to assemble the final adapter structure (e.g., s Y adapter or hairpin adapter) during conventional oligonucleotide synthesis processes.

In some instances, the adapters may include two oligonucleotides that are partially complementary such that they hybridize to form a region of double stranded sequence, but also retain a region of single stranded, non-hybridized sequence. The region of single stranded sequence may include “universal” oligonucleotide binding sequences, enabling all target fragments in a library to bind to the same oligonucleotide, which may be a capture oligonucleotide, to localize target fragments to a solid-support, an oligonucleotide primer for a primer extension reaction, a PCR primer, sequencing primer, or combinations thereof. In certain instances, the adapters may include two regions of single-stranded, non-hybridized sequence (i.e., a first, 5′ single stranded region and a second, 3′ single stranded region). This configuration is known in the art as a “Y” adapter. The first and second single stranded regions of a Y adapter are not complementary and may include different primer hybridization sequences and other features.

The portions of the two single stranded regions of the adapters typically include at least 10, or at least 15, or at least 20 consecutive nucleotides on each strand. The lower limit on the length of the single stranded regions will typically be determined by function, for example, the need to provide a suitable sequence for binding of a primer for primer extension, PCR and/or sequencing. Theoretically there is no upper limit on the length of the single stranded regions, except that in general it is advantageous to minimize the overall length of the adapter, for example, in order to facilitate separation of unbound adapters from adapter-ligated double stranded DNA target fragments following the ligation step. Therefore, it is preferred that the single stranded regions should be fewer than 50, or fewer than 40, or fewer than 30, or fewer than 25 consecutive nucleotides in length on each strand.

As used throughout, the term “hairpin adapter” refers to a nucleic acid sequence that has two complementary regions that hybridize to one another to form a double-stranded region with the two complementary regions being connected by a single-stranded loop. The hairpin adapters described herein can be of any length suitable for use in the provided methods. For example, the hairpin adapters can be at least 10, at least 20, at least 30, at least 40, or at least 50, nucleotides in length or longer. Optionally, the hairpin adapters are 15 to 40 base pairs in length.

The double stranded region of the adapter is a short double stranded region, typically comprising 5 or more consecutive base pairs, formed by annealing of the two partially complementary polynucleotide strands. Generally, it is advantageous for the double stranded region to be as short as possible without a loss of function. By “function” in this context is meant that the double stranded regions forms a stable duplex under standard reaction conditions for the enzyme-catalyzed nucleic acid ligation reaction.

The precise nucleotide sequence of the adapters is generally not material to the invention and may be selected by the user such that the desired sequence elements are ultimately included in the common sequences of the library of adapter-ligated double stranded DNA target fragments. Additional sequence elements may be included, for example, to provide binding sites for primers which will ultimately be used in sequencing of complementary copy strands of the DNA target fragments. The adapters may further include “tag” sequences, unique molecular identifiers (UMI), and/or sample identifier sequences, which can be used to tag, track, and differentiate target fragments and complementary copies thereof derived from a particular source. The general features and use of such sequences is well known in the art.

The ends of the single stranded regions of the adapters may be biotinylated or bear another functionalities that enables it to be captured, or immobilized, on a surface, such as a solid support. Alternative functionalities other than biotin are known in the art, e.g., as described in Applicant's published Patent Application no. WO2020/172479 entitled, “Methods and Devices for Solid-Phase Synthesis of Xpandomers for use in Single Molecule Sequencing”, which is herein incorporated by reference in its entirety.

As used herein, the term “contacting” is used in accordance with its plain ordinary meaning and refers to the process of allowing at least two distinct species (e.g., nucleic acid and proteins) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, a nucleic acid protein or enzyme (e.g., a DNA polymerase or ligase).

“Ligation” of adapters to the 5′ and 3′ ends of each fragmented double stranded nucleic acid target fragment involves joining of the two polynucleotide strands of the adapter to the double-stranded target polynucleotide such that covalent linkages are formed between both strands of the two double-stranded molecules. Preferably such covalent linking takes place by formation of a phosphodiester linkage between the two polynucleotide strands but other means of covalent linkage (e.g., non-phosphodiester backbone linkages) may be used. However, it is an essential requirement that the covalent linkages formed in the ligation reactions allow for read-through of a polymerase, such that the resultant construct can be copied in a primer extension reaction using primers which bind to sequences in the regions of the adapter-target construct that are derived from the adapter molecules.

In some instances, the adapters and DNA target fragments may be incubated with a ligase to covalently link the adapters and DNA target fragments. Ligase catalyzes the formation of a phosphodiester bond between juxtaposed 5′ phosphate and 3′ hydroxyl termini in duplex DNA or RNA. The enzyme will join blunt end and cohesive end termini as well as repair single stranded nicks in duplex DNA. An exemplary ligase is T4 ligase, which is the most frequently used enzyme for cloning. Another ligase that may be used is E. coli DNA ligase, which preferentially connects cohesive double-stranded DNA end but is also active on blunt ends DNA in the presence of Ficoll or polyethylene glycol. Another ligase that may be used is DNA ligase Ilia, which is known to function in mitochondria.

The products of the ligation reaction may be subjected to purification steps in order to remove unbound adapter molecules before the adapter-target constructs are processed further.

The ligation of adapters to both free ends of the double stranded DNA target fragments gives rise to a pool of adapter-ligated double stranded DNA target fragments with adapters at the 5′ and 3′ ends of the target.

There are several standard methods for separating the strands of an adapter-ligated double stranded DNA target fragment by denaturation, including thermal denaturation, or chemical denaturation in either 100 mM sodium hydroxide solution or formamide solution. The pH of a solution of single-stranded DNA fragments can be neutralized by adjusting with an appropriate solution of acid, or preferably by buffer-exchange through a size-exclusion chromatography column pre-equilibrated in a buffered solution, including SPRI and others for buffer exchange or size selection. Strands of a double stranded DNA target fragment or adapter-ligated template construct can also be separated by heat treatment, e.g., by incubating the sample of double stranded fragments at around 95 degrees C for around three to five minutes.

As used herein, the term “complementary” refers to nucleic acid sequences that are capable of forming Watson-Crick base-pairs. For example, a complementary sequence of a first sequence is a sequence which is capable of forming Watson-Crick base-pairs with the first sequence. The term “complementary” does not necessarily mean that a sequence is complementary to the full-length of its complementary strand, but the term can mean that the sequence is complementary to a portion thereof. Thus, in some embodiments, complementarity encompasses sequences that are complementary along the entire length of the sequence or a portion thereof. For example, two sequences can be complementary to each other along at least 70%, 75%, 80%, 85%, 90%, 91%, 92%, 93%, 94%, 95%, 96%, 97%, 98%, 99%, or 100% of the length of the sequence. Here, the term “sequence” encompasses, but is not limited to, nucleic acid sequences, polynucleotides, oligonucleotides, probes, primers, primer-specific regions, and target-specific regions. Despite any mismatches, the two sequences should have the ability to selectively hybridize to one another under appropriate conditions.

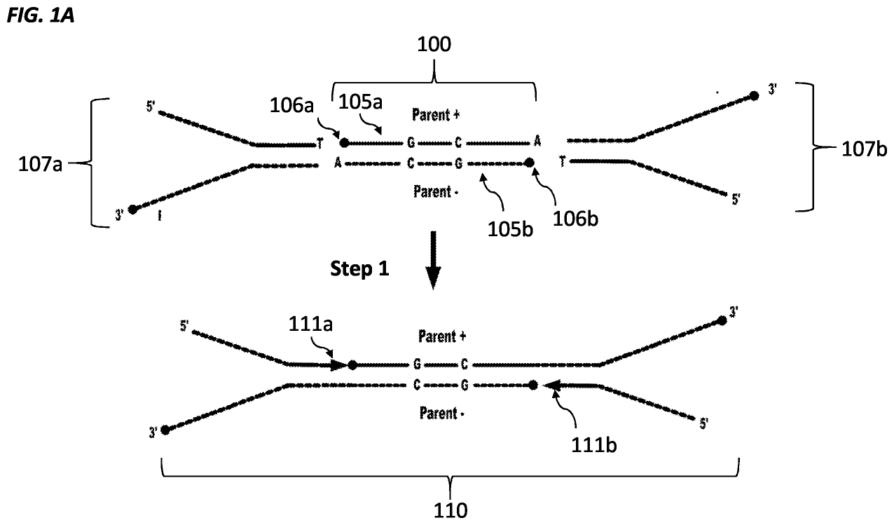

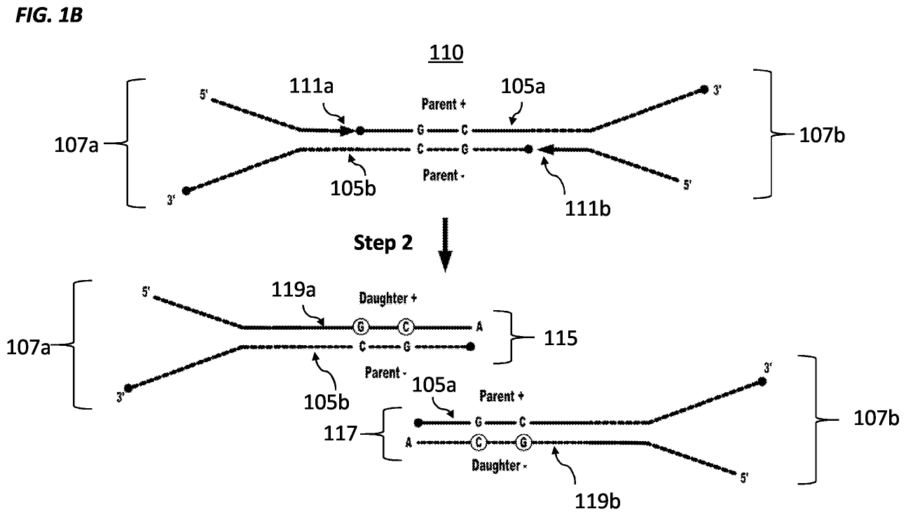

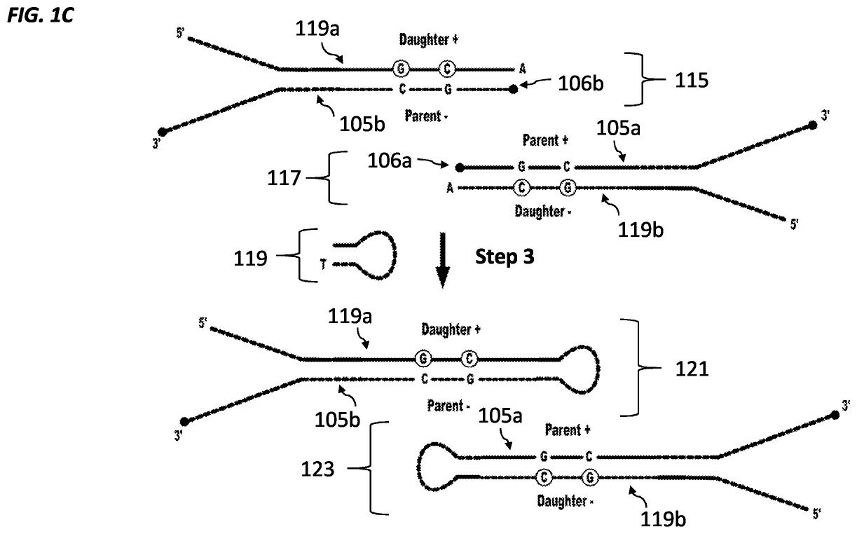

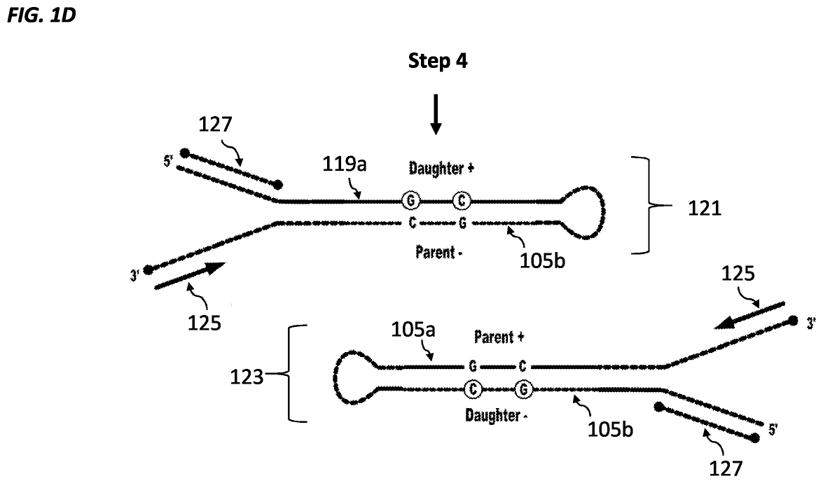

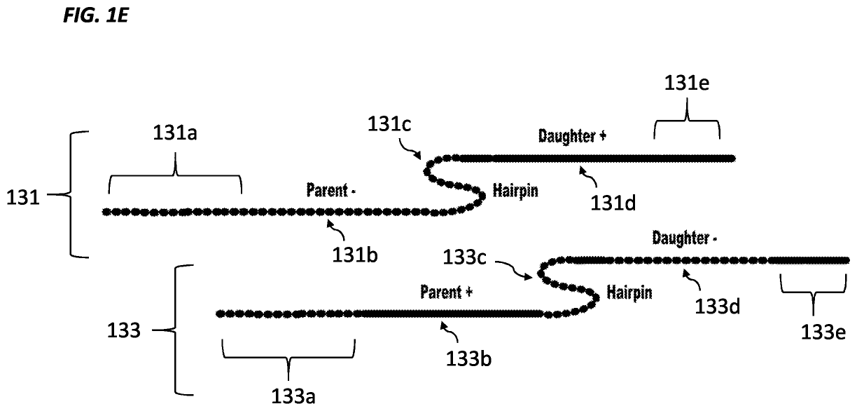

One embodiment of a method of the present invention is set forth in FIG. 1. As shown in FIG. 1A, a double stranded target fragment 100 is derived from genomic DNA or some other nucleic acid source. The double stranded target fragment includes parent (+) strand 105A (i.e., the sense strand) and parent (−) strand 105B (i.e., the antisense strand). In Step 1, adapters 107 and 109 each are joined to an opposite end of the double stranded target fragment. As shown in this embodiment, the adapters are Y adapters and each includes a region of double stranded DNA and two regions of single stranded DNA. In some embodiments, adapters 107 and 109 may have the same overall structure. The adapters are joined to the double stranded target fragment such that one strand of the double stranded region of the adapter is ligated to one strand of the double stranded target fragment, while the other strand of the double stranded region of the adapter is not ligated to the target fragment. In this embodiment, the joining of the adapters to the double stranded target fragment is referred to as “partial ligation”. Partial ligation may be achieved, for example, by blocking the 5′ end of each single strand of the target fragment to prevent ligation to the free 3′ end of the double stranded region of the Y adapter. In one embodiment, blocking is achieved by dephosphorylation of the free 5′ ends of target fragment, as represented by solid circles 106a and 106b. The adapter-ligated double stranded target fragment product 110 includes a single free 3′ end in each strand of the double stranded portion of the ligation product, represented by arrows 111a and 111b at the unligated junctions between the adapter strands and the target fragment strands.

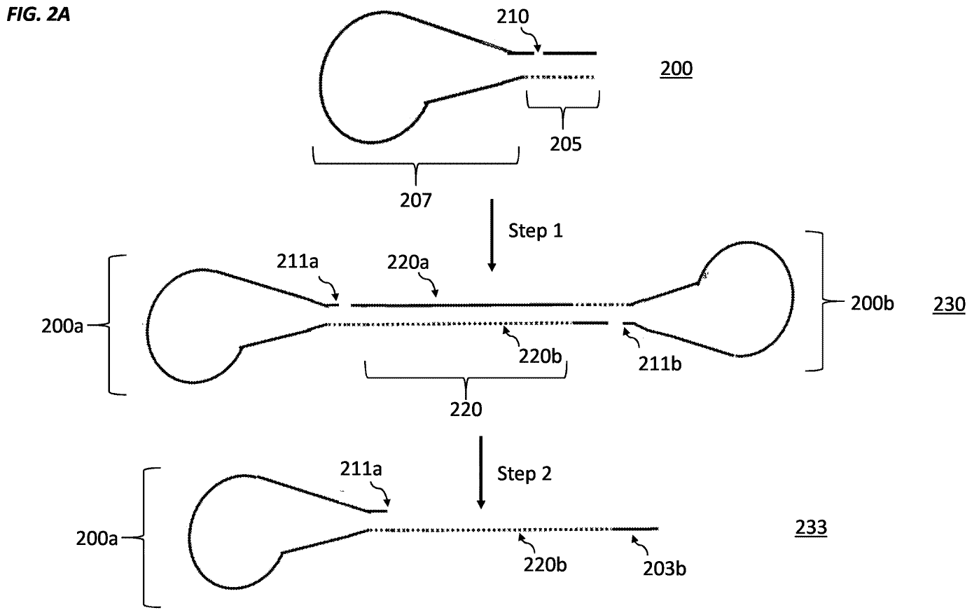

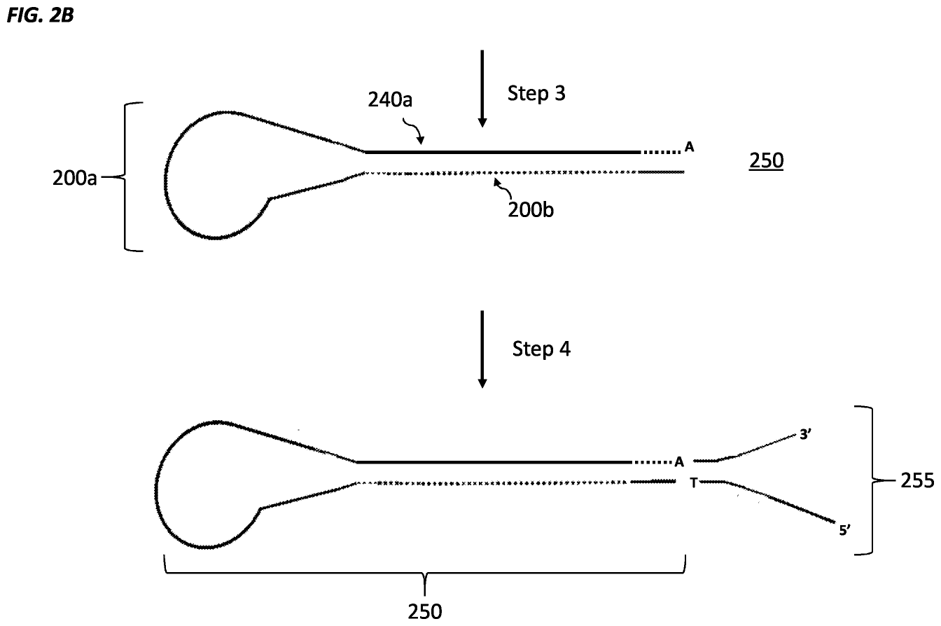

While step 1 in FIG. 1A relies on dephosphorylation of the double-stranded target fragment to produce free 3′ ends 111a and 111b in adapter ligation product 110, it is noted that any method for producing a free 3′ end, single stranded nick, or other site for initiation of nucleic acid synthesis, may be used. For example, Y adapters may be designed that include an internal cleavage site. The adapter-insert ligation product will not include free 3′ ends at the ligation sites between the adapters and insert (i.e., ligation of compatible ends will complete). A free 3′ end may be provided upon treatment of the adapter-insert ligation product with a suitable cleavage agent, For example, in certain embodiments, a nicking endonuclease may be used to create a nick in only one strand of the DNA duplex at a nickase site designed into the Y adapter. As another example, the adapters and the double-stranded target fragments can have ends that are not fully overlapping, thereby leaving a spacer region in one strand upon ligation, which provides a free 3′ end and serve as a site for polymerase binding and nucleic acid synthesis, as described below. It is further noted that the nick/spacer region need not be precisely at the ligation site for the subsequent steps of the method. In other embodiments, a free 3′ end may be created by including a terminal di-deoxoynucleotide in one strand of the double stranded region of the Y adapter. No limitation with respect to free 3′ initiation sites or their location in the adapter-ligated target fragment. As such, any method for obtaining or generating adapter-ligated target fragments of the general structure of ligation product 110 or a similar structure with at least one free 3′ end may be used, such as those discussed with reference to FIGS. 2-5.

In certain embodiments, the adapters may include sequences, or other features, that mediate downstream steps of the workflow. For example, the adapters may include nucleic acid sequences or other chemical moieties for immobilization of the templates on a solid support, sequences for hybridization of oligonucleotide primer(s), sequences enabling bioinformatic analysis of DNA sequence information (e.g., unique molecular identifier bar codes [UMI], sample identifiers [SID]), and the like. In certain embodiments, the structures of adapters 107 and 109 may be identical or different, depending on the particular application. Any of the features discussed herein may be included in either one or both of the adapters, depending on the particular application(s). In certain embodiments, UMI sequences are not required for downstream steps of the duplex sequencing workflow (e.g., bioinformatic error correction or pairing of sequences derived from both strands of a double stranded DNA target fragment). Advantageously, the complexity of adapter design may thus be reduced.

As shown in FIG. 1B, in step 2, the free 3′ ends, 111a and 111b provide initiation sites for a nucleic acid synthesis reaction. Adapter-ligated target fragments 100 are contacted with a strand displacing DNA polymerase, which interacts with free 3′ ends, 111a and 111b. Any suitable strand displacing polymerase may be employed, such as Klenow fragment, BST DNA polymerase large fragment, or Phi29 DNA polymerase, and the like. These polymerases can displace a downstream DNA strand while synthesizing a new strand and generate a long DNA product. When placed under nucleic acid synthesis (i.e., extension) conditions, the DNA polymerase begins nucleic acid synthesis from the free 3′ OH group at sites 111a and 111b using the opposite strand of the target fragment as a template, while simultaneously displacing the complementary strand of the template in the 5′ to 3′ direction.

As the polymerases proceed along the target fragment strands in opposite directions (and on different strands of the target fragment), the two original strands of the target fragment are separated, finally resulting in two nucleic acid extension products 115 and 117. Each of the nucleic acid extension products includes a strand of the original target fragment (e.g., parent strands, 105a and 105b) and a newly synthesized complementary copy stand (e.g., daughter strands 119b and 119a). Each of the nucleic acid extension products includes a fully ligated adapter at one end (i.e., there are no nicks or gaps between the strands of the target fragment and strands of the adapter) and either a blunt or overhang terminus at the opposite end. In nucleic acid extension products, the end opposite the adapter ligated end (i.e., the end lacking an adapter) is expected to be blunt if the polymerase traversed the entire target fragment strand, while it will have a single-stranded 5′ overhang in the parent strand if nucleic acid synthesis terminated before the polymerase reached the end of the target fragment parent strand. Further, if the polymerase employed has terminal transferase activity, there may be a 3′ overhang instead of a blunt end if the polymerase traverses the entire target fragment. No limitation in this regard is intended. In the embodiment depicted in FIG. 1B, the ends of the nucleic acid extension products 115 and 117 include single 3′ A overhangs.

In certain embodiments, the extension reaction may include modified nucleotides that weaken the duplex strength of the extension products, i.e., the strength of the hydrogen bonds between the parent template strand and the newly synthesized daughter strand. Exemplary, non-limiting, modified nucleotides include N4-Me dCTP and 7-deaza dGTP. In embodiment illustrated in FIG. 1B, daughter strands 119a and 119b incorporate such modified dGTP and dCTP analogs, as denoted by the circles around “G” and “C” in these strands.

Under certain conditions, a sample of genomic DNA may sustain genetic damage during one or more steps of the library preparation, e.g., the fragmentation step. For example. 7,8-Dihydro-8-oxoguanine (oxoG) is known in the art to be a predominant oxidative DNA damage lesion that negatively impacts the accuracy of DNA sequencing. For example, replication of parental strands with oxoG lesions with a DNA polymerase often produces errors in the daughter strand as the polymerase misincorporates dATP opposite oxoG, resulting in G to T transversions.

Although high fidelity polymerases extend almost exclusively from an A base opposite an oxoG, it is known that polymerases exist which act in a lesion-induced-error suppressing manner. For example, Y family polymerases, such as DPO4, preferentially extend from a C base opposite an oxoG. Thus, in some embodiments, the DNA synthesis conditions disclosed herein (i.e., the extension reaction) may include a Y family DNA polymerase, such as DPO4, that has the ability to preferentially incorporate the correct dCTP opposite 8-oxo-G, instead of mispairing 8-oxo-G with A.

In some embodiments, the double stranded target fragment can be contacted with reagents that repair other common forms of DNA damage (e.g., abasic sites, nicks, thymidine dimers, blocked 3′ ends, oxidized guanine, oxidized pyrimidines, deaminiated cytosine and the like). For example, a commercially available input genomic DNA cleansing, or repair kit, such as the PreCR® repair mix, (available from NEB) may be included in the DNA synthesis conditions.

As shown in FIG. 1C, in step 3, in one embodiment, 5′ ends 106a and 106b of the parent strands of the extension products may be “activated”, or “unmasked”, by PNK-dependent phosphorylation, thereby enabling ligation. Other means of “masking” (i.e., preventing ligation) and “unmasking” (i.e., enabling ligation) the ends of a nucleic acid strand to either activate or block ligation of two fragments are contemplated by the present invention. For example, chemical masking/unmasking using strategies known in the art may be used in certain embodiments. As further depicted in FIG. 1C, hairpin adapters 119 are contacted with the extension products under DNA ligation conditions and ligated to the double stranded ends to produce first duplexed template construct 121 and second duplexed template construct 123. Ligation of hairpin adapters is facilitated by a single 3′ T overhangs in the hairpin adapters that is capable of base-pairing with the single 3′ A overhang in the daughter stand of the extension products. In certain embodiments, the hairpin adapters may include sequence motifs, or features, that facilitate bioinformatic analysis of downstream sequence data, such as SIDs or any other features discussed herein. In each duplexed template construct, the daughter strand is covalently coupled (i.e., covalently bound) to the parent strand by ligation to intervening hairpin adapter 119.

As shown in FIG. 1D, in step 4, the duplexed template constructs 121 and 123 are prepared for the direct synthesis of Xpandomers. Advantageously, the duplexed template constructs can be directly copied into Xpandomer products without prior PCR amplification. (Although, in certain applications, an amplification step may be included in the methods disclosed herein). This reduces the likelihood of sequencing errors due to nucleotide misincorporations during amplification, particularly in homopolymer sequences in the target fragment. Here, primers 125 are hybridized to sequences in the 3′ ends of the single stranded regions of the Y adapters to provide initiation sites for Xpandomer synthesis. As used herein, primers 125 may be referred to as extension oligonucleotides. In certain embodiments blocker oligonucleotides 127 may be, optionally, hybridized to sequences in the 5′ ends of the single stranded regions of the Y adapters to terminate the Xpandomer synthesis process.

Xpandomer synthesis reactions are carried out in which Xpandomers are synthesized from primers 125 in the 5′ to 3′ direction, as the parent and daughter strands of the template are “unzipped”. In one embodiment, unzipping is facilitated by weak hydrogen bonding between the strands due to modified G and C nucleotide analogs incorporated into the daughter strands, as discussed herein. In other embodiments, any suitable nucleotide analogs may be used to either weaken a base pair, or, alternatively, to strengthen a base pair. For example, in some embodiments, diamino purine (DAP) and/or a suitable analog of thymidine may be used to modify the strength of A:T base pairs. Further details of the Xpandomer synthesis reaction using duplex template constructs are discussed herein.

Full length Xpandomer copies 131 and 133 of duplexed template constructs 121 and 123, respectively, are illustrated in FIG. 1E. From the 5′ to the 3′ direction, each Xpandomer will include copies of the following sequences of the duplexed template construct: a copy of the sequence of the 3′ strand of the Y adapters (131a and 133a, respectively); a copy of the sequence of the parent strand of the template (copy 131b of the parent antisense strand and copy 133b of the parent sense strand, respectively); a copy of the sequence of hairpin adapter (131c and 133c, respectively); a copy of the sequence of the daughter strand copy of the template (copy 131d of the daughter sense strand and copy 133d of the daughter antisense strand, respectively); and a copy of the sequence of the 5′ strand of the Y adapters upstream of the blocker sequence (131e and 133e, respectively). Advantageously, as Xpandomers 131 and 133 are individually passed through a nanopore to obtain sequence information, the resulting sequencing reads will include information from both strands of the original nucleic acid template in a single read, separated by the known sequence of the hairpin adapter that provide a “way point” in bioinformatic sequence analysis. This redundancy in sequence information offers significant advantages in the ability to assess the quality of the read data. In some embodiments, optional inclusion of UMI barcodes in one or more of the adapters provides another level of quality assessment, through the ability to bioinformatically link and compare the sequences of Xpandomer 131 and 133 that each include the sequence of a strand of the original nucleic acid target fragment.

In other embodiments, the methods of the present invention may include alternative adapter designs to generate the duplex template constructs. For example, in one embodiment, an alternative adapter design may include a pre-nicked hairpin adapter, as shown in FIG. 2A. The nick may be introduced into the hairpin adapter according to any suitable method, such as those discussed with reference to FIG. 1A, as well as by using chemical methods known in the art. Here, pre-nicked hairpin adapter 200 includes double stranded region 205 and single stranded loop region 207. The double stranded region 205 includes single stranded nick 210 in one of the strands. In certain embodiments, the sequence of the double stranded region of the adapter may be designed to include a single stranded nickase site. Following synthesis of the adapter, the hairpin may be contacted with a nickase enzyme, followed by a phosphatase enzyme, to generate a single stranded nick in one strand that is refractory to ligation due to the absence of a 5′ phosphate. The single stranded nick therefore provides a free 3′ end capable of being extended by a DNA polymerase. However, the free 3′ end is not capable of being re-ligated to the internal free 5′ end in the hairpin adapter by a DNA ligase. In other embodiments, the single stranded nick may be a gap of one nucleotide or more introduced into the hairpin adapter through deliberate design of two oligonucleotide strands that can be hybridized to form the final hairpin adapter structure. For example, the stem portion of the hairpin adapter can be formed from two oligonucleotide strands that are not completely contiguous, therefore providing a gap with a free 3′ end that provides an initiation site for a DNA polymerase.

As used interchangeably herein, a nicking enzyme, a nickase, or a nicking endonuclease is a protein that binds to double-stranded DNA and cleaves one strand of a double-stranded duplex. The nicking enzyme may cleave either upstream or downstream of the binding site, or nicking enzyme recognition site. When used in connection with a nicking endonuclease, the letters “Nt” or “Nb” indicate whether it is the top (Nt) or bottom (Nb) strand that is cut by the enzyme. In certain embodiments, a suitable nickase will be one that has a relatively long recognition site in order to minimize the occurrence of cleavage at undesired sites in the library fragment.

In certain embodiments, the nicking enzyme may be selected from the group consisting of one or more of the nicking enzymes listed in Table 1. Those of ordinary skill in the art will recognize that various nicking enzymes other than those mentioned specifically herein may be used in the present methods. Nicking enzymes are available from many commercial sources, for example, New England Biolabs (NEB).

TABLE 1
Exemplary Nicking Enzymes

| | Recognition | |
|---|---|---|
| Enzyme | sequence | Isolation source |
| Nb. BbvCI | 3′-GGAGTXCG-5′ | Bacillus brevis strain C |
| Nb. BsmI | 3′-CTTACXGN-5′ | Bacillus stearothermophilus NUB 36 |
| Nt. BstNBI | 5′-GAGTCNNNNVN-3′ | Bacillus stearothermophilus |
| Nt. BspQI | 5′-GCTCTTCNVN-3′ | Bacillus sp. strain Q |
| Nt. BspD6I | 5′-GAGTCNNNNVN-3′ | Bacillus sp. strain D6 |
| Nt. Bst9I | 5′-GAGTCNNNNVN-3′ | Bacillus stearothermophilus strain 9 |
| Nt. BstSEI | 5′-GAGTCNNNNVN-3′ | Bacillus stearothermophilus |
| Nt. BsmAI | 5′-GTCTCNVN-3′ | Bacillus stearothermophilus A664 |
| Nt. AlwI | 5′-GGATCNNNNVN-3′ | Engineered endonuclease |
| Nb. BsrDI | 5′-GCAATGXNN-3′ | Bacillus stearothermophilus D70 |
| Nt. CviPII | 5′-CXCD-3′ | Chlorella virus NYs-1 |
| Nt. CviQII | 5′-RXAG-3′ | Chlorella virus NY-2A |

(note:
D = A or G or T;
N = A or G or C or T;
“X” indicates that the complementary strand of the sequence is recognized and cut;
“V” indicates that the sequence itself is recognized and cut).

In step 1, a double stranded target fragment 220 is provided that is derived from genomic DNA or some other nucleic acid source. The double stranded target fragment 220 includes parent (+) strand 220a (i.e., a sense strand) and parent (−) strand 220b (i.e., an antisense strand). The double stranded target fragment is contacted with pre-nicked hairpin adapters 200a and 200b under DNA ligation conditions. The pre-nicked hairpin are joined (i.e., ligated) to opposite ends of the double stranded target fragment. The adapters are joined to the double stranded target fragment such that each strand of the double stranded region of the adapter is ligated to a strand of the double stranded target fragment. In this embodiment, the joining of the adapters to the double stranded target fragment is referred to as “complete ligation”, in contrast to the partial ligation discussed with reference to FIG. 1A. The adapter-ligated double stranded target fragment product 230 includes a single stranded nick or break of one nucleotide or more in each strand that provides a free 3′ end, represented by arrows 211a and 211b. In addition, because hairpin adapters 200a and 200b are pre-nicked, denaturation of adapter-ligated double stranded target fragment product 230 does not yield a contiguous, covalently joined circle of single stranded DNA.

In certain embodiments, the adapters may include sequences, or other features, that mediate downstream steps of the workflow. For example, the adapters may include nucleic acid sequences or other chemical moieties for immobilization of the templates on a solid support, sequences for hybridization of oligonucleotide primer(s), sequences enabling bioinformatic analysis of DNA sequence information (e.g., unique molecular identifier bar codes [UMI], sample identifiers [SID]), and the like. In certain embodiments, the structures of the adapters may be identical or different, depending on the particular application. Advantageously, in certain embodiments, UMI sequences are not required for downstream steps of the workflow (e.g., bioinformatic error correction or pairing of sequences derived from both strands of a double stranded DNA target fragment) and thus reduce the complexity of adapter design. In some embodiments, the hairpin adapter may include a SID, referred to herein as an “HPS” adapter. The HPS adapter enables, e.g., multiplexing of DNA library samples derived from different sources.

As shown in step 2, adapter-ligated double stranded target fragment 230 is treated with denaturing conditions (e.g., heat or alkaline treatment) to denature the double stranded target fragment and yield two adapter-ligated single stranded target fragments. (In the depiction shown in FIG. 2a, only one of the single adapter-ligated single stranded target fragments is shown for the sake of simplicity). Adapter-ligated single stranded target fragment 233 includes hairpin adapter 200a, single stranded target fragment 220b (here the antisense strand illustrated by a dashed line) and extendable 3′ end 211a. Adapter-ligated single stranded target fragment 233 also includes a single stranded region derived from the stem of hairpin adapter 200b, denoted by solid line 203b.

As shown in FIG. 2B, in step 3, the adapter-ligated single stranded target fragments are contacted with a DNA polymerase under nucleic acid extension conditions, which uses free 3′ end 211a to initiate synthesis of complementary daughter strand 240a, using target fragment strand 220b as a template. Synthesis of a full length complementary daughter strand copy of the target fragment yields adapter-ligated double stranded fragment 250, which includes a strand of the original parental target fragment covalently coupled to a newly synthesized daughter strand by the intervening hairpin adapter. In certain embodiments, the full length complementary daughter strand copy of the target fragment will include a single 3′ A overhang, as disclosed herein.

In an alternative embodiment, step 2 may be omitted from the method and adapter-ligated double stranded target fragment product 230 may be directly contacted with a strand-displacing DNA polymerase under nucleic acid synthesis conditions to initiate complementary copy strand synthesis off free 3′ ends 211a and 211b. In this instance, DNA synthesis proceeds as discussed with reference to FIG. 1B, step 2, until two separate adapter-ligated double stranded fragments are produced, each including one parental strand derived from the original double stranded target fragment and one complementary copy daughter strand covalently joined to the parental strand via the intervening hairpin adapter.

In certain embodiments, as discussed herein, extension reactions (i.e., the nucleic acid synthesis conditions) may include modified nucleotides that weaken the duplex strength of the extension products, i.e., the strength of the hydrogen bonds between the parent template strand and the newly synthesized daughter strand. Exemplary, non-limiting modified nucleotides are N4-Me dCTP and 7-deaza dGTP.

As shown in step 4, Y adapters 255 are then contacted with the adapter-ligated double stranded fragment 250 under nucleic acid ligation conditions and ligated to the double stranded end to produce a duplex template construct, as discussed with reference to FIG. 1D. Ligation of the Y adapters is facilitated by a free 3′ T overhang in the Y adapters that is capable of base-pairing with the single A overhang in the daughter stand of the extension products. In each duplexed template, the daughter strand is covalently coupled (i.e., covalently bound) to the parent strand by ligation to the intervening hairpin adapter. The duplexed template constructs may then be utilized for Sequencing by Expansion, as discussed with reference to FIG. 1.

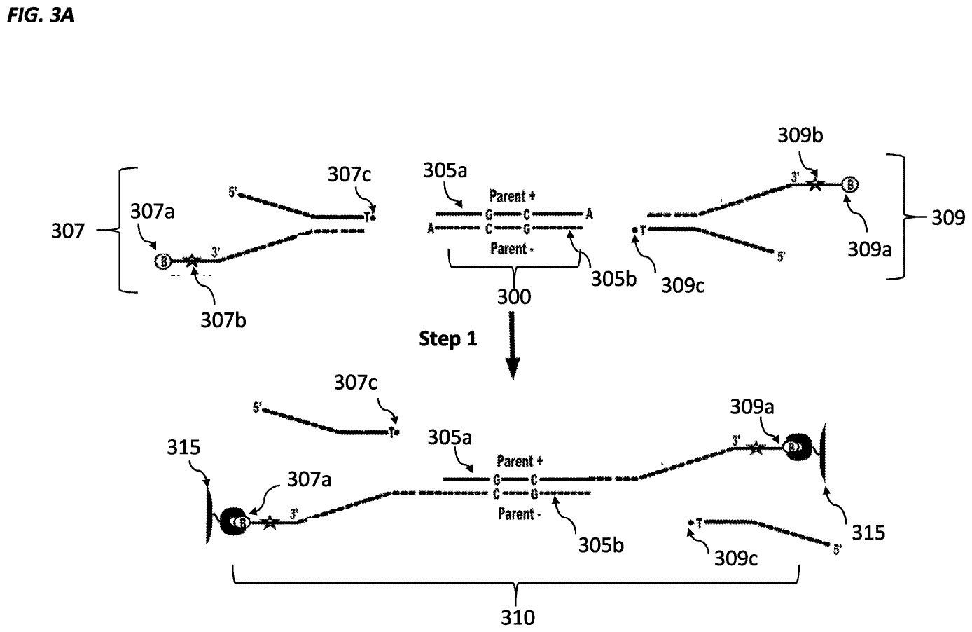

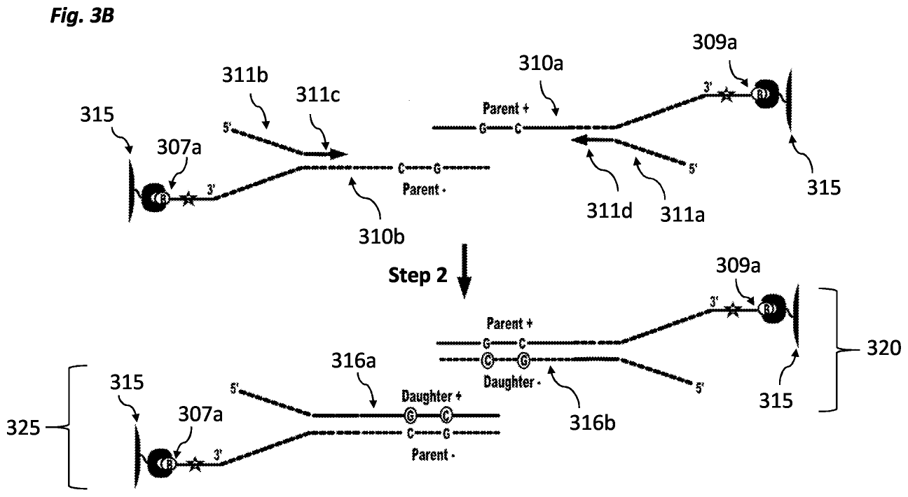

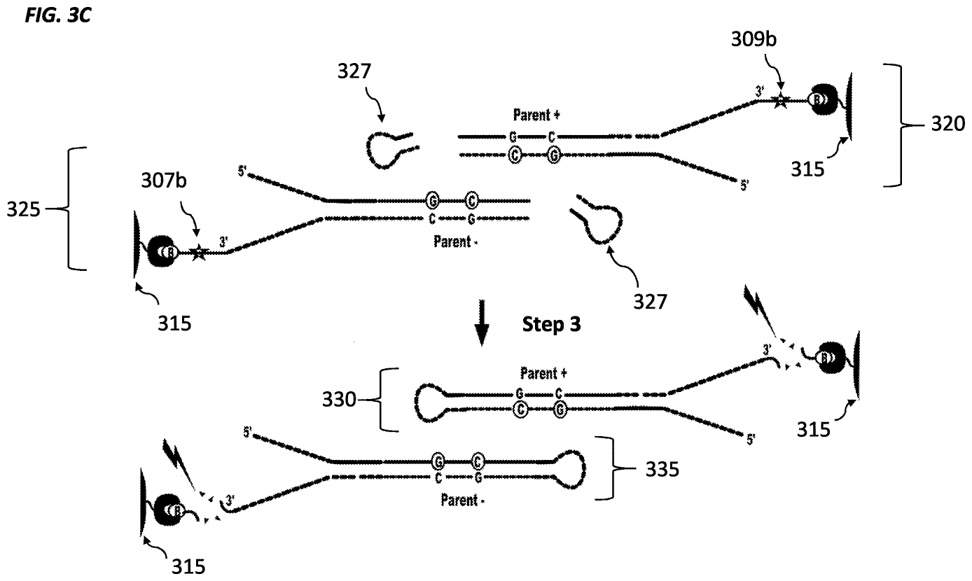

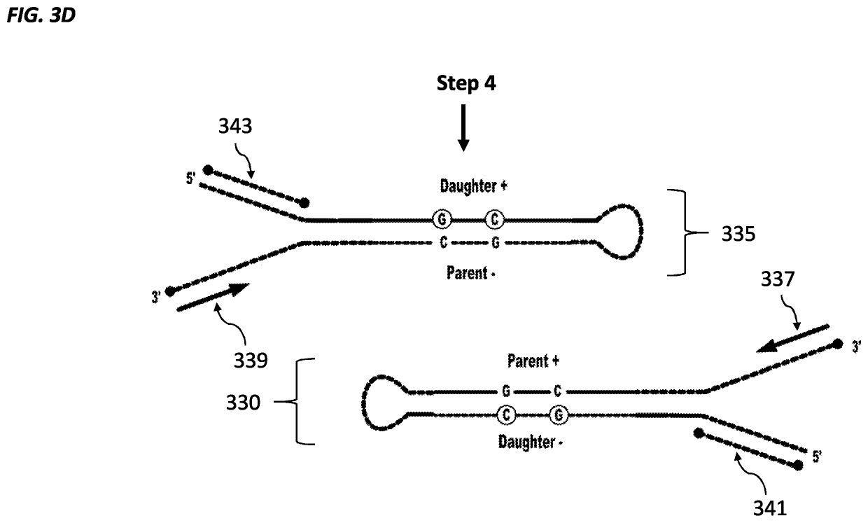

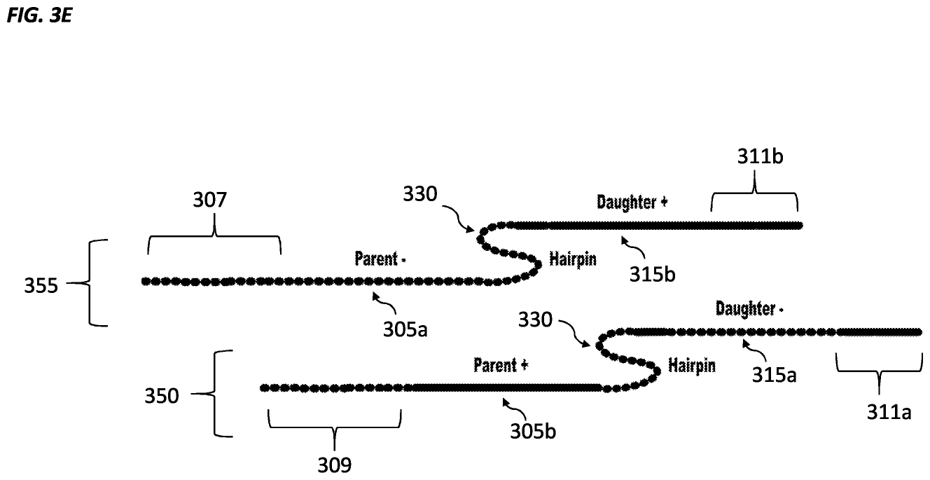

Another embodiment of a method of the present invention is set forth in FIG. 3. This embodiment provides the advantages of solid-state synthesis of duplex template constructs. As shown in FIG. 3A, a double stranded target fragment 300 is derived from genomic DNA or some other nucleic acid source. The double stranded target fragment 300 includes parent (+) strand 305A (i.e., a sense strand) and parent (−) strand 305B (i.e., an antisense strand). Prior to step 1, adapters 307 and 309 are ligated to the ends of the double stranded target fragment, as disclosed herein. In this embodiment, adapters 307 and 309 include features that enable solid-state synthesis. For example, the adapters may include biotin moieties 307a and 309a linked to the 3′ ends of the single stranded portion of the Y adapters and cleavable linkers 307b and 309b (here represented by star shapes) positioned between the biotin moieties and the 3′ ends of the adapters. In certain embodiments, the cleavable linkers may include a repeat of deoxyuridine that is cleavable by contact with a uracil DNA glycosylase (UDG) enzyme. The repeat of deoxyuridine, and other features such as one or more terminal biotin moieties, may be incorporated into an oligonucleotide strand of the Y adapter during synthetic oligonucleotide synthesis using art-recognized technology.

In certain embodiments, ligation of the adapters to the double stranded target fragment may be facilitated by including a 3′ single base overhang in one strand of the double stranded stem portion of the Y adapters (e.g., a single T overhang) and a complementary 3′ single base overhang in the opposite strand of the double stranded target fragment (e.g., a single A overhang). Base pairing of the T and A overhangs thus brings the adapter and target fragment into alignment for ligation. As shown in this embodiment, the adapters are Y adapters and each includes a region of double stranded DNA and a two regions of single stranded DNA. The adapters are joined to the double stranded target fragment such that one strand of the adapter is ligated to one strand of the target fragment, while the other strand of the adapter is not ligated to the target fragment. In this embodiment, the joining of the adapters to the double stranded target fragment is referred to as “partial ligation”. Partial ligation may be achieved, for example, by blocking the 3′ end of a single strand of the stem region of the Y adapter to prevent ligation to the 5′ ends of the parent sense strand of the target fragment. Blocking may be achieved in certain embodiments by phosphorylation of the 3′ ends of the Y adapter stem, as represented by solid circles 307c and 309c in Y adapter 307 and 309, respectively. Thus, the adapter-ligated double stranded target fragment product will include an internal single free 3′ end in each strand of the double stranded portion of the ligation product at the junction of the Y adapter and the target fragment.

In step 1, following partial ligation of the adapters to the double stranded target fragments, the strands of the Y adapters that include terminal blocked ends 307c and 309c are released from the target fragment. In some embodiments, this may be accomplished by applying denaturing conditions to the Y adapters. The opposite strands of the Y adapters that include terminal biotin moieties 307a and 309a, however, remain covalently associated with the double stranded target fragment. The adapter-ligated double stranded target fragment 310 may then be captured on a streptavidin-coated solid support 315. In other embodiments, the adapter-ligated double stranded target fragments are first captured on the solid support; subsequently, the single strands of the Y adapters are released via denaturation.

As shown in FIG. 3B, prior to step 2, the adapter-ligated double stranded target fragment may be treated with denaturing conditions to separate the two strands of the target fragment and produce single stranded adapter-ligated parent (+) strand 310a and single stranded adapter-ligated parent (−) strand 310b. Single stranded adapter-ligated parent (+) strand 310a and single stranded adapter-ligated parent (−) strand 310b may then be contacted with extendable primers 311a and 311b under nucleic acid hybridization conditions. In this embodiment, the extendable primers include a 3′ region (e.g., regions 311c and 311d) with a sequence that is designed to hybridize to the remaining single strand region derived from the Y adapter that remains ligated to the target fragment strand and a 5′ region that forms a single stranded arm. In this embodiment, the extendable primers resemble the original “top” strand of the Y adapter, except for the fact that they include an extendable free 3′ end.

In step 2, extendable primers, 311a and 311b, hybridized to single stranded adapter-ligated parent (+) strand 310a and single stranded adapter-ligated parent (−) strand 310b, are contacted with a DNA polymerase under nucleic acid extension conditions. DNA synthesis is initiated from the free 3′ OH group of the primers, using the single stranded parent fragments as templates. In certain embodiments, the extension reaction may include modified nucleotides that weaken (or strengthen, depending on the application) the strength of the hydrogen bonds between the parent template strand and the newly synthesized daughter strand. Exemplary, non-limiting modified nucleotides are N4-Me dCTP and 7-deaza dGTP. In embodiment illustrated in FIG. 3B, daughter strands 315a and 315b incorporate modified dGTP and dCTP analogs, as denoted by the circles around “G” and “C” in these strands.

The DNA polymerases proceed along the target fragment strands in opposite directions, producing double stranded products 320 and 325, each including one strand of the original double stranded target fragment (i.e., the parent strand) and one newly synthesized complementary copy strand (i.e., daughter strands 316a and 316b). Each of the double stranded products includes a fully ligated adapter at one end (i.e., there are no nicks or gaps between the target fragment and the adapter) and either a double stranded blunt end or single stranded overhang terminus at the opposite end. For example, the end opposite the adapter ligated end (i.e., lacking an adapter) is expected to be blunt if the polymerase traversed the entire target fragment strand, while it will have a single-stranded 5′ overhang in the parent strands if nucleic acid synthesis terminated before the polymerase reached the end of the target fragment parent strand. Further, if the polymerase employed has terminal transferase activity, there may be a 3′ overhang instead of a blunt end if the polymerase traverses the entire target fragment. No limitation in this regard is intended. In the embodiment depicted in FIG. 3, each adapter-ligated double stranded product is bound to the solid support via biotin moieties 307a and 309a joined to the 3′ arms of the Y adapters.

As shown in FIG. 3C, in step 3, hairpin adapters 327 are contacted with the double stranded extension products under DNA ligation conditions. The hairpin adapters are ligated to the double stranded ends of the extension products to produce duplex template constructs 330 and 335. Ligation of the hairpin adapters may be facilitated by single base overhangs, as discussed with reference to FIG. 1C. In each duplex template construct, the daughter strand is covalently joined to the parent strand via ligation of each strand to opposite ends of the intervening hairpin adapter. In certain embodiments, the duplex template constructs may be released from solid support 315 by breaking cleavable bond 307b and 309b. For example, UV light may be applied to cleave a photosensitive bond, as discussed further herein and depicted by the lightning bolt symbols illustrated in FIG. 3C.

As shown in FIG. 3D, in step 4, duplex template constructs 330 and 335 are prepared for Xpandomer synthesis. Advantageously, in certain embodiments, the duplex template constructs can be directly copied into Xpandomer products without prior PCR amplification. This reduces the likelihood of sequencing errors due to nucleotide misincorporations during amplification, particularly in regions of homopolymer sequence. Here, primers 337 and 339 are contacted with the duplex template constructs under nucleic acid hybridization conditions and hybridized to the 3′ ends of the single stranded arms of the Y adapters to provide initiation sites for Xpandomer synthesis. Blocker oligonucleotides 341 and 343 are likewise hybridized to the 5′ ends of the single stranded arms of the Y adapters to terminate Xpandomer synthesis.

Xpandomer synthesis reactions are carried out in which Xpandomer are synthesized from primers 337 and 339 in the 5′ to 3′ direction as the parent and daughter strands of the template are “unzipped”. Unzipping is facilitated by weak hydrogen bonding between strands due to modified G and C nucleotides included in the daughter strands. Full length Xpandomer copies 350 and 355 of duplexed templates 330 and 335, respectively are illustrated in FIG. 3E. From the 5′ to the 3′ direction, each Xpandomer will include a copy of the following sequences of the duplexed template: a copy of the sequence of the 3′ arm of the Y adapters (309 and 307, respectively); a copy of the sequence of the parent strand of the template (copy 305b of the parent sense strand and copy 305a of the parent antisense strand, respectively); a copy of the sequence of hairpin adapter (330); a copy of the sequence of the daughter strand copy of the template (copy 315a of the daughter antisense strand and copy 315b of the daughter sense strand); and a copy of the sequence of the extendable primers upstream of the blocker hybridization sequence (extendable primers 311a and 311b, respectively). As Xpandomers 350 and 355 are individually passed through a nanopore to obtain sequence information, the resulting sequencing reads will include information from both strands of the duplexed template in a single read, separated by the known sequence of the hairpin adapter that advantageously provides a “way point” to aid in bioinformatic sequence analysis. This redundancy in sequence information offers significant advantages in the ability to assess the quality of the read data. Moreover, optional inclusion of UMI barcodes in the Y adapters provides another level of quality assessment through the ability to bioinformatically link and compare the sequences of Xpandomer 350 and 355 that each include the sequence of a strand of the original nucleic acid target fragment.

The duplexed template constructs for Sequencing by Expansion, discussed with reference to FIG. 1-FIG. 3, each include a parental strand derived from the original double stranded DNA target fragment and a newly synthesized daughter complementary copy strand in which the two strands are covalently coupled by an intervening hairpin adapter. In certain alternative embodiments, the methods of the present invention include synthesis of a duplex template construct in which both strands of the original double stranded DNA target fragment are covalently coupled by an intervening hairpin adapter. Such duplex template constructs are referred to herein as “parent-parent” templates.

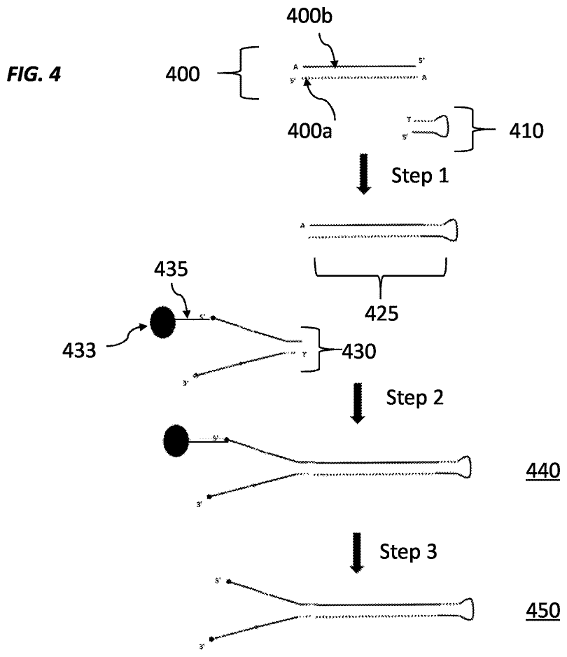

One method of generating a parent-parent template construct according to the present invention is depicted in FIG. 4. In this embodiment, library fragment (i.e., a double stranded DNA target fragment) 400 includes parental (sense) strand 400a and parental (antisense strand) 400b. The library fragment is end-repaired and A-tailed to generate single 3′ A overhangs in each strand, as discussed herein. In step 1, target fragment 400 is contacted with hairpin adapter 410, which includes a single 3′ T overhang to facilitate alignment with the target fragment, and a DNA ligase enzyme under DNA ligation conditions. The desired product of the ligation reaction is asymmetrically adapted duplex template construct 425 that includes a single hairpin adapter ligated to one end of the double stranded target fragment. In certain embodiments, the ratio of the hairpin adapter to the library fragment may be optimized to preferentially generate asymmetrically adapted duplex template construct 425 and to minimize generation of constructs with hairpin adapter ligated at both ends. In some embodiments, the molar ratio of hairpin adapter to library fragment insert may be from around 3:1 (“3×”) to up to around 10:1 (“10×”).

Prior to step 2, Y adapter 430 is immobilized on solid support 433 via linker 435 that is covalently bound at one end to the single stranded 5′ arm of the Y adapter and at the other end to the solid support. In this embodiment, linker 435 includes a poly dU sequence that is capable of being cleaved upon treatment with, e.g., the commercially available USER enzyme. Examples of suitable solid supports for the practice of the present invention are disclosed further herein. In some embodiments, a suitable solid support may be streptavidin-coated magnetic beads, e.g., “Dynabeads MyOne Streptavidin C1”, available from ThermoFisher Scientific, which are superparamagnetic beads of 1.0 m in diameter with a streptavidin monolayer covalently coupled to the hydrophilic bead surface.

In step 2, the ligation products of step 1, including asymmetrically adapter duplex template construct 425 are contacted with the immobilized Y adapter and a DNA ligase enzyme under DNA ligation conditions. Side products of the ligation reaction of step 2, including symmetrically adapted duplex templates (i.e., constructs with hairpin adapters ligated to both ends of the double stranded target fragment) are not capable of being ligated to the Y adapter, and thus will not be associated with solid support 433. In contrast, asymmetrically adapted duplex template construct 425 is capable of being ligated to Y adapter 430 to for Y adapter-ligated duplex template construct 440, bound to solid support 433 via linker 435.

In step 3, Y adapter-ligated duplex template construct 440 is treated with, e.g., a USER enzyme, to cleave the polyU sequence in linker 435 and release the Y adapter-ligated duplex template construct from the solid support. In other embodiments, linker 435 may include other suitable selectively-cleavable moieties known in the art, such as a photocleavable moiety. Released duplex template construct 450 may be further purified from the sample by art-recognized methods and kits, e.g., use of the KAPA Pure SPRI kit, as described herein.

In certain embodiments, synthesis of the parent-parent duplex template construct may be carried out entirely in-solution. For example, synthesis of the duplex template construct may include a first ligation reaction in which a hairpin adapter is ligated to a first end of a library fragment in-solution and a second ligation in which a Y adapter is ligated to a second end of the library fragment in-solution. After the first ligation reaction, products may be denatured, such that only library fragments with strands that are covalently joined (i.e., paired) by ligation to an intervening hairpin adapter will remain physically associated for the subsequent second ligation. Likewise, for the second ligation reaction, the only constructs capable of ligation to the Y adapter will be those in which the library fragment has a single free double stranded end. In certain embodiments, the ratio of the hairpin adapter to the target fragment in the first reaction may be optimized to preferentially generate ligation products including a single hairpin adapter (i.e., target fragments with a hairpin adapter ligated to one end only, with the other end remaining free of adapter). For example, the molar ratio of hairpin adapter to target fragment insert may be from around 2:1 (2×) to around 15:1 (15×), from around 3:1 (3×) to around 10:1 (10×), or from around 4:1 (4×) to around 8:1 (8×). In certain embodiments, an optional step of exonuclease-mediated digestion may be included after the second ligation step to remove undesired single stranded side products. Additional purification steps may be included in the workflow, e.g., one or more SPRI bead purification steps.

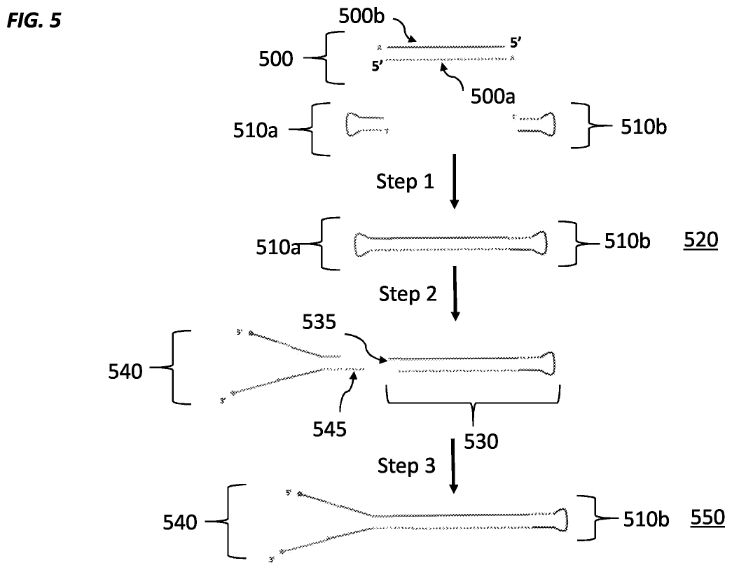

An alternative method of generating a parent-parent duplex template construct is depicted in FIG. 5. In this embodiment, library fragment (i.e., the double stranded DNA target fragment) 500 includes parental sense strand 500a and parental antisense strand 500b. The library fragment is end repaired and A-tailed to generate single 3′ A overhangs in each strand, as discussed herein. In step 1, the target fragment 500 is contacted with hairpin adapters 510a and 510b, which include a single T overhang to facilitate alignment with the target fragment, and a DNA ligase enzyme under DNA ligation conditions. In this embodiment one of the two hairpin adapters, e.g., adapter 510a, is designed to include a single stranded cleavage site in the sequence of each strand of the double stranded stem portion of the hairpin adapter. The sites are staggered in position such that cleavage of both sites leaves a single stranded overhang in one strand of the double stranded portion of the hairpin adapter. In certain embodiments, the cleavage sites may be recognition sites for a nicking endonuclease. Ligation product 520 is a dual hairpin adapter-ligated double stranded target fragment with cleavable hairpin adapter 510a joined to one end and non-cleavable hairpin adapter 510b joined to the opposite end of the target fragment.

In step 2, the dual hairpin adapter-ligated double stranded target fragment 520 is treated with conditions that generate single stranded breaks at the opposing cleavage sites in each strand of the double stranded stem region of the cleavable hairpin adapter 510a. In certain embodiments the conditions may include treatment with a nicking endonuclease. Then, the adapter-ligated double stranded target fragment is treated with denaturing conditions, e.g., by exposure to a strong base, heat or a combination thereof. The resulting product, asymmetric adapter-ligated double stranded target fragment 530 includes a single stranded overhang 535 that is derived from the sequence of cleavable hairpin adapter 510a.

Asymmetric adapter-ligated double stranded target fragment 530 is then contacted with Y adapter 540, which includes a single stranded overhang 545 in the double stranded portion of the adapter that is designed to be complementary to single stranded overhang 535 in the asymmetric adapter-ligated double stranded target fragment 530 and a DNA ligase under DNA ligation conditions. Ligation of Y adapter 540 to asymmetric adapter-ligated double stranded target fragment 530 generates duplexed template construct 550 with Y adapter 540 ligated to one end of the double stranded target fragment and hairpin adapter 510b ligated to the other end of the double stranded target fragment. Thus, duplex template construct 550 includes both strands of the original parental template covalently joined by intermediary non-cleavable hairpin adapter 510b.

The present invention provides several additional methods for generating parent-parent duplex template constructs. One such embodiment is referred to herein as “driven double ligation”. As used herein, the term “driven” means that certain reaction conditions, such as the molar ratio of the adapter to the target fragment insert (i.e., the double stranded nucleic acid fragment) and/or the temperature of the ligation reaction, may be optimized to drive the ligation reaction to completion. In certain embodiments, the molar ratio of adapter to target fragment may be from around 5:1 (5×) to around 10:1 (10×). In other embodiments, the molar ratio of adapter to target fragment may be up to around 100:1 (100×). In other embodiments, the temperature of the ligation reaction may be around 16 degrees C or more or up to around 20 degrees C.

According to the method, a first step may include minimal fragmentation of a sample of nucleic acids to produce double stranded DNA target fragments, e.g., minimal fragmentation of a sample of genomic DNA. In certain embodiments, minimal fragmentation may be achieved using any of the enzymatic or physical fragmentation techniques discussed herein, but with reduced enzyme concentration or timing of the fragmentation reaction. In some embodiments, minimal fragmentation may result in DNA target fragments with an average length of around 1000 nucleotides.

The method may include the second step of a first ligation reaction, referred to as the “driven double ligation reaction”. Here, a sample of hairpin adapters is contacted with a sample of DNA target fragments under driven DNA ligation conditions. As mentioned, the molar excess of hairpin adapter to DNA target fragments has the effect of driving the ligation reaction to completion, such that the DNA target fragments will be ligated at both ends to a hairpin adapter. Advantageously, the inventors have discovered that such driven conditions overcome the negative impact that certain sequences at the ends of the adapter and/or DNA target fragments have on the efficiency of the ligation reaction. For example, under certain conditions, the GC content of the sequence at the ends of the hairpin adapter can bias the ligation reaction such that the final quality of the sequencing data (e.g., library coverage) is negatively impacted. Therefore, the product of the driven double ligation is an intermediate duplex construct (i.e., a first ligation product) that includes a first hairpin adapter ligated to a first end and a second hairpin adapter ligated to a second end, i.e., a symmetrically adapter-ligated DNA target fragment. This reaction optimizes uniform representation across the library of DNA target fragment inserts and as well as the consistency and yield of intermediate duplex constructs.

The method may include the third step of fragmenting the sample of intermediate duplex template constructs. Accordingly, the sample of intermediate duplex constructs is treated with a fragmentation agent under fragmentation conditions to produce asymmetrically adapter-ligated duplex constructs in which the DNA target fragment is ligated to a hairpin adapter at just one end (with the newly created end at the fragmentation site being free of ligated adapter). In other words, the fragmentation products include a first end ligated to a first hairpin adapter and a second end lacking a second hairpin adapter. In certain embodiments, the fragmentation agent may be provided by a commercial kit, such as the KAPA EvoPlus V2 kit, available from Roche Sequencing Solutions, Inc. In certain embodiments, the fragmentation conditions are optimized to produce asymmetrically adapter-ligated duplex constructs with an average length of from around 100 bp to around 300 bp. The precise range of targeted fragment length will depend on the particular sequencing application. Following the fragmentation step, the free ends of the DNA target fragments may be end-repaired and A-tailed, as discussed herein. In certain embodiments, the sample of fragmented constructs may be subjected to size-selective purification, as discussed herein.

The method may include the fourth step of ligating a Y adapter to the free end of the asymmetrically adapter-ligated duplex template constructs. According to this step, the sample of asymmetrically adapter-ligated duplex constructs is contacted with a sample of Y adapters under DNA ligation conditions to produce a sample of duplex template constructs in which the DNA target fragment is ligated at a first end to the hairpin adapter and at a second end to the Y adapter. The ligation of Y adapters to one end of DNA target fragments is discussed further herein.

In other embodiments, the duplex template constructs of the present invention may be generated in a “one-pot” ligation reaction, i.e., in a single reaction vessel. In one embodiment, the ligation reaction may include a sample of DNA target fragments, a sample of Y adapters, a sample of hairpin adapters (all produced as discussed herein), a DNA ligase enzyme and a suitable buffer, salt, and/or other additives. The molar ratio of the Y adapters to the DNA target fragments and hairpin adapters may be optimized to drive the reaction to completion. The products of the one-pot ligation reaction will predominantly include constructs in which the DNA target fragments are ligated to a Y adapter on a first end and a Y adapter (single template) or a hairpin adapter (duplex template) on a second end. Both the single template and double template products function as suitable templates for Xpandomer synthesis. Xpandomers produced from the single template construct will generate sequence information from a single read of the DNA target fragment, while Xpandomers produced from the duplex template construct will generate sequence information from two reads of the DNA target fragment. Library preparation workflows implementing the one-pot ligation reaction provide several advantages to the overall sequencing workflow, including reducing the time to result.

In certain embodiments, the Sequencing by Expansion methodology may be streamlined in additional ways to reduce time-to-result. Exemplary adaptations include optimization and/or omission of certain steps of the overall workflow. For example, the library preparation workflows described herein may be streamlined into a workflow referred to as “SBX Fast” for applications requiring a rapid turnaround time, such as genomic analysis of high-risk newborn infants in a Neonatal Intensive Care Unit (NICU) clinical setting. In other embodiments, SBX Fast may be used for any clinical application requiring expedited sequence analysis.

In certain embodiments, a sample of blood of around 50 μL to 100 μL or more may be collected from a patient. The sample should yield from around 2 μg to around 4 μg of genomic DNA. In some embodiments the sample of genomic DNA will yield at least around 1.0 to 1.5 pmol of genomic DNA library for subsequent Xpandomer synthesis and nanopore sequence determination. In certain embodiments, samples of genomic DNA may also be obtained from one or more relatives (e.g., parents) of the patient or from unrelated individuals for the purpose of generating non-patient genomic DNA libraries in parallel to optimize downstream Xpandomer synthesis steps. Each individual library is distinguished by a unique SID identifier provided by the hairpin adapter of the library constructs. For example, a “proband” library (patient only) may be pooled with an unrelated, premade library that serves as “buffer” DNA during Xpandomer synthesis and sequencing. Alternatively, a “duo” library (one parent) or “trio” library (both parents) may be prepared in which library constructs derived from patient gDNA are pooled with library constructs derived from one or both of parental gDNA. The duo and trio libraries may assist in sequence analysis, e.g., to expedite identification of disease causing variants during tertiary analysis. Advantageously, the SBX Fast workflow does not require amplification of the DNA library. Not only does this reduce the overall time of library preparation, it also reduces the likelihood of propagating PCR-induced artificial mutations into the library fragments.

In certain embodiments, one or more time-consuming steps may be omitted from the library preparation workflow without negatively impacting library quality. For example, the wash steps that typically follow size-selective purification (e.g., the SPRI clean-up steps) may be omitted from the workflow. In addition, the time allocated for various steps or reactions may be reduced. For example, the incubation time of the first ligation reaction may be reduced; in certain exemplary embodiments, the time may be reduced to around 5, around 10, around 15, around 20 minutes, around 25, or around 30 minutes when the sample is incubated at around 15 degrees C to around 25 degrees C. Likewise, the incubation time of the second ligation reaction may be reduced to around 5, around 10, around 15, around 20 minutes, around 25, or around 30 minutes when the sample is incubated at around 15 degrees C to around 25 degrees C. In other embodiments, the first and second ligations may be pooled into a “one-pot” ligation reaction that is carried out in the same reaction vessel, e.g., the same tube.

In some embodiments, patient-derived and non-patient-derived libraries may be pooled for preparation of a sample of Xpandomer molecules for nanopore sequence determination. For example, libraries may be pooled into a single Xpandomer synthesis reaction and/or a single Xpandomer processing reaction in order to drive the relevant reaction(s) to completion and/or maximize recovery of mature Xpandomer molecules for passage through a nanopore sensor.

In certain embodiments, the time-to-result of the SBX Fast sequencing workflow may be from six to seven hours or less.

YSU Adapters and Adapter Combination Sets thereof. In one aspect, the present invention provides unique adapter configurations that can provide flexible adapter sets for duplex Sequencing by Expansion. For example, an adapter set may include a Y adapter species and a hairpin adapter species. These unique adapters may be used in multiple combinations to support a wide range of sequencing applications. In certain embodiments, the adapter set may include 1) a Y adapter that includes one or more primer hybridization sequences and one or more nickase recognition sequences; 2) a “YSU” adapter that includes one or more primer hybridization sequences, one or more nickase recognition sequences, a UMI sequence, and a SID sequence; and 3) a hairpin adapter that includes a SID sequence (“UPS”). Advantageously, these adapter designs are compatible with linear amplification, PCR, and PCR-free library preparation and target enrichment workflows.

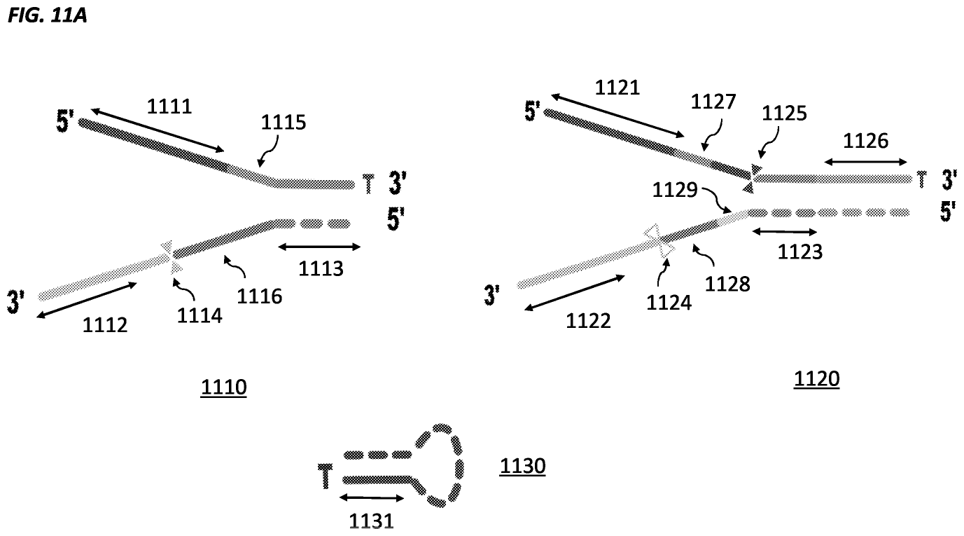

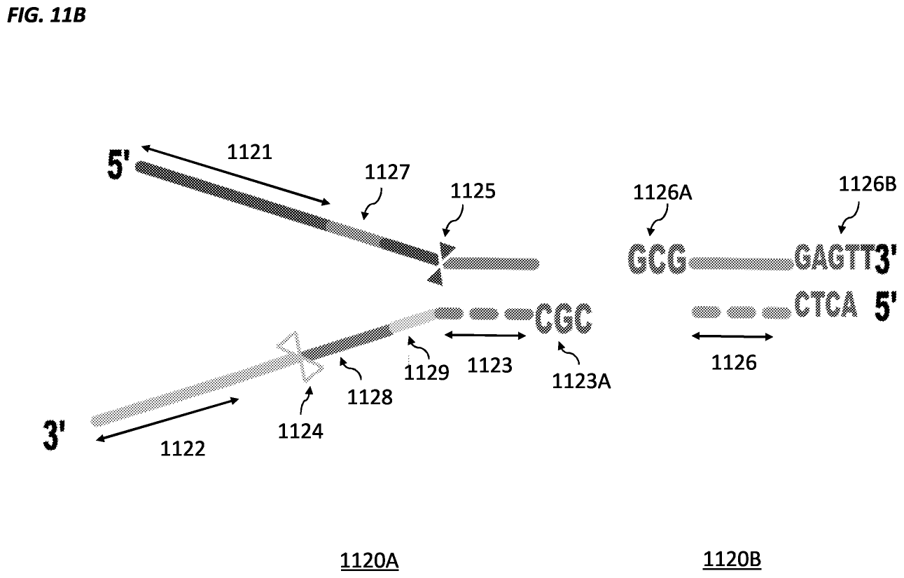

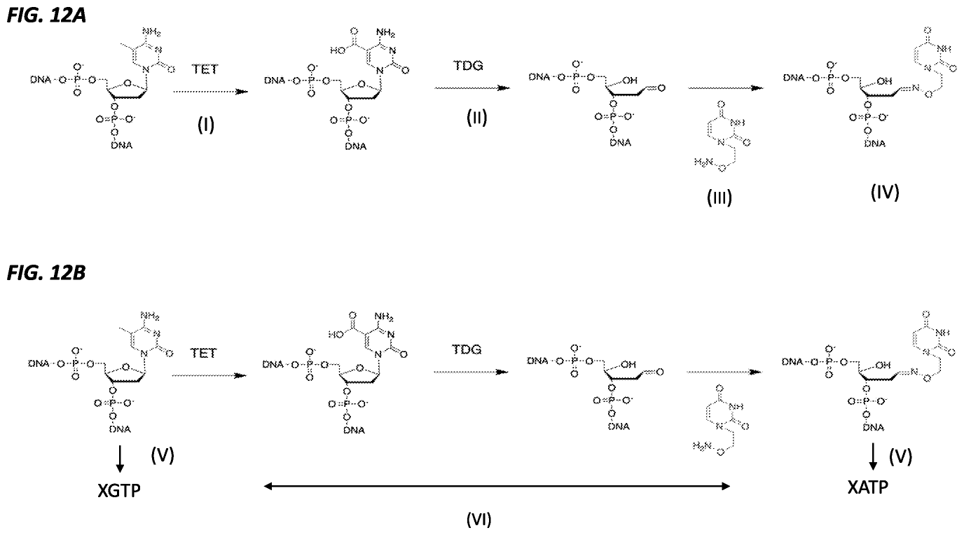

Non-limiting examples of these three adapter configurations are illustrated in FIG. 11A. In certain embodiments, Y adapter 1110 may include one or more of the following features: extension oligonucleotide hybridization sequence 1111, here positioned in the 5′ single stranded arm of the Y adapter; amplification primer hybridization sequence 1112, here positioned in the 3′ single stranded arm of the Y adapter; SID sequence 1113, here positioned in the double stranded stem region of the Y adapter; first nickase site 1114, here positioned in the 3′ single stranded arm of the Y adapter; Xpandomer synthesis runway sequence 1115, here positioned in the 5′ single stranded arm of the Y adapter. The Xpandomer runway sequence is the first sequence that is copied into the Xpandomer and is designed to facilitate initiation of Xpandomer synthesis by the polymerase. In certain embodiments, the sequence includes bases that are easily recognized and copied by the polymerase. In one embodiment, the Xpandomer runway sequence may be CAACAA or a variant thereof. The YSU adapter may further include blocker/cap hybridization sequence 1116, here positioned in the 3′ single stranded arm of the Y adapter.

In certain embodiments, YSU adapter 1120 may include one or more of the following features: extension oligonucleotide hybridization sequence 1121, here positioned in the 5′ single stranded arm of the Y adapter; amplification primer hybridization sequence 1122, here positioned in the 3′ single stranded arm of the Y adapter; SID sequence 1123, here positioned in the double stranded stem region of the Y adapter; first nickase site 1124, here positioned in the 3′ single stranded arm of the Y adapter; second nickase site 1125, here positioned in the 5′ single stranded arm of the Y adapter; UMI sequence 1126, here positioned in the double stranded stem region of the Y adapter; Xpandomer synthesis runway sequence 1127, here positioned in the 5′ single stranded arm of the Y adapter; blocker/cap hybridization sequence 1128, here positioned in the 3′ single stranded arm of the Y adapter; and capping runway sequence 1129, here positioned in the 3′ single stranded arm of the Y adapter. The capping runway sequence is the last sequence that is copied by the polymerase into the Xpandomer and is designed to facilitate joining of the blocker oligonucleotide to the Xpandomer by the polymerase. In certain embodiments, the capping runway sequence may be AAA or the like.

In certain embodiments, HPS adapter 1130 may include SID sequence 1131, here positioned in the double stranded stem region of the hairpin adapter.

In one non-limiting embodiment, the adapter features may have the following approximate lengths: extension oligonucleotide binding site: around 23 nucleotides; Xpandomer synthesis runway: around 6 nucleotides; amplification priming site: around 18 nucleotides; first nickase site: around 8 nucleotides; second nickase site: around 9 nucleotides; blocker/cap hybridization regions: around 24 nucleotides (for the Y adapter) or around 10 nucleotides (for the YSU adapter); UMI sequence: around 6 nucleotides; capping runway: around 3 nucleotides; SID sequence: around 16 nucleotides (for the Y adapter) or around 12 nucleotides (for the YSU and hairpin adapters).

FIG. 11B summarizes a combinatorial method for introducing UMI sequences into YSU adapters. Here, YSU adapter arm portion 1120A includes all features described with reference to FIG. 11A, except for the UMI sequence, which is provided in YSU adapter stem portion 1120B. In one embodiment, a pool of unique YSU adapter arm portions may be generated that each include a unique SID sequence; likewise, a pool of unique YSU adapter stem portions may be generated that each include a unique UMI sequence. The 5′ end of the 3′ arm of the YSU adapter arm region may be designed to include overhang 1123A of around 3 bases and the UMI stem portion may be designed to include overhang 1126A of around 3 bases with a sequence that is complementary to overhang 1123A. The UMI stem portion may be designed to also include sequence 1126B to facilitate downstream steps in the library prep workflow.

In certain embodiments, a pool of YSU adapters may be produced by contacting each unique YSU adapter arm portion with the pool of unique YSU adapter stem portions and a DNA ligase enzyme under DNA ligation conditions to provide the pool of YSU adapters. Each adapter will contain a unique stem sequence that can be use bioinformatically as an SID, UMI, or neither, depending on the application. In one embodiment, the pool of YSU adapters will include the same SID sequence, provided by the YSU arm portion and a unique UMI sequence, provided by the YSU stem portion.

Advantageously, this modular approach to generating YSU adapters utilizes smaller input oligonucleotides relative to conventional methods of generating Y adapters, thus reducing cost of goods and increasing the ultimate purity of the oligonucleotides. As such, the quality control of the input oligonucleotides does not need to be as stringent as that of conventional methods, as the DNA ligase enzyme creates the final full length Y adapter strands. Moreover, according to the present method, the SID sequences are added to the Y adapter during the ligation step, thus eliminating the need to include a separate PCR step to introduce oligonucleotides including SID sequences into the Y adapter, which is a limitation of conventional library preparation workflows.

It is to be emphasized that a pool of any number of unique YSU adapters may be generated according to the combinatorial methods of present invention; no limitation is intended in this description.

### Duplex Template Construct Opening

The duplex template constructs of the present invention may, in some instances, be “unzipped” or “opened” prior to initiating Xpandomer synthesis. For example, the hydrogen bonds between the sense and antisense strands of the double stranded target fragment may be broken to provide one or more regions of single stranded DNA template. This is referred to herein as “duplex opening”. Providing the polymerase with a single stranded template for Xpandomer synthesis advantageously reduces the requirement for strand displacement activity.

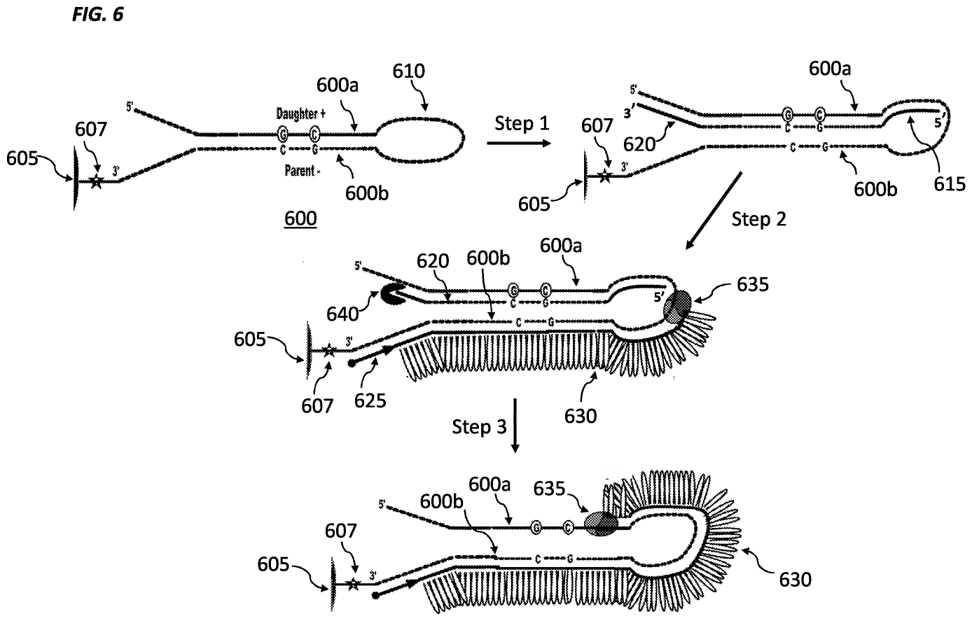

One example of duplex opening is depicted in FIG. 6. In this embodiment, duplex template construct 600 includes a double stranded target fragment with daughter (+) strand 600a (i.e., a sense strand) and parent (−) strand 600b (i.e., an antisense strand). In certain embodiments, the daughter strand may include modified nucleotides that weaken the strength of the hydrogen bonding with the complementary strand, as described herein (and denoted by the circles around the “G” and “C” in daughter strand 600a). The double stranded target fragment is joined at a first end to a hairpin adapter that covalently links daughter (+) strand 600a and parent (−) strand 600b. The single stranded region of the hairpin adapter includes a nucleotide sequence 610 that is complementary to, and provides a hybridization site for, an oligonucleotide primer, which may be referred to herein as an “invasive” primer. The double stranded target fragment is joined at a second end to a Y adapter, as described herein. In some embodiments, the Y adapter may be bound to a solid support 605 via a cleavable linker 607.

In step 1, an invasive oligonucleotide primer 615 is hybridized to the complementary nucleotide sequence 610 in the hairpin adapter and contacted with a strand displacing DNA polymerase under nucleic acid synthesis conditions. The DNA polymerase begins synthesizing a complementary copy of daughter (+) strand 600a from the 3′ end of the invasive oligonucleotide primer towards the terminal 5′ end of the Y adapter to produce second daughter (−) strand 620. During synthesis of the second daughter (−) strand, the original parent (−) strand 600b is displaced (e.g., unzipped) from the original daughter (+) strand 600a. In other words, original parent (−) strand 600b is no longer hybridized to daughter (+) strand 600a. This provides the parent (−) strand 600b as a single stranded, more accessible, template for Xpandomer synthesis.

In step 2, synthesis of an Xpandomer copy of the duplex template construct is initiated. First, extension oligonucleotide 625 is hybridized to a complementary sequence at the end of the 3′ single stranded arm of the Y adapter, which is bound to the solid support 605 in this embodiment. The extension oligonucleotide is contacted with DNA polymerase 635 that is capable of synthesizing an Xpandomer copy of the template (e.g., a variant of DPO4 polymerase, as disclosed herein) and Xpandomer synthesis conditions. The polymerase begins synthesis of Xpandomer 630 from the 3′ end of the extension oligonucleotide using parent (−) strand 600b as the template. Advantageously, as described, Xpandomer synthesis does not require displacement of a non-template strand from a double-stranded target fragment. In certain embodiments, step 2 also includes the step of contacting the second daughter (−) strand 620 with enzyme 640 possessing 3′->5′ exonuclease activity under exonuclease conditions. In certain embodiments, enzyme 640 may be, e.g., Exonuclease III or a suitable DNA polymerase or helicase that possesses 3′->5′ exonuclease activity on double stranded DNA. Enzyme 640 initiates digestion of second daughter (−) strand 620 from the terminal 3′ end until it reaches the opposite 5′ end of the strand, thereby exposing daughter strand 600a as a single strand, which provides a more readily accessible template for the DNA polymerase.

In step 3, enzyme 640 has completed exonuclease-mediated digestion of second daughter (−) strand 620, thereby opening up the template for polymerase 635 to continue synthesis of Xpandomer 630. The final Xpandomer product will thus include a copy of both strands of the original double stranded target fragment covalently linked by a copy of the intervening hairpin adapter. The Xpandomer product may be released from the solid support 605 by cleavage of the cleavable bond 607, as disclosed herein.

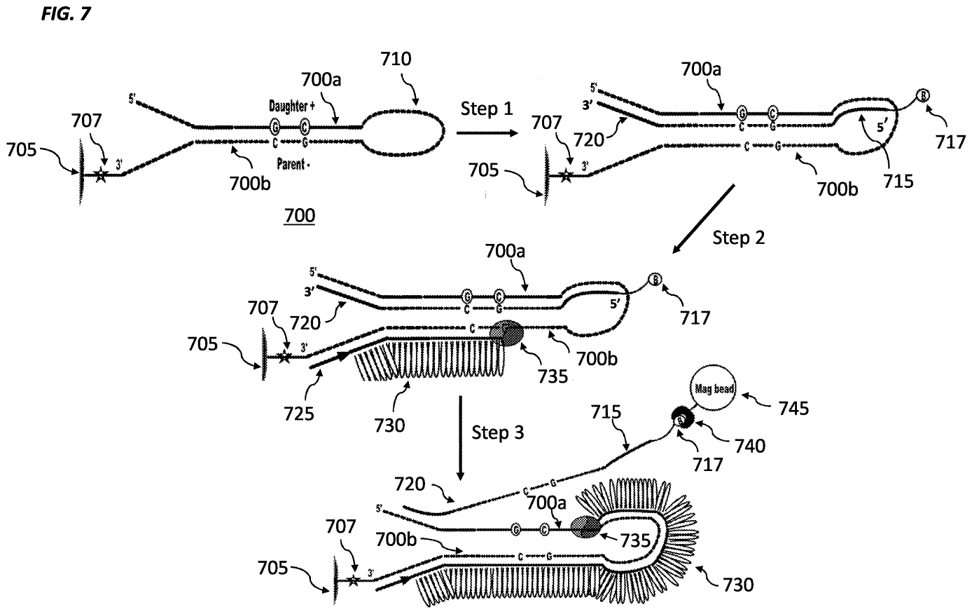

One alternative example of duplex opening is depicted in FIG. 7. In this embodiment, duplex template construct 700 includes a double stranded target fragment with daughter (+) strand 700a (i.e., a sense strand) and parent (−) strand 700b (i.e., an antisense strand). In certain embodiments, the daughter strand may include modified nucleotides that weaken the strength of the hydrogen bonding to the complementary strand, as described as herein (and denoted by the circles around the “G” and “C” in daughter strand 700a). The double stranded target fragment is joined at a first end to a hairpin adapter that covalently links daughter (+) strand 700a and parent (−) strand 700b. The single stranded region of the hairpin adapter includes a nucleotide sequence 710, which is complementary to, e.g., the nucleotide sequence of an invasive oligonucleotide primer. The double stranded target fragment is joined at a second end to a Y adapter, as described herein. In some embodiments, the Y adapter may be joined to a solid support 705 via a cleavable linker 707.

In step 1, an invasive oligonucleotide primer 715 is hybridized to the complementary nucleotide sequence 710 in the hairpin adapter. In this embodiment, the invasive oligonucleotide primer 715 include a terminal biotin moiety 717 operably linked to the 5′ end of the primer. In certain embodiments, the terminal biotin moiety 717 may be operably linked to the 5′ end of the primer via a flexible linker, e.g. a repeat of PEG6 spacers. The invasive oligonucleotide primer is contacted with a strand displacing DNA polymerase under nucleic acid synthesis conditions. The DNA polymerase begins synthesizing a complementary copy of daughter (+) strand 700a from the 3′ end of the invasive oligonucleotide primer until it reach the terminal 5′ end of the Y adapter to produce second daughter (−) strand 720. During synthesis of the second daughter (−) strand, the original parent (−) strand 700b is displaced (e.g., unzipped) from the original duplexed target fragment. In other words, original parent (−) strand 700b is no longer hybridized to daughter (+) strand 700a. As discussed, this advantageously provides the parent (−) strand 700b as a single stranded template for a polymerase.

In step 2 synthesis of an Xpandomer copy of the duplex template construct is initiated. First, extension oligonucleotide 725 is hybridized to a complementary sequence at the 3′ end of the Y adapter, which is bound to the solid support 705 in this embodiment. The extension oligonucleotide is contacted with DNA polymerase 735 that is capable of synthesizing an Xpandomer copy of the template (e.g., a variant of DPO4 polymerase, as disclosed herein) and Xpandomer synthesis conditions. The polymerase begins synthesis of Xpandomer 730 from the 3′ end of the extension oligonucleotide using parent (−) strand 700b as a template. Advantageously, as described, Xpandomer synthesis does not require displacement of a non-template strand from a double-stranded target fragment.

In step 3, the terminal biotin moiety 717 of the invasive oligonucleotide primer is contacted with streptavidin moiety 740 that is covalently coupled to magnetic bead 745. The tight association of the biotin and streptavidin moieties enables second daughter (−) strand 720 to be physically dissociated from the original daughter (+) strand 700a by applying an appropriate force to magnetic bead 745. Dissociation of second daughter (−) strand 720 from the original duplex provides daughter (+) strand 700a as a single stranded template, enabling polymerase 735 to continue synthesis of Xpandomer 730. As discussed with reference to FIG. 6, the final Xpandomer product contains a copy of both strands of the original double stranded target fragment covalently linked by a copy of the intervening hairpin adapter. The Xpandomer product may be released from the solid support 705 for nanopore sequencing by cleavage of the cleavable bond 707, as disclosed herein.

In certain embodiments, the duplex template construct opening methods discussed with reference to FIGS. 6 and 7 may be used for one or more target enrichment steps prior to Xpandomer synthesis. In general terms, target enrichment enables, e.g., targeted sequencing of just the coding regions or specific genes or segments of chromosomes that are relevant to a particular disease. With this approach, the rest of the whole genome can be disregarded, simplifying downstream bioinformatics analysis and making the sequencing workflow more efficient and reducing the overall cost.

Various target enrichment methods are known in the art, including hybridization capture-based target enrichment and primer extension-based target enrichment (see, e.g., the KAPA HyperCap work-flow and the KAPA HyperPETE work-flow, both commercially available from Roche Sequencing Solutions, Inc.). Dual primer extension based target enrichment (PETE) is described in greater detail in Applicant's U.S. Pat. No. 11,773,388, the contents of which are herein incorporated by reference in their entirety.

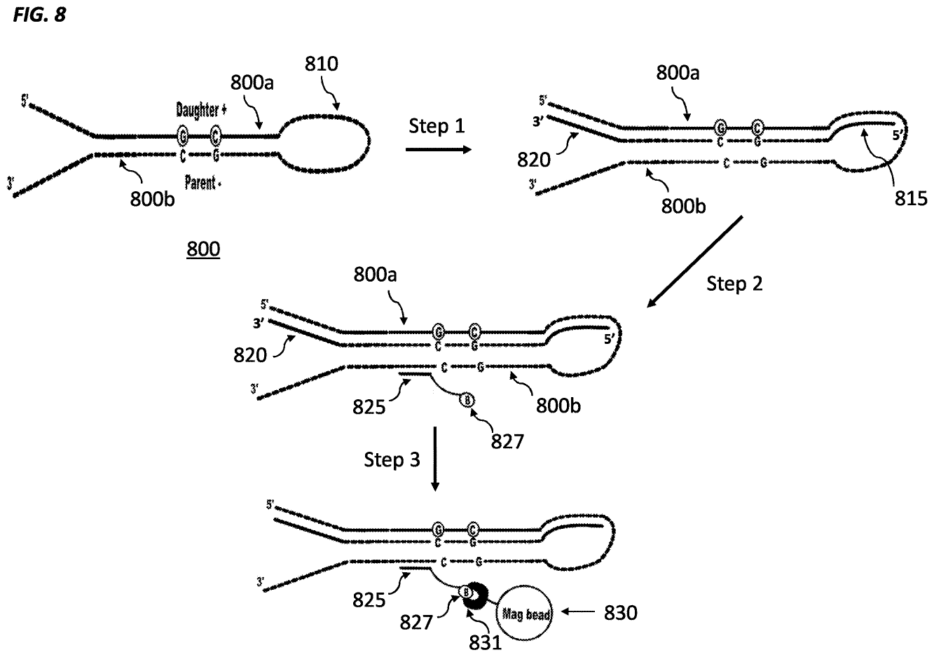

One embodiment of duplex opening for target enrichment is depicted in FIG. 8. In this embodiment, duplex template construct 800 includes a double stranded target fragment with daughter (+) strand 800a (i.e., a sense strand) and parent (−) strand 800b (i.e., an antisense strand). In certain embodiments, the daughter strand may include modified nucleotides that weaken the strength of the hydrogen bonding with the complementary strand, as described herein (and denoted by the circles around the “G” and “C” in daughter strand 800a). The double stranded target fragment is joined at a first end to a hairpin adapter that covalently links daughter (+) strand 800a and parent (−) strand 800b. The single stranded region of the hairpin adapter includes a nucleotide sequence 810 that is complementary to, and provides a hybridization site for, an oligonucleotide primer that may be referred to herein as an “invasive” primer. The double stranded target fragment is joined at a second end to a Y adapter, as described herein. In some embodiments, the Y adapter may be bound to a solid support via a cleavable linker.

In step 1, an invasive oligonucleotide primer 815 is hybridized to the complementary nucleotide sequence 810 in the hairpin adapter and contacted with a strand displacing DNA polymerase under nucleic acid synthesis conditions. The DNA polymerase begins synthesizing a complementary copy of daughter (+) strand 800a from the 3′ end of the invasive oligonucleotide primer towards the terminal 5′ end of the Y adapter to produce second daughter (−) strand 820. During synthesis of the second daughter (−) strand, the original parent (−) strand 800b is displaced (e.g., unzipped) from the original daughter (+) strand 800a. In other words, original parent (−) strand 800b is no longer hybridized to daughter (+) strand 800a. This provides the parent (−) strand 800b as a free single strand and a more accessible template for Xpandomer synthesis.

In step 2, oligonucleotide probe 825 is contacted with and a complementary sequence in parent (−) strand 800b under nucleic acid hybridization conditions. The oligonucleotide probe is designed to have a sequence complementary to a known sequence of interest, which may or may not be represented in the entire library of duplex template constructs. In this embodiment, duplex template construct 800 includes the sequences of interest, and is thus a target for enrichment from the larger pool of duplex template constructs.

In certain embodiments, the length of the oligonucleotide probe 825 is designed to enable target enrichment by hybridization capture. The parameters determining oligonucleotide design for target enrichment by hybridization capture are well known in the art and many probe kits and panel design tools are commercially available (e.g., from Roche Sequencing Solutions, Inc.). In certain embodiments, the length of the oligonucleotide 825 probe may be from around 50 bp to around 250 bp, from around 75 bp to around 200 bp, from around 100 bp to around 175 bp, or from around 120 bp to around 150 bp. In other embodiments, the sequence of the probe may include a GC content of around 40% to around 60%, from around 45% to around 55%, or around 50% or less.

In step 3, in certain embodiments, oligonucleotide probe 825 includes additional features that enable selective enrichment of target fragments of interest; for example, the probe may be operably linked to biotin moiety 827 that enables isolation of the hybridized duplex template construct by binding to streptavidin moiety 831 operably linked to magnetic bead 830, using conventional techniques. The entire work-flow for target enrichment by hybridization capture is described in Roche's instructions for use manual, KAPA HyperCap Workflow v3.2 (September, 2021), the contents of which are herein incorporated by reference in its entirety.

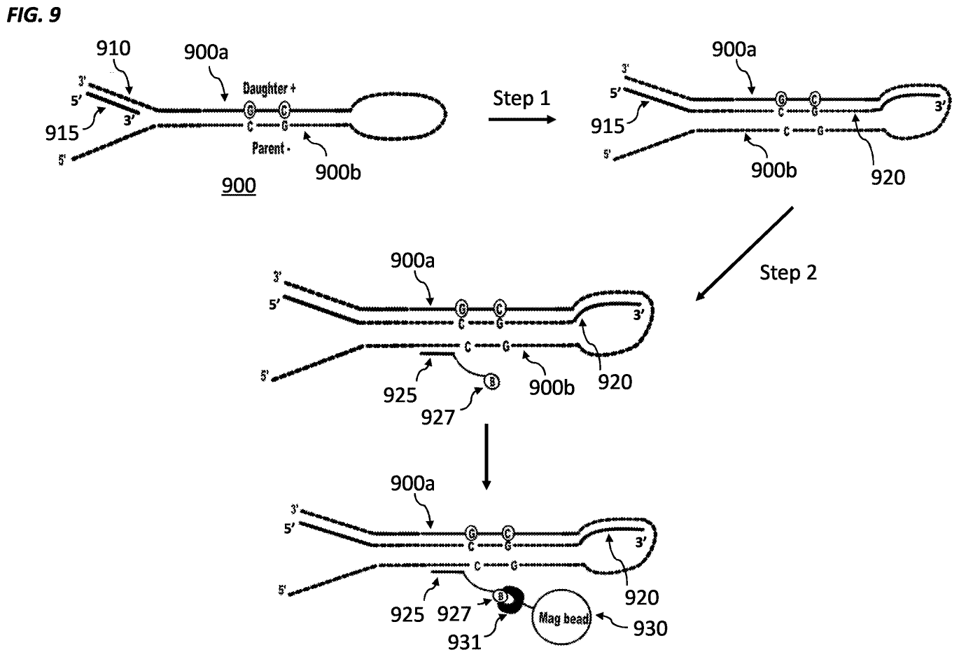

Another embodiment of duplex opening for target enrichment is depicted in FIG. 9. In this embodiment, the polarity of duplex template construct 900 is reversed relative to that of the duplex template construct discussed with reference to FIG. 8. Here, duplex template construct 900 includes a double stranded target fragment with daughter (+) strand 900a (i.e., a sense strand) and parent (−) strand 900b (i.e., an antisense strand). In certain embodiments, the daughter strand may include modified nucleotides that weaken the strength of the hydrogen bonding with the complementary strand, as described herein (and denoted by the circles around the “G” and “C” in daughter strand 900a). The double stranded target fragment is joined at a first end to a hairpin adapter that covalently links daughter (+) strand 900a and parent (−) strand 900b. The double stranded target fragment is joined at a second end to a Y adapter, as described herein. In this embodiment, the 3′ end of the single stranded portion of the Y adapter includes a sequence 910 that is complementary to the sequence of an extension oligonucleotide 915. Duplex template construct 900 is contacted with and hybridized to extension oligonucleotide 915. In some embodiments, the Y adapter may be bound to a solid support via a cleavable linker.

In step 1, extension oligonucleotide 915 is contacted with a strand displacing DNA polymerase under nucleic acid synthesis conditions. The DNA polymerase begins synthesizing a complementary copy of daughter (+) strand 900a from the 3′ end of the extension oligonucleotide towards the hairpin adapter to produce second daughter (−) strand 920. During synthesis of the second daughter (−) strand, the original parent antisense strand 900b is displaced (e.g., unzipped) from the original daughter (+) strand 900a. In other words, original parent (−) strand 900b is no longer hybridized to daughter (+) strand 900a. Synthesis of second daughter strand 920 is terminated once the polymerase has encountered a termination feature within in the hairpin adapter sequence. For example, certain termination sequences or other nucleic acid synthesis termination features can be engineered into the design of the hairpin adapter structure using art-recognized technologies. This provides the parent (−) strand 900b as a free single strand, which is a more accessible template for Xpandomer synthesis.

In step 2, oligonucleotide probe 925 is contacted with parent antisense strand 900b under nucleic acid hybridization conditions. As mentioned with reference to FIG. 8, the oligonucleotide probe is designed to have a sequence complementary to a known sequence of interest, which may or may not be represented in the entire library of duplex template constructs. In this embodiment, duplex template construct 900 includes the sequences of interest that is the target for enrichment from the larger pool of duplex template constructs.

As discussed with reference to FIG. 8, the features of the probe may be designed to enable target enrichment by hybridization capture. In other embodiments, the length of the oligonucleotide probe 925 may be designed to enable target enrichment by primer extension. The parameters determining oligonucleotide design for target enrichment by primer extension are also well known in the art and, likewise, many probe kits and panel design tools are commercially available (e.g., from Roche Sequencing Solutions, Inc.). In certain embodiments, the length of the oligonucleotide probe may be from around 15 bp to around 75 bp, from around 20 bp to around 70 bp, from around 20 bp to around 25 bp, or from around 50 bp to around 60 bp. In other embodiments, the sequence of the probe may include a GC content of around 40% to around 60%, from around 45% to around 55%, or around 50% or less.

With continued reference to FIG. 9, in certain embodiments, oligonucleotide probe 925 includes additional features that enable selective enrichment of duplex template construct 900; for example, the probe may be operably linked to biotin moiety 927 that enables isolation of the hybridized duplex template construct by binding to streptavidin moiety 931 operably linked to magnetic bead 930, as illustrated in step 3. For PETE based target enrichment, probe 925 is extended by a DNA polymerase prior to enrichment. The enriched duplexed template construct may then be released from the magnetic bead 930 following capture by a second primer extension reaction using a release primer that hybridizes to the parent (−) strand 900b at a position 5′ to the capture primer (i.e., the oligonucleotide probe 925). Extension of the release primer by a DNA polymerase releases duplex template construct 900 from magnetic bead 930 into solution. The entire work-flow for target enrichment by dual primer extension is described in Roche's instructions for use manual, KAPA HyperPETE Somatic Tissue DNA Workflow v1.0 (July, 2021), the contents of which are herein incorporated by reference in its entirety.

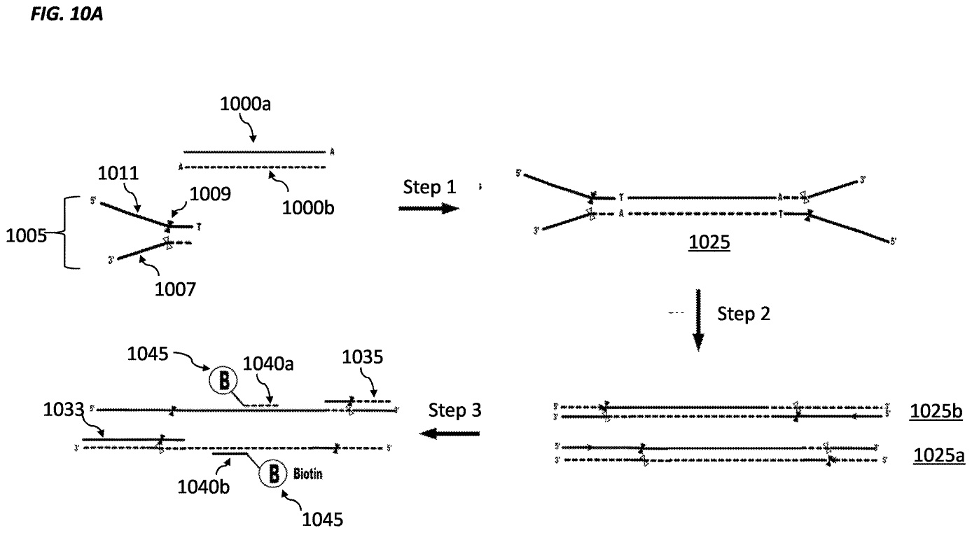

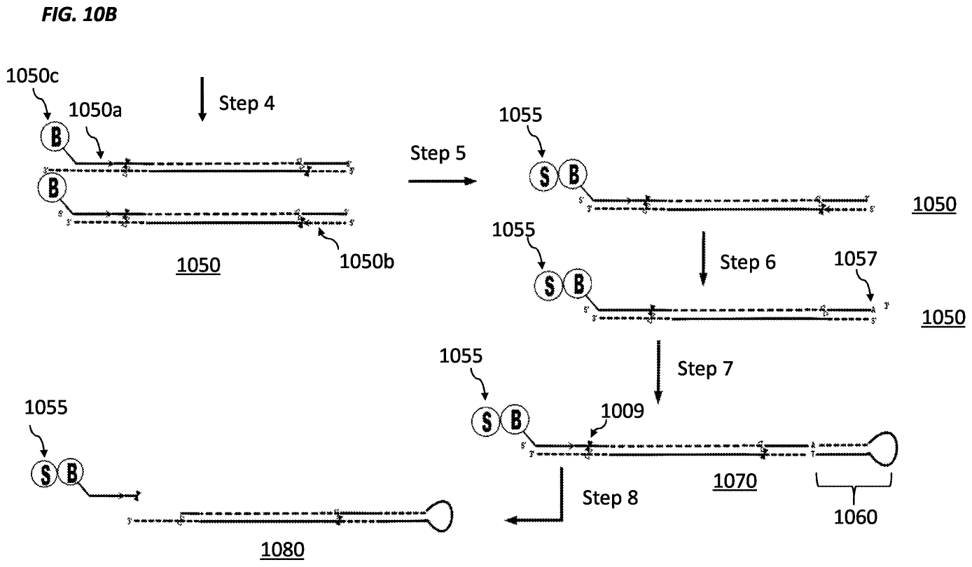

In certain embodiments, the methods of the present invention may include a workflow in which a target enrichment step is introduced into the duplex template construct library preparation workflow. One example of a method of duplex template construct preparation integrating a target enrichment step is illustrated in simplified form in FIGS. 10A and 10B. Here, a double stranded DNA target fragment (i.e., a library fragment) is provided that includes parent (+) strand (i.e., a sense strand) 1000a and parent (−) strand (i.e., an antisense strand) 1000b. The nucleic acid target fragment is end-repaired and A-tailed, as described herein. Also provided are Y adapters 1005, which, in certain embodiments, may include any suitable sequence for a particular library preparation or sequencing application, such as, in this embodiment, nickase recognition site 1009. The 3′ end of the double stranded region of the Y adapters may also include a single base T overhang to facilitate alignment and ligation to the library fragment. The Y adapters include 3′ single stranded arm region 1007 and 5′ single stranded arm region 1011. In step 1, the double stranded nucleic acid target fragment is contacted with the Y adapters with a DNA ligase under DNA ligation conditions. Each end of the double stranded DNA target fragment is ligated to a Y adapter to generate library construct 1025.

In step 2, library construct 1025 is subjected to a first round of PCR amplification. In certain embodiments, forward primers are provided with a sequence complementary to a sequence in the 3′ single stranded arm (1007) of the Y adapters and reverse primer are provided with a sequence that is identical to a sequence in the 5′ single stranded arm (1011) of the Y adapters. The products of the first PCR reaction include first PCR product 1025a, in which the original template included the sense strand 1000a of the library fragment and second PCR product 1025b, in which the original template included the antisense strand 1000b of the library fragment and

In step 3, the population of PCR products are subjected to a target enrichment step. In this embodiment, the population of PCR products includes the population of first PCR products. In other embodiments, the population of PCR products may include the population of second PCR product. Blocker oligonucleotides are provided with sequences that are complementary to sequences at the 3′ ends of each strand of the PCR products. Shown here, first blocker oligonucleotide 1035 is complementary to the 3′ end of the sense strand of PCR product 1025a, while second blocker oligonucleotide 1033 is complementary to the 3′ end of the antisense strand of the same PCR product. The population of PCR products is denatured and contacted with the blocker oligonucleotides under nucleic acid hybridization conditions that favor hybridization of the blocker oligonucleotides to the single stranded PCR products over re-hybridization of two strands of the PCR products. The blocker oligonucleotides are designed to hybridize to sequences derived from the original Y adapters and advantageously prevent non-specific carry-over of non-target sequences during target enrichment. Target-specific hybridization probes (e.g., capture probes) are then contacted with the pool of single stranded PCR products under suitable nucleic acid hybridization conditions. In some embodiments, target-specific hybridization probes are designed with sequences that are complementary to either the sense strand or the antisense strand of the target sequence of interest. Shown here, target-specific hybridization probe 1040a has a sequence that is complementary to the sense strand of the target of interest, which is included first PCR product 1025a, while target hybridization probe 1040b has a sequence that is complementary to the antisense strand of the same target of interest. As discussed herein, the target-specific hybridization probes may be joined to a moiety, such as biotin moiety 1045, to enable isolation (i.e., enrichment) of nucleic acid complexes, including the target sequence of interest, from a pool of non-target sequences. Further details of target-specific hybridization probe design are described herein. The single stranded PCR products including the target sequence of interest are isolated, i.e. enriched, from the pool of PCR products lacking this sequence by contacting the pool of single stranded PCR products with, e.g., streptavidin-coated beads, as described herein.

As shown in FIG. 10B, in step 4, the enriched PCR product strands are released from the target hybridization probe and the blocking oligonucleotides by treatment of the nucleic acid complexes with denaturing conditions (e.g., heat or 100 mM NaOH). The enriched PCR product strands are then subjected to a second round of PCR amplification using forward primers 1050a and reverse primers 1050b. In this embodiments, the forwards primer are joined at the 5′ end to biotin moiety 1050c, to produce a population of second PCR products 1050. (for the sake of simplicity, this exemplary illustration depicts the second PCR products resulting from amplification of the sense strand of double stranded second PCR product 1025b). The second PCR products include a biotin moiety joined to the 5′ end of one strand (i.e., the strand amplified with forward primer 1050a).

In step 5, second PCR products 1050 are purified by contacting the population of PCR products with streptavidin-coated beads 1055 to produce bead-bound second PCR products.

In step 6, the bead-bound second PCR products are A tailed while on support (i.e., bound to streptavidin-coated beads) to produce a single 3′ A overhang 1057, as disclosed herein.

In step 7, bead-bound PCR products 1050 are contacted with hairpin adapters and a DNA ligase enzyme under DNA ligation conditions. A single hairpin adapter 1060 is ligated to the free double stranded end of second PCR product 1050 to produce support-bound duplex template construct 1070.

In step 8, support-bound duplex template construct 1070 is contacted with a nickase endonuclease under nickase conditions. The nickase endonuclease cleaves nickase recognition site 1009 in the strand of the duplex template construct that is bound to streptavidin-coated bead 1055. Under suitable denaturing conditions, nicked duplex template construct 1080 is released from streptavidin bead 1055 and into solution. The free duplex template construct, including the enriched target sequence of interest, can then be used as a template for Xpandomer synthesis.

### Rolling Circle Amplification

In some instances, it may be desirable to generate a template construct for Xpandomer synthesis that includes a concatemer of covalently joined copies of a DNA target fragment. In this case, the copies of the template in the concatemer have at least a portion of the same nucleic acid sequence and can provide for redundant sequence reads of the template during Sequencing by Expansion.

In one embodiment, conventional rolling circle amplification (RCA) may be used to generated a concatemer template construct. RCA is well known in the art, see, e.g., U.S. Pat. No. 10,767,222, the contents of which are herein incorporated by reference in its entirety. Briefly, a population of single stranded DNA target fragment templates is provided, which are circularized by ligation. Primers are added, along with polymerase, dNTPs, buffers, etc., such that rolling circle amplification occurs to form concatemers (e.g., a “multimer”) of the DNA templates. In certain embodiments, the concatemers may be treated to synthesize the corresponding complementary strand, and then adapters may be added to make sequencing libraries.

According to some embodiments, polynucleotides among a plurality of polynucleotides from a sample are circularized. Circularization can include joining the 5′ end of a polynucleotide to the 3′ end of the same polynucleotide, to the 3′ end of another polynucleotide in the sample, or to the 3′ end of a polynucleotide from a different source (e.g., an artificial polynucleotide, such as an oligonucleotide adapter). In some embodiments, the 5′ end of a polynucleotide is joined to the 3′ end of the same polynucleotide (also referred to as “self-joining”). In some embodiment, conditions of the circularization reaction are selected to favor self-joining of polynucleotides within a particular range of lengths, so as to produce a population of circularized polynucleotides of a particular average length. For example, circularization reaction conditions may be selected to favor self-joining of polynucleotides from 150 to around 1000 nucleotides.

Rather than preferentially forming self-joining circularization products, one or more adapter oligonucleotides are used, such that the 5′ end and 3′ end of a polynucleotide in the sample are joined by way of one or more intervening adapter oligonucleotides to form a circular polynucleotide. For example, the 5′ end of a polynucleotide can be joined to the 3′ end of an adapter, and the 5′ end of the same adapter can be joined to the 3′ end of the same polynucleotide. An adapter oligonucleotide includes any oligonucleotide having a sequence, at least a portion of which is known, that can be joined to a sample polynucleotide

Where adapter oligonucleotides are used, the adapter oligonucleotides can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters, one or more restriction enzyme recognition sites, one or more overhangs complementary to one or more target polynucleotide overhangs

A variety of methods for circularizing polynucleotides are available. In some embodiments, circularization comprises an enzymatic reaction, such as use of a ligase (e.g., an RNA or DNA ligase). A variety of ligases are available, including, but not limited to, Circligase™ (Epicentre; Madison, Wis.), RNA ligase, T4 RNA Ligase 1 (ssRNA Ligase, which works on both DNA and RNA).

In some embodiments, prior to RCA, adapter with known sequences may be ligated to the single stranded DNA target fragments to facilitate circularization and/or provide hybridization sites for primers.

Circularization may be followed directly by one or more amplification reactions. In general, “amplification” refers to a process by which one or more copies are made of a target polynucleotide or a portion thereof. A variety of methods of amplifying polynucleotides (e.g., DNA and/or RNA) are available. Amplification may be linear, exponential, or involve both linear and exponential phases in a multi-phase amplification process.

In some embodiments, amplification comprises rolling circle amplification (RCA). A typical RCA reaction mixture comprises one or more primers, a polymerase, and dNTPs, and produces concatemers. Typically, the polymerase in an RCA reaction is a polymerase having strand-displacement activity. A variety of such polymerases are available, non-limiting examples of which include exonuclease minus DNA Polymerase I large (Klenow) Fragment, Phi29 DNA polymerase, Taq DNA Polymerase, Bst, Bsu or engineered variants thereof, and the like. In general, a concatemer is a polynucleotide amplification product comprising two or more copies of a target sequence from a template polynucleotide (e.g., about or more than about 2, 3, 4, 5, 6, 7, 8, 9, 10, or more copies of the target sequence; in some embodiments, about or more than about 2 copies). Amplification primers may be of any suitable length, such as about or at least about 5, 10, 15, 20, 25, 30, 35, 40, 45, 50, 55, 60, 65, 70, 75, 80, 90, 100, or more nucleotides, any portion or all of which may be complementary to the corresponding target sequence to which the primer hybridizes (e.g., about, or at least about 5, 10, 15, 20, 25, 30, 35, 40, 45, 50, or more nucleotides).

As discussed herein, in certain embodiments, the concatemer of DNA templates may be copied to generate a double stranded concatemer template construct; alternatively, the concatemer may be remain as a single stranded multiplexed template construct. In either case, the template construct may be processed to generate smaller concatemers, each including a reduced number of template copies (e.g., up to a single template copy) in the single construct molecules. In certain embodiments a processing step may include digestion with a restriction enzyme. Any of the multiplexed template constructs produced by RCA may be used for Xpandomer synthesis and Sequencing by Expansion, as disclosed herein.

Epigenetic Detection using Duplexed or Multiplexed Template Constructs

Advantageously, any of the duplexed template constructs for Xpandomer disclosed herein may be used for epigenetic analysis, e.g., for the detection of modified nucleobases in a DNA target fragment. Exemplary epigenetic modifications detectable by the disclosed methods include, but are not limited to, 5-methylcytosine (5-mC), 5-hydroxymethylcytosine (5-hmC), 5-carboxycytosine (5-caC), f5-ormylcytosine (5-fC), 8-oxo-7,8-dihyroguanine (oxoG), uracil, methyladenine (mA), and others.

In certain embodiments, “parent-daughter” duplex constructs may be synthesized under conditions in which the daughter strand copy of the parental template strand is synthesized with “native” dNTPS. In such embodiments, the parental template strand retains endogenous epigenetic information, while the daughter strand copy retains the genetic information of the parental target fragment, as it is synthesized in vitro with native dNPTs.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

The differential base modifications between daughter strands relative to the paired parental strand can be identified using any suitable conversion chemistry, or methodology, known in the art. For example, bisulfite sequencing has been an accepted standard for mapping methylomes. Sodium bisulfite chemically modifies unmethylated cytosines, causing their deamination to uracils. However, 5mC and 5hmC are not converted (see, e.g., Li, Y. and Tollefsbol, Methods Mol Biol. 2011; 719:11-21). Sequencing distinguishes cytosines from these modified forms as they are read as thymines and cytosines, respectively. Despite its widespread use, bisulfite sequencing has significant drawbacks. For example, it requires extreme temperatures and pH, which cause depyrimidination of DNA, resulting in DNA degradation. Furthermore, unmethylated cytosines are damaged disproportionately compared with 5mC or 5hmC, resulting in bisulfite libraries that have an unbalanced nucleotide composition. All these issues give rise to libraries with reduced mapping rates and skewed GC content representation.

An alternative epigenetic detection method is based on enzymatic conversion, are also known in the art, such as TET/APOBEC or EM-SEQ (see, e.g., Vaisvila, R. et al., Genome Res. 2021 July; 31(7): 1280-1289.). This method detects 5mC and 5hmC using two sets of enzymatic reactions. In the first reaction, TET2 and T4-BGT convert 5mC and 5hmC into products that cannot be deaminated by APOBEC3A. In the second reaction, APOBEC3A deaminates unmodified cytosines by converting them to uracils. Therefore, these three enzymes enable the identification of 5mC and 5hmC. As with the bisulfite method, these conversion chemistries lead to deamination of native cytosine to uracil, while certain modified forms of cytosine remain resistant. Thus, these enzymatic conversion options also reduce the complexity of the genome as native cytosine reads as uracil during a sequencing reaction.

In certain preferred embodiments, the epigenetic detection methods of the present invention using duplexed templates constructs employ an alternative chemo-enzymatic conversion strategy developed by the inventors, referred to herein as “abasic sequencing”, which is disclosed in PCT patent application no. PCT/EP2023/079149, filed Oct. 19, 2023, the contents of which are disclosed herein in their entirety.

FIG. 11A illustrates a simplified, non-limiting example of the chemo-enzymatic conversion of 5-mC to a uracil oxime mimetic. Here, a DNA target molecule including a 5-mC residue is treated with a TET enzyme (I) to convert 5-mC to 5-caC, and a TDG glycosylase (II) to excise the 5-caC nucleobase and generate an abasic site. In this example, the DNA target is also treated with an aminoxyalkyl uracil mimetic (III), which chemically reacts with the abasic site to form a stable oxime mimetic adduct (IV). In this embodiment, aminoxyalkyl uracil mimetic (III) is 1-[2-(aminooxy)ethyl]-uracil. Advantageously, the inventors have found that both the enzymatic conversion and excision of 5-mC with TET and TDG as well as the chemical conversion of the abasic nucleotide to the stable oxime adduct can be performed in a single reaction, i.e., a “one-pot” reaction. This one-pot reaction is also referred to herein as a “chemo-enzymatic nucleobase conversion reaction”. Importantly, the oxime mimetic adduct (IV) is capable of base-pairing with adenine and thus is read as uracil during DNA sequencing.

FIG. 11B illustrates a simplified, non-limiting example of how chemo-enzymatic conversion of 5-mC to the uracil oxime mimetic can be used in the detection of 5-mC in a duplexed DNA template of the present invention. Here, as discussed with reference to FIG. 11A, the 5-mC residues in the parental template strand are susceptible to steps (I) through (IV) that convert 5-mC to the uracil oxime mimetic. In contrast, the daughter strand copy of the duplex template is resistant to the conversion, as it incorporates native nucleotides, such that native G is incorporated into the daughter strand opposite positions of 5-mC in the parental template. For sequence comparison analysis, the duplexed template, including a converted parental strand covalently joined to an unconverted daughter strand by an intervening hairpin adapter serves as a template for the Sequencing by Expansion (SBX®) protocol (VI), as described further herein. The resulting sequencing reads of the daughter strand portion of the duplex template will indicate “G” at each of the positions of 5-mC in the daughter template, while the sequencing reads of the parent strand will indicate “A” at each of the positions of 5-mC in the parental template. Thus G:A mispairs in the sequence of the Xpandomer copy of the duplexed template reveal the positions of 5-mC in the original target fragment.

In other embodiments of the present invention, epigenetic detection methods may utilize the parent-parent duplexed template for Xpandomer synthesis. In some embodiments, prior to conversion, the parent-parent duplexed template may optionally be copied into a daughter-daughter duplexed template to provide a duplexed reference sequence encoding the genetic information of the parental template.

### Glycosylase-Mediated Excision of Modified Nucleobases

In one aspect, the methods of the present invention include the step of treating the duplex template constructs with a DNA glycosylase enzyme to specifically excise the modified base of interest. Many DNA glycosylases are known in the art, targeting a wide range of specifically modified nucleobases and DNA damage elements, including sequence mismatches and a large range of epigenetic modifications. Exemplary epigenetic modifications detectable by the described methods include, but are not limited to, 5-methylcytosine (5-mC), 5-hydroxymethylcytosine (5-hmC), 5-carboxycytosine (5-caC), f5-ormylcytosine (5-fC), 8-oxo-7,8-dihyroguanine (oxoG), uracil, methyladenine (mA), and others.

There are two main classes of DNA glycosylases: monofunctional and bifunctional. Monofunctional glycosylases have only glycosylase activity and cleave the N-glycosidic bond linking a damaged or modified nucleobase to the sugar-phosphate backbone of DNA. All DNA glycosylases cleave glycosidic bonds, but differ in their base substrate specificity and in their reaction mechanisms, Bifunctional glycosylases also possess apurinic or apyrimidinic site (AP) lyase activity that enables them to cut the phosphodiester bond of DNA at a base lesion, creating a single-strand break.

A non-limiting list of exemplary DNA glycosylases that are useful in the methods of the present invention are set forth in Table 2. In some instances, one or more of the DNA glycosylases listed in Table 2 may be used in the described methods to excise modified bases of interest from DNA target fragments. While select DNA glycosylases are specifically identified in this disclosure, it is understood that any suitable DNA glycosylase can be used in the performing the base excision step of the described methods.

TABLE 2
DNA Glycosylases

| Enzyme | Type | Species | Substrate(s) |
|---|---|---|---|
| N-methylpurine | Monofunctional | Human | 3-mA; 7-mG; 3-mG |
| DNA Glycosylase | DNA glycosylase | | |
| (MPG) | | | |
| MutY Homolog | Monofunctional | Human | A mis-paired with 8-oxoG, |
| (MUTYH) | DNA glycosylase | | C, or G |
| Nth-like DNA | Bifunctional DNA | Human | Thymine glycol (Tg); 2,6- |
| Glycosylase 1 | Glycosylase | | diamino-4-oxo-5- |
| (NTHL1) | | | formamidopyrimidine (FapyG); |
| | | | 5-hmC; 5-hydroxyuracil |
| | | | (5-HU) |
| Nei-like DNA | Bifunctional DNA | Human | Tg; FapyG; 4,6-diamino-5- |
| Glycosylase 1 | Glycosylase | | formamidopyrimidine (FapyA); |
| (NEIL1) | | | 8-oxoG; 5-HU |
| NEIL2 | Bifunctional DNA | Human | Tg; FapyG; FapyA; 8-oxoG; |
| | Glycosylase | | 5-HU |
| NEIL3 | Mono-or | Human | FapyG; FapyA |
| | bifunctional DNA | | |
| | glycosylase | | |
| 8-oxoguanine DNA | Mono-or | Human | 8-oxoG |
| glycosylase (OGG1) | bifunctional DNA | | |
| | glycosylase | | |
| Uracil DNA | Monofunctional | Human | U; 5-FU |
| Glycosylase 1 | DNA glycosylase | | |
| (Ung1) | | | |
| Uracil DNA | Monofunctional | Human | U, 5-FU |
| Glycosylase 2 | DNA glycosylase | | |
| (Ung2) | | | |
| Single-strand | Monofunctional | Human | 5-hmU; U mis-paired with G; |
| selective | DNA glycosylase | | 5-FU |
| monofunctional | | | |
| uracil glycosylase | | | |
| (SMUG1) | | | |
| Thymine DNA | Monofunctional | Human | U or T mis-paired with G; |
| Glycosylase (TDG) | DNA glycosylase | | 5-hmU; 5-fC, 5-CaC |
| Methyl binding | Monofunctional | Human | U or T mis-paired with G; |
| domain 4 (MBD4) | DNA glycosylase | | 5-hmU in CpG islands |
| Fpg | Bifunctional DNA | E. coli | 8-oxoG; 5-caC |
| | Glycosylase | | |
| Ung | Monofunctional | E. coli | U |
| | DNA glycosylase | | |
| Demeter (DME) | Bifunctional DNA | A. thaliana | 5-mC |
| | Glycosylase | | |
| ROS1 | Bifunctional DNA | A. thaliana | 5-mC; T mis-paired with G |
| | Glycosylase | | |

In one embodiment, the present methods utilize a DNA glycosylase that acts directly on 5-mC, i.e., a glycosylase that is capable of hydrolyzing the glycosidic bond between the 5-mC residue and the sugar-phosphate backbone. For example, a suitable DNA glycosylase that directly excises 5-inC may be a member of the DEMETER (DIVE) family of DNA glycosylases, e.g., DiVE, ROS1, or DMEL. The DME gene of Arabidopsis encodes a 1,729 amino acid protein with a centrally located DNA glycosylase domain (amino acids 1167-1368) that includes a helix-hairpin-helix (HhH) motif. The MTH motif in DME catalyzes excision of 5-inC (see, e.g., Choi et al., 2002. Cell 110:33-42). In certain embodiments, the DiVE glycosylase may be a variant that comprises amino acids 1167-1368 but lacks certain other regions of the protein.

In some instances, a suitable DNA glycosylase that acts directly on 5-mC may be an orthologue of DME. As used herein, the term “orthologue” means one of two or more homologous gene sequences found in different species.

In instances where the DNA glycosylase is a bifunctional enzyme, the glycosylase (e.g., DME, or an orthologue thereof), may be mutated to inactivate lyase activity, while still retaining glycosylase activity. The reaction mechanism of bifunctional DNA glycosylases is well known in the art (see, e.g., Scharer and Jiricny. 2001. Bioessays 23: 270-281). In some cases, a conserved aspartic acid acquires a proton from a conserved lysine residue that attacks the C1′ carbon of the deoxyribose ring, creating a covalent DNA-enzyme intermediate. Beta or gamma elimination reactions release the enzyme from the DNA and cleave one of the phosphodiester bonds. Mutant forms of DME in which the invariant aspartic acid at position 1304 or the lysine at position 1286 have been altered (e.g., variants D1304N or K1286Q) been shown to reduce DNA glycosylase activity while preserving enzyme structure and stability (see, e.g., Fromme et al. 2004 Nature 427: 652-656).

Other mutations that inactivate or optimize suitable features of the DNA glycosylase are also contemplated by the present invention. For example, the DNA glycosylase may be engineered to increase its stability and/or solubility. The DNA glycosylase may also be engineered to optimize for a desired substrate specificity.

In certain embodiments, thymine DNA glycosylase (TDG) may be used to excise its known targets, 5-carboxycytosine (5-caC) and 5-formylcytosine (5-fC). In further embodiments, TDG may be used to identify 5-methylcytosine (5-mC) and 5-hydroxymethylcytosine (5-hmC), which are modified bases that it does not specifically recognize. For example, DNA target fragments may also be treated with a ten eleven translocation (TET) enzyme prior to treatment with TDG. The TET family proteins included three human proteins (TET1, TET2, and TET3) and are cytosine oxygenases that catalyze the conversion of 5-methylcytosine (5-mC) into 5-hydroxymethylcytosine (5-hmC). 5-hmC can be further oxidized into 5-formylcytosine (5-fC) and 5-carboxylcytosine (5-caC) by TET proteins (see, e.g., Parker, et. al. 2019. Biochemistry 58: 450-467). In another instance, a suitable TET enzyme may be any TET orthologue, e.g., ngTET, isolated from Naegleria (see, e.g., Hashimoto, et. al. 2014. Nature 506(7488): 391-395). Thus, in certain embodiments, TDG may be used to excise any existing 5-caC and 5-fC modified bases present in a DNA target fragment also treated with a TET enzyme.

Other comparable methods for altering the selective excision of modified bases are possible according to the present invention. For example, a similar method may be performed to detect the same bases discussed above using thymine DNA glycosylase (TDG) and uracil DNA glycosylase (UDG).

### Stabilization of Abasic Sites in DNA Target Fragments

Advantageously, according to the methods of the present invention, abasic sites generated in DNA target fragments may be protected from further degradation with a stabilizing agent. In certain embodiments, a suitable stabilizing agent may be a chemical that covalently binds to the abasic site to form a stable abasic adduct. Certain aldehyde-reactive compounds are known to react with the open-ring aldehyde form of the abasic site to create stable open structures, that are referred to herein abasic adducts. Abasic adducts are refractory to enzymatic activity (e.g., lyase-mediated degradation) or to degradation-inducing chemical conditions, such as high pH. Some exemplary, non-limiting, structural classes of aldehyde-reactive stabilizing agents are described below. Each class varies in reaction rates, stability, and size of the resulting protected adduct product. The chemical properties of each abasic adduct product provide different chemoenzymatic properties with regard to duration of stabilization and suitability as a template for extension by a DNA polymerase.

In one embodiment, suitable stabilizing agents may be from the group of O-hydroxylamines (compound IIIa), which are a class of compounds known to react with the aldehydic group of the open-ring form of the abasic site to create very stable oxime structures that are refractory to β-elimination by enzymatic activity (e.g., AP or dRp lyases) or by high pH.

### Aminoxyalkyl Nucleobase Mimetics

As discussed herein, and with reference to FIG. 6, certain chemical stabilizing agents react with abasic sites in DNA to form a stable oxime adduct that prevents subsequent degradation of the phosphodiester backbone. As used herein, the term “oxime” refers to an organic compound belonging to the imines, with the general formula, RR′C═N—OH, where R is an organic side chain and R′ may be hydrogen, forming an aldoxime, or another organic group, forming a ketoxime. O-substituted oximes form a closely related family of compounds. One particularly useful class of stabilizing agents used to form oxime adducts are those with the generalized aminoxyalkyl structure, H₂N—O—R, as disclosed herein. Advantageously, the inventors have discovered that certain oximes have the further capability to biologically mimic the Watson-Crick base-pairing activity of natural nucleobases. Thus, they not only stabilize abasic sites, but also direct incorporation of specific nucleotides at opposing sites during daughter strand synthesis. Such aminoxyalkyl-based stabilizing reagents and their corresponding oxime adduct products may be referred to in certain embodiments herein alternatively as, “nucleobase mimetics”, “aminoxyalkyl nucleobase mimetics”, or “nucleobase oxime mimetics.”

In one embodiment, the uracil mimetic, 1-[2-(amino)ethyl]-uracil, is used to stabilize abasic sites, as the aminoxyalkyl constituent of the mimetic compound reacts with the abasic site to form a stable oxime adduct. Advantageously, the heterocycle constituent of the compound is able to from Watson-Crick base pairs with adenine and will thus direct incorporation of dATP during daughter strand synthesis.

Other exemplary aminoxyalkyl nucleobase mimetics suitable for the methods of the present invention include 1-[3-(aminoxy)propyl]-uracil, 1-[4-(aminoxy)butyl]-uracil, 1-[5-(aminoxy)pentyl]-uracil, commercially available from, e.g., Enamine Ltd. In other embodiments, the present invention contemplates new aminoxyalkyl nucleobase mimetics in which certain chemical features are optimized for particular applications. For example, mimetics may include heterocycles other than uracil, such as thymine, cytosine, guanine, or adenine. In other embodiments, the mimetics may include alternative atomic distances between the oxime and the heterocycle, e.g., from two carbons to three, four, or five carbons. Certain exemplary aminoxyalkyl nucleobase mimetics include the following: 1-[2-(aminoxy)ethyl]-2,4-diiodo-5-methyl benzene, compound (A); 1-[2-(aminoxy)ethyl]-2,4-dibromo-5-methyl benzene, compound (B); 1-[2-(aminoxy)ethyl]-2,4-dichloro-5-methyl benzene, compound (C); 1-[2-(aminoxy)ethyl]-2,4-difluoro-5-methyl benzene, compound (D); 1-[2-(aminoxy)ethyl]-thymine, compound (E); and further prophetic pseudo uridine analogs, compounds (F) and (G).

### Solid-Phase Synthesis

In certain embodiments, one or more steps of generating the duplexed template constructs and/or Xpandomer synthesis may be conducted on a solid support. As used herein, the terms “solid support”, “solid-state”, “solid-phase”, and “substrate” may be used interchangeably and refer to a material or group of materials having a rigid or semi-rigid surface or surfaces. In many embodiments, at least one surface of the solid support will be substantially flat, e.g., a surface of a polymeric microfluidic card or chip. In some embodiments it may be desirable to physically separate regions of a card or chip for different reactions with, for example, etched channels, trenches, wells, raised regions, pins, or the like. According to other embodiments, the solid support(s) will take the form of insoluble beads, resins, gels, membranes, microspheres, or other geometric configurations composed of, e.g., controlled pore glass (CPG) and/or polystyrene.

The invention encompasses solid-phase synthesis methods in which a capture moiety is immobilized on a solid support. In certain instances, the capture moiety includes a first end covalently bound to the solid support and a second end that provides a functional group capable of binding to the 5′ end of a single stranded target sequence. As used herein, the term “immobilized”, refers to the association, attachment, or binding between a molecule (e.g., linker, adapter, or oligonucleotide) and a support in a manner that provides a stable association under the conditions of elongation, amplification, ligation, and other processes as described herein. Such binding can be covalent or non-covalent. Non-covalent binding includes electrostatic, hydrophilic and hydrophobic interactions. Covalent binding is the formation of covalent bonds that are characterized by sharing of pairs of electrons between atoms. Such covalent binding can be directly between the molecule and the support or can be formed by a cross linker or by inclusion of a specific reactive group on either the support or the molecule or both. Covalent attachment of a molecule can be achieved using a binding partner, such as avidin or streptavidin, immobilized to the support and the non-covalent binding of the biotinylated molecule to the avidin or streptavidin. Immobilization may also involve a combination of covalent and non-covalent interactions.

Any suitable covalent attachment means known in the art may be used for these purposes. The chosen attachment chemistry will depend on the nature of the solid support and any derivatization or functionalities applied thereto. The extension oligonucleotide may include a moiety, which may be a non-nucleotide chemical modification, to facilitate attachment. Certain exemplary embodiments of suitable surface chemistries include conventional streptavidin/biotin interaction chemistry and involve functionalization of a solid support, e.g., with a linker moiety that includes terminal a biotin moiety. In this embodiment, the 5′ end of single stranded DNA fragment (or oligonucleotide) is bound to the linker moiety. Attachment is mediated by a streptavidin moiety provided by the 5′ end of the single stranded DNA fragment. The linker moieties disclosed herein may be of sufficient length to connect the single stranded DNA fragment to the support such that the support does not significantly interfere with primer extension reaction.

Alternatively, immobilization of a capture moiety or oligonucleotide (e.g., an extension oligonucleotide) to a solid support may be accomplished by covalent linkage of the capture oligonucleotide to the solid support via a click reaction. In this embodiment, the covalent linkage may be mediated by a maleimide-PEG-alkyne linker that is crosslinked to the solid support. An alkyne moiety provided by the end of the linker distal to the substrate is capable of reacting with an azide group provided by the 5′ end of the capture oligonucleotide. Methods of functionalizing a solid support with maleimide-linker polymers is provided in Applicant's published Patent Application No. WO2020/172479, which is herein incorporated by reference in its entirety.

In certain instances, the linkage between the capture moiety and the solid support is cleavable, enabling primer extension products to be released from the support following synthesis. Cleavable linkers and methods of cleaving such linkers are known and can be employed in the provided methods using the knowledge of those of skill in the art. For example, the cleavable linker can be cleaved by an enzyme, a catalyst, a chemical compound, temperature, electromagnetic radiation or light. Optionally, the cleavable linker includes a moiety hydrolysable by beta-elimination, a moiety cleavable by acid hydrolysis, an enzymatically cleavable moiety, or a photo-cleavable moiety. In some embodiments, a suitable cleavable moiety is a photocleavable (PC) spacer or linker phosphoramidite available from Glen Research.

### Sequencing by Expansion

One nucleic acid sequencing methodology that may be implemented with the present invention is “Sequencing by Expansion” (SBX®), developed by Stratos Genomics (see, e.g., Kokoris et al., U.S. Pat. No. 7,939,259, “High Throughput Nucleic Acid Sequencing by Expansion”, which is herein incorporated by reference in its entirety). As discussed, SBX® uses biochemical polymerization to transcribe the sequence of a DNA template, e.g., a duplexed template construct, onto a measurable polymer called an “Xpandomer”. SBX® is based on the polymerization of highly modified, non-natural nucleotide analogs, referred to as “XNTPs”. XNTPs are expandable, 5′ triphosphate modified non-natural nucleotide analogs compatible with template dependent enzymatic polymerization. The XNTP has two distinct functional regions; namely, a selectively cleavable phosphoramidate bond, linking the 5′ α-phosphate to the nucleobase, and a symmetrically synthesized reporter tether (SSRT) that is attached within the nucleoside triphosphoramidate at positions that allow for controlled expansion by cleavage of the phosphoramidate bond. XNTPs are described in further details in Applicant's U.S. Pat. Nos. 10,301,345 and 10,774,105, which are herein incorporated by reference in their entireties. The SSRT includes linkers separated by the selectively cleavable phosphoramidate bond. Each linker attaches to one end of a reporter code. XNTP substrates incorporated into daughter strand products of template-dependent polymerization are in the “constrained” configuration. The constrained configuration of polymerized XNTPs is the precursor to the expanded configuration, as found in Xpandomer products.

The transition from the constrained configuration to an expanded configuration results from cleavage of the selectively cleavable phosphoramidate bonds within the primary backbone of the daughter strand. In this embodiment, the SSRTs include one or more reporters or reporter codes, specific for the nucleobase to which they are linked, thereby encoding the sequence information of the template. In this manner, the SSRTs provide a means to expand the length of the Xpandomer and lower the linear density of the sequence information of the parent strand.

The SSRT (i.e., “tether”) of the XNTP includes several distinct functional elements, or features, such as polymerase enhancement regions, reporter codes, and translation control element (TCEs). These features are discussed in further details in Applicant's published PCT application WO2020/236526, which is herein incorporated by reference in its entirety. Each of these features performs a unique function during translocation of the Xpandomer through a nanopore to produce a series of unique and reproducible electronic signal. The SSRT is designed for controlling the rate of Xpandomer translocation by the TCE through a combination of sterics and/or electrorepulsion, Different reporter codes are sized to block ion flow through a nanopore at different measurable levels. In certain embodiments, reference is made to the “reporter construct” of the XNTP, which includes, from a proximal end to a distal end, the TCE, a symmetrical Y brancher, and two symmetric reporter code, each joined to an end of the Y brancher distal to the TCE. The reporter construct is a feature of the larger SSR structure.

Specific SSRT polymeric sequences can be efficiently synthesized using phosphoramidite chemistry typically used for oligonucleotide synthesis. Reporter codes and other features can be designed by selecting a sequence of specific phosphoramidites from commercially available and/or proprietary libraries. Such libraries include, but are not limited to, polyethylene glycol with lengths of 1 to 12 or more ethylene glycol units and aliphatic polymers with lengths of 1 to 12 or more carbon units. In certain embodiments, the SSRTs include features referred to as “polymerase enhancement regions” at the ends of the SSRTs proximal to the nucleotide triphosphoramidate diester. Polymerase enhancement regions may include positively charged polyamine spacers (e.g., primary, secondary, tertiary, or quaternary amines) or triamine spacers (three secondary amines each separated by three carbons) that facilitate incorporation of XNTP structures by a nucleic acid polymerase. In certain embodiments, the polymerase enhancement region includes two repeat units spermine

As used throughout the present disclosure, the terms “linker A” and “linker B” refer to the regions of the SSRT that each include a polymerase enhancing region and one or more translocation deceleration features or regions, and, in certain embodiments, a spacer region that includes a polymer of, e.g., PEG6, which can be customized to modulate the length of the SSRT traversed in a nanopore.

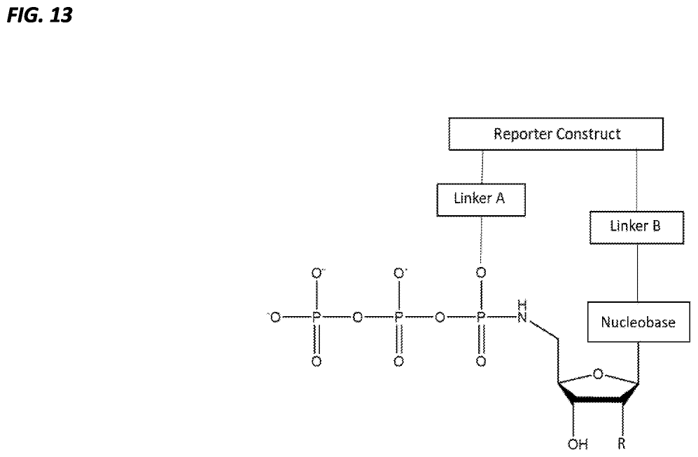

In certain embodiments, an XNTP may be a compound having the generalized structure depicted in FIG. 13. In one embodiment, R may be H, for example, when the compounds are used to sequence a DNA template. In certain embodiments, nucleobase is adenine, cytosine, guanine, thymine, uracil or a nucleobase analog. As one of skill in the art will appreciate, adenine, cytosine, guanine, thymine, and uracil are naturally occurring nucleobases. As used herein, the term “nucleobase analog” refers to non-naturally occurring nucleobases that are capable of forming Watson and Crick base pair with a complementary nucleobase on an adjacent single-stranded nucleic acid template.

To obtain sequence information, an Xpandomer is translocated through a nanopore, from the cis reservoir to the trans reservoir. As the Xpandomer translocates, a reporter enters the stem until its translocation control element stops at the stem entrance. The reporter is held in the stem until the TCE is enabled to pass into and through the stem, whereupon translocation proceeds to the next reporter. Upon passage through the nanopore, each of the reporter codes of the linearized Xpandomer generates a distinct and reproducible electronic signal, specific for the nucleobase to which it is linked.

In certain embodiments, Xpandomers produced by the SBX chemistry may be analyzed using a nanopore-based sequencing chip. A nanopore-based sequencing chip can incorporate a large number of sensor cells configured as an array. For example, the chip may include an array of one million cells configured in 1000 rows by 1000 columns of cells. Each cell in the array may include a control circuit integrated on a silicon substrate. Such nanopore-based sequencing chips, devices, and systems are described, e.g., in Applicant's published patent application no. WO2021/219795, which is herein incorporated by reference in its entirety.

Proprietary in-house bioinformatics pipelines are typically used to process sequencing reads. The methods disclosed herein leverage UMIs to enable pairing of related sequence reads. Read pairs may be quality filtered and trimmed of adapter and primer sequences. UMI sequences may be clustered together, defining UMI-families (all reads originating from a single DNA template).

### Xpandomer Synthesis Reaction

The Xpandomer synthesis reaction represent a critical step in SBX®, as it is responsible for accurately transcribing the sequence of the DNA template of interest into the sequence of the Xpandomer, which is the polymer directly read by the nanopore sensor. Through trial and error, the inventors have developed a complex reaction mixture for Xpandomer synthesis, which includes a DNA polymerase and many additives that enable incorporation of the bulky XNTP substrates by the polymerase into the very large Xpandomer macromolecule.

In certain embodiments, a non-limiting Xpandomer synthesis reaction mixture may include the following reagents: a buffer/salt system, polymerase cofactors, polymerase enhancing moieties (PEMs), a DNA polymerase, XNTP substrates, a phosphate shield molecule, a solvent, a crowding agent, and optionally, additional additives. In some embodiments, the buffer/salt system may include TrisCl and NaCl; the polymerase cofactors may include MnCl₂ formulated in MES; the PEMs may include molecules disclosed in Applicant's published PCT applications, WO2019/135975 and WO2020/263703, which are herein incorporated by reference in their entireties; the DNA polymerase may include a variant of DPO4 polymerase as disclosed in Applicant's U.S. Pat. Nos. 11,299,725, 11,708,566, 11,530,392 and U.S. provisional patent application No. 63/591,165, filed Oct. 18, 2023, each of which is herein incorporated by reference in their entireties; the phosphate shield molecule may include hexametaphosphate (HMP); the solvent may include NMP and DMSO; the crowding agent may include PEG8k; and the additional additives may include imidazole and betaine.

In certain embodiments, the Xpandomer synthesis reaction comprises a variant of wildtype DPO4 polymerase, designated C7326, with the following amino acid substitutions: F37T_D39L_K56Y_A57S_I59M_E63R_M76W_K78E_E79P_Q82W_Q83G_S86E_K152A_I153V_A155G_D156S_M157K_D179N_P184Q_G187P_N188Y_I189F_E192Q_1248T_S272C_V289W_T290R_E291S_D292R_L293W_D294N_I295S_V296Q_S297Y_G299W_R300S_T301W_K321Q_E324K_E325K_E327KΔ341-352 (SEQ ID NO:2). The amino acid sequence of wildtype DPO4 polymerase is set forth in SEQ ID NO:1, while the amino acid sequence of variant C7326 is set forth in SEQ ID NO:2. In some embodiments, a variant of DPO4 polymerase suitable for the practice of the present invention may be a variant that is at least 85% identical to SEQ ID NO:2.

One of the challenges encountered by the DNA polymerase when replicating the duplex template constructs of the present invention are double stranded regions formed when the two complementary strands of the target fragment are hybridized. DPO4 polymerase does not possess robust strand displacement activity and, as such, it does not efficiently extend through double stranded regions in a template. Indeed, the inventors have observed that the DPO4 variants commonly used in Xpandomer synthesis will prematurely “jump” from one template strand to the other as the duplex template construct is copied, thereby synthesizing incomplete Xpandomer copies of the two template strands. This phenomenon is referred herein to as a polymerase “U-turn”.

In optimizing Xpandomer synthesis conditions using the duplex template constructs of the present invention, the inventors have tested numerous biological additives and other physical forces or manipulations to reduce the occurrence of polymerase U-turns and increase the percentage of full-length duplexed Xpandomers synthesized. The following classes of additives were observed to reduce the rate of polymerase U turns during replication of duplexed template constructs: 1) single stranded binding proteins (SSBs), which are proteins that bind to and help stabilize single stranded regions of DNA and prevent formation of more stable secondary structures; 2) proteins, or enzymes, known to participate in DNA recombination processes, or to otherwise manipulate regions of single stranded DNA; 3) non-protein additives known to beneficially impact Xpanodmer synthesis (e.g., PEMs or polyphosphate analogs); 4) the biochemistry conditions of the Xpandomer synthesis reaction, including order of addition (e.g., pre-treatment of the template with an additive such as SSB prior to the Xpandomer synthesis reaction); 4) stretching forces proposed to enhance solid-state replication of duplexed template constructs. For example, in certain embodiments a duplex template construct may be associated with a solid support through hybridization of a sequence in its 3′ end with an extension oligonucleotide that is covalently bound to the support. The 3′ end of the extension oligonucleotide provides an initiation site for Xpandomer synthesis by a DNA polymerase, as disclosed herein. A blocker oligonucleotide can be designed to hybridize to a sequence in the opposite 5′ end of the duplex template construct. In certain embodiments, the 3′ end of the blocker oligonucleotide can be joined to a moiety that is susceptible to, e.g., an applied external force that “stretches” apart double stranded regions by overcoming the strength of the hydrogen bonds between the two complementary strands of the duplex template construct.

A non-limiting list of SBX® synthesis enhancers according to the present invention is set forth in Table 3. It is to be emphasized that the following list of exemplary additives is intended to merely illustrate one of many suitable possibilities of the larger genus (i.e., class) of additives, and other forces recited in Table 3, contemplated by the present invention.

TABLE 3
SBX ® synthesis enhancers

| Additive | Class |
|---|---|
| TTH (Thermus thermophilus) | Protein (SSB) |
| SEQ ID NO: 3 | |
| KOD (Thermococcus kodakarenis) | Protein (SSB) |
| SEQ ID NO: 4 | |
| Gp32 (Bacteriophage T4) | Protein (SSB) |
| SEQ ID NO: 5 | |
| SSB (Escherichia coli) | Protein (SSB) |
| SEQ ID NO: 6 | |
| RPA (Homo sapiens) | Protein (SSB) |
| SEQ ID NO: 7 | |
| NCp7 (HIV-1) | Protein (SSB) |
| SEQ ID NO: 8 | |
| RecA (Escherichia coli) | Protein (SSB) |
| SEQ ID NO: 9 | |
| Helicase (Escherichia coli UvrD) | Protein (enzyme) |
| SEQ ID NO: 10 | |

| | |
|---|---|
| CS-43,43 PEM 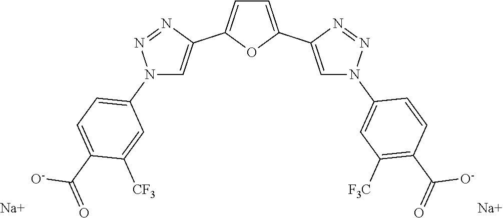 [SMILES: [Na+] ; O=C([O-])c1ccc(-n2cc(-c3ccc(-c4cn(-c5ccc(C(=O)[O-])c(C(F)(F)F)c5)nn4)o3)nn2)cc1C(F)(F)F ; [Na+]] | Non-protein additive 4,4-(furan-2,5-diylbis(1H-1,2,3-triazole 4,1-diyl))bis(2-(trifluoromethyl)benzoic acid |
| AZ-43,43 PEM 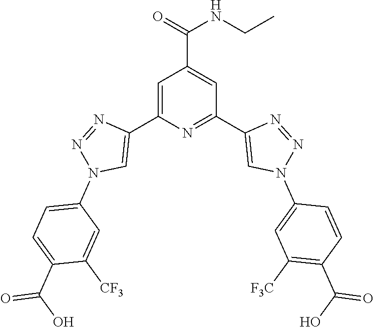 [SMILES: CCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] | non-protein additive 4,4′-((4-ethylcarbamoyl)pyridine-2,6- diyl)bis(1H-1,2,3-triazole-4,1-diyl))bis(2- trifluoromethyl)benzoic acid) |
| DF-99,99 PEM 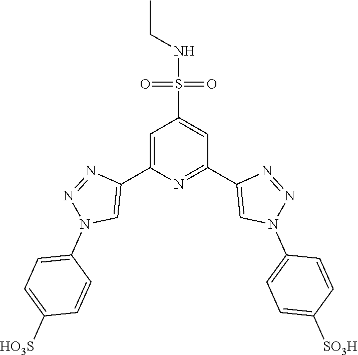 [SMILES: CCNS(=O)(=O)c1cc(-c2cn(-c3ccc(S(=O)(=O)O)cc3)nn2)nc(-c2cn(-c3ccc(S(=O)(=O)O)cc3)nn2)c1] | non-protein additive p-{4-[4-(Ethylaminosulfonyl)-6-[1-(p- sulfophenyl)-1H-1,2,3-triazol-4-yl]-2- pyridyl]-1H-1,2,3-triazol-1- yl}benzenesulfonic acid |
| Acetamide | biochemistry condition (solvent) |
| NMP:DMSO | biochemistry condition (solvent) |
| Brownian motion | Stretching force |
| Hydrodynamic | Stretching force |
| Magnetic | Stretching force |
| Acoustic | Stretching force |
| Electrophoretic | Stretching force |
| Electroosmotic | Stretching force |
| Photonic | Stretching force |

### Diagnostic and Prognostic Methods

In particular embodiments, the methods can be directed to diagnosing an individual with a condition that is characterized by a methylation level and/or pattern of methylation at particular loci in a test sample that are distinct from the methylation level and/or pattern of methylation for the same loci in a sample that is considered normal or for which the condition is considered to be absent. The methods can also be used for predicting the susceptibility of an individual to a condition that is characterized by a level and/or pattern of methylated loci that is distinct from the level and/or pattern of methylated loci exhibited in the absence of the condition.

With particular regards to cancer, changes in DNA methylation have been recognize as one of the most common molecular alterations in human neoplasia. Hypermethylation of CpG islands located in promoter regions of tumor suppressor genes is a well-established and common mechanism for gene inactivation in cancer (Esteller, Oncogene 21(35): 5427-40 (2002)). In contrast, a global hypomethylation of genomic DNA is observed in tumor cells; and a correlation between hypomethylation and increased gene expression has been reported for many oncogenes (Feinberg, Nature 301(5895): 89-92 (1983), Hanada, et al., Blood 82(6): 1820-8 (1993)). Cancer diagnosis or prognosis can be made in a method set forth herein based on the methylation state of particular sequence regions of a gene including, but not limited to, the coding sequence, the 5′-regulatory regions, or other regulatory regions that influence transcription efficiency.

A reference genomic DNA (for example, gDNA considered “normal”) and a test genomic DNA that are to be compared in a diagnostic or prognostic method, can be obtained from different individuals, from different tissues, and/or from different cell types. In particular embodiments, the genomic DNA samples to be compared can be from the same individual but from different tissues or different cell types, or from tissues or cell types that are differentially affected by a disease or condition. Similarly, the genomic DNA samples to be compared can be from the same tissue or the same cell type, wherein the cells or tissues are differentially affected by a disease or condition.

### Compositions and Kits

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system comprising two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

In certain embodiments, presented herein are compositions for conducting a method described herein, and including one or more of the elements thereof. In some embodiments, a composition comprises a “parent-parent” duplex nucleic acid template comprising a double stranded nucleic acid target fragment with a hairpin adapter covalently joined to a first end and a hairpin adapter joined to a second end. In this embodiment, both of the strands of the target fragment are derived from the original sample of nucleic acids (e.g., both strand were synthesized in vivo).

In other embodiments, a composition comprises a “parent-daughter” duplex nucleic acid template comprising a double stranded nucleic acid target fragment with a hairpin adapter covalently joined to a first end and a hairpin adapter joined to a second end. In this embodiment, one of the strands of the target fragment is derived from the original sample of nucleic acids (e.g., in vivo) and the other strand is a newly synthesized daughter strand (e.g., synthesized in vitro).

In some embodiments, the compositions may further include a primer hybridized to a single stranded arm of the Y adapter of a duplex nucleic acid template. In certain embodiments, the primer may be attached to a substrate. In some embodiments, the substrate is a polymer coated surface of a flow cell.

In certain embodiments, presented herein is a library preparation kit for producing a duplex template construct in accordance with any of the methods described herein and including one or more elements thereof. In embodiments, the kit comprises: a first adapter, in which the first adapter is a hairpin adapter; and a second adapter, in which the second adapter is a Y adapter and one or more of a nucleic acid fragmentation mixture, a ligation mixture including the adapters, an exonuclease digestion mixture, a nucleic acid purification mixture, and a nucleic acid quantification mixture that may, in certain embodiments, include a molecular beacon hairpin adapter.

In certain embodiments, presented herein is a sequencing kit for sequencing double stranded nucleic acids, in accordance with any of the methods disclosed herein and including one or more elements thereof. In embodiments, the kit includes all the reagents necessary for SBX®, including Xpandomer synthesis, processing, and elution.

Adapters, primers, and nucleic acid template constructs may be supplied in the kits ready for use, or alternatively, as concentrates requiring dilution before use, or even in a lyophilized or dried form requiring reconstitution prior to use. If required, the kits may further include a supply of a suitable diluent for dilution or reconstitution of the nucleic acids. The reagents for the Xpandomer synthesis reaction may, in certain embodiments, be provided as a pre-mixed “master mix” that can be stored at four degrees Celsius, −20 degrees Celsius, or −80 degrees Celsius until use. Suitable formulations of SBX® master mixes are disclosed, e.g., in Applicant's co-pending provisional patent application No. 63/688,811, filed Aug. 30, 2024 and entitled, “Compositions for Replicating a Nucleic Acid”, the entire contents of which are herein incorporated by reference in its entirety.

### EXAMPLES

### Example 1

### Solid-State Synthesis of a “Parent-Parent” Duplex Template Construct

In this example, a parent-parent duplex template construct was synthesized on a solid support. The support was provided by streptavidin-coated magnetic beads to which a Y adapter was bound and immobilized for assembling the duplex construct. An overview of this method is illustrated in FIG. 4.

Preparation of Y adapter-bound beads. Dynabeads MyOne Streptavidin C1 paramagnetic beads were commercially obtained from ThermoFisher Scientific. For this experiment, 30 μL of bead suspension was pipetted into a tube and captured with a magnet. The supernatant was discarded and the beads were resuspended in 120 μL of binding buffer+TWEEN-20 (1M NaCl; 10 mM TrisCl, pH 8.0; 1 mM EDTA; 0.1% TWEEN-20). The beads were captured with a magnet and the supernatant was discarded. The beads were resuspended in 120 μL of 100 mM NaOH and incubated for 5 minutes at room temperature. The beads were captured with a magnet, the supernatant was discarded and the beads were washed twice with 120 μL of binding buffer+TWEEN-20. The beads were resuspended in 298.50 μL of binding buffer+TWEEN-20.

Oligonucleotide binding reaction. The 5′ oligonucleotide strand of the Y adapter (i.e., the strand that contributes the 5′ single stranded arm in the final adapter structure), including a biotin moiety at the 5′ end joined to the oligonucleotide by a linker, was prepared by conventional oligonucleotide synthesis technology. In this experiment the 5′ oligonucleotide strand included the following features, from 5′ to 3′: two biotin moieties, a repeat of three PEG6 spacers, a repeat of five deoxyuridines (for cleavage by the UDG enzyme), a 32mer oligonucleotide sequence that forms the 5′ single stranded arm of the final Y adapter and includes an extension oligonucleotide hybridization sequence, a 27mer oligonucleotide sequence that forms the double stranded stem portion of the final adapter structure when hybridized to the 3′ oligonucleotide strand, and a terminal deoxythymidine. The biotinylated 5′ oligonucleotide strand was bound to the streptavidin-coated beads by adding 30 μm of oligonucleotide to the 298.50 μL bead suspension. The sample was incubated on a rotator for 15 minutes. The beads were then captured with a magnet and the supernatant was removed. The beads were washed three times with 300 μL of binding buffer+TWEEN-20 and moved to a fresh tube for the final wash. The beads were resuspended in 30 μL of pre-ligation buffer (100 mM NaCl; 10 mM TrisCl, pH 8.0; 0.1% TWEEN-20).

On-bead Y adapter hybridization. A sample of the 3′ oligonucleotide strand of the Y adapter (i.e., the strand that contributes the 3′ single stranded arm in the final adapter structure) was provided that included a sequence at the 5′ end that is complementary to the 27mer oligonucleotide sequence of the 5′ oligonucleotide that forms the double stranded stem portion of the final adapter structure. A sample containing 2.1 pmol of the 3′ oligonucleotide strand was added to the bead-bound 5′ oligonucleotide strand of the Y adapter. A hybridization reaction was carried out in a thermocycler in which the temperature of sample was ramped up to 72 degrees C for one minute then ramped down to 55 degrees C for one minute and stored at 4 degrees C.

Preparation of hairpin adapter for ligation. A sample of hairpin adapter was provided in which the adapter included a single T overhang at the 3′ end. The hairpin adapter was formed from an oligonucleotide that had the following features: a first 12mer oligonucleotide sequence that forms the double stranded stem portion of the hairpin adapter, a 7mer oligonucleotide sequence that forms the single stranded loop of the hairpin adapter, a second 12mer oligonucleotide with a sequence complementary to that of the first 12mer oligonucleotide and a final deoxythymidine residue. The 5′ end of the adapter was phosphorylated in a PNK reaction that included 20 pmol hairpin adapter, 10 mM ATP and 1 U/μL PNK enzyme in PNK buffer. The reaction was run for 30 minutes at 37 degrees C followed by 72 degrees C for 2 minutes and 55 degrees C for one minute.

Preparation of library fragments for ligation—ERAT. An ERAT master mix was prepared that included ERAT enzyme and ERAT buffer using the commercially KAPA Hyper Prep kit, available from Roche Sequencing Solutions, Inc. according to the manufacturer's instructions for use. A sample including 10 ng of target fragment DNA (i.e., fragmented human genomic DNA) was added to 60 μL of ERAT master mix and the sample was incubated at 20 degrees C for 30 minutes followed by 65 degrees C for 30 minutes.

First Ligation Reaction (ligation of hairpin adapter to DNA target fragments in solution). A ligation master mix was prepared that included 0.27 U/μL of PNK enzyme; 1.36 U/μL of deadenylase enzyme and 0.09 U/μL of Codexis DNA ligase enzyme in HyperPrep ligation buffer. A 350 μL ligation reaction was prepared that included 0.45 ng/μL of DNA target fragment, 0.01p M of hairpin adapter in 1× ligation master mix. The reaction was incubated at 16 degrees C for 30 minutes then quenched by adding 5 μL of proteinase K enzyme and incubating at 55 degrees C for 15 minutes. The reaction was then incubated at 95 degrees C for four minutes to denature the target fragment-hairpin adapter ligation products.

Size-selective nucleic acid purification (i.e., SPRI clean up). KAPA Pure Beads were commercially obtained from Roche Sequencing Solutions, Inc. and added to the first ligation reaction according to the manufacturer's instructions for use. The sample was incubated at room temperature for 10 minutes. The beads were collected on a magnet, the supernatant was discarded and the beads were washed twice in 80% ethanol and allowed to air dry for three to five minutes. An elution buffer comprised of water was added to the beads followed by incubation for five minutes. The beads were pelleted and the supernatant containing the eluted ligation products (e.g., a sample of nucleic acids greater than 150 bp) was collected and retained for use in the second ligation reaction.

Second ligation reaction (ligation of hairpin-ligated DNA target fragments to Y adapter on support). A second ligation master mix was prepared that included 0.1% TWEEN-20, 0.27 U/μL PNK enzyme, 1.36 U/μL deadenylase enzyme, and 0.09 U/μL Codexis DNA ligase enzyme in HyperPrep ligation buffer. 50 μL of YAD hybridization beads was pelleted and the supernatant was discarded. 50 μL of the first ligation reaction (containing the DNA target fragments ligated to the hairpin adapter) was added to the YAD-bound beads followed by 60 μL of the second ligation master mix. The reaction was subjected to thermoshaking at 2000 rpm for one hour at 23 degrees C. 5 μL of EDTA was then spiked-in. The reaction was then quenched by adding 5 μL of proteinase K enzyme and incubating at 55 degrees C for 15 minutes. The beads were captured with a magnet and the supernatant was discarded. The beads were washed with 200 μL of bead binding buffer+TWEEN-20 then washed with 200 μL of 100 mM NaOH followed by one wash with 200 μL bead binding buffer+TWEEN-20 and one wash with 200 μL pre-ligation buffer.

USER cleavage from support. A USER reaction mix was prepared that included 0.10 μM USER enzyme in 1× rCutSmart buffer, commercially available from New England Biolabs. The beads from the second ligation reaction were collected, the supernatant was discarded and the beads were resuspended in 50 μL of the USER reaction mix. The sample was subjected to thermoshaking at 2000 rpm for 15 minutes at 37 degrees C. The beads were pelleted and the supernatant containing the cleaved and released duplex template constructs was collected and retained. The sample of nucleic acids was subjected to two rounds of SPRI clean up, as described above.

The presence of the desired duplex template construct in the sample of purified nucleic acids can be assessed using art-recognized techniques, such as visualization of nucleic acids of the expected size by gel analysis. The final sample of duplex template constructs may be quantitated using molecular beacon-based technologies. For example, a hairpin probe can be designed with a molecular beacon joined to the end of one strand in the stem portion of the adapter that is quenched by proximity to a quenching moiety joined to the end of the other strand of the hairpin stem. The sequence of the quenching strand of the hairpin stem can also be designed to hybridize to a single stranded arm of the Y adapter of the duplex template construct. When the hairpin probe hybridizes to the duplex template construct, the hairpin adapter linearizes and the molecular beacon signal at one end is liberated from the quenching effect of the quenching moiety at the other end and is thus capable of being detected.

### Example 2

### Synthesis of a “Parent-Parent” Duplex Template Construct in Solution

In this example, a parent-parent duplex template construct was synthesized in solution. The molar ratio of the hairpin adapter to target fragment insert DNA was 3:1 (3×) and the molar ratio of Y adapter to target fragment insert DNA was 10:1 (10×).

ERAT reaction. An ERAT reaction was prepared that included 50 ng of the double stranded library of DNA target fragments (i.e., fragmented human genomic DNA), 7 μL of ERAT enzyme in 1×ERAT buffer, as described further in Example 1. The volume of the ERAT reaction was 60 μL. The ERAT reaction was cycled in a thermocycler for 30 minutes at 20 degrees C followed by 30 minutes at 65 degrees C.

YAD hybridization. A 20 μL YAD hybridization reaction was prepared that included 1 μM of each oligonucleotide strand of the YAD (i.e., the 5′ oligonucleotide strand and the 3′ oligonucleotide strand) in 100 mM NaCl and 20 mM TrisCl, pH 8.0. For this experiment, the 5′ oligonucleotide strand included the following features: a 32mer oligonucleotide sequence that forms the 5′ single stranded arm in the final Y adapter structure and includes an extension oligonucleotide hybridization sequence, a 16mer oligonucleotide sequence that forms the double stranded stem portion in the final Y adapter structure, and a terminal 3′ deoxythymidine. The 3′ oligonucleotide strand included the following features: a 16mer oligonucleotide sequence that forms that double stranded stem portion in the final Y adapter structure and a 52mer oligonucleotide sequence that forms the 3′ single stranded arm in the final Y adapter structure and includes a cleavage site that is specifically recognized and cleaved by the Nt.BstNB1 nickase enzyme. The reaction was cycled in a thermocycler for one minute at 95 degrees C, one minute at 75 degrees C, and one minute at 55 degrees C. The resulting sample of hybridized YAD was stored a four degrees C.

First and Second Ligations. A ligation master mix was prepared that included 0.27 U/μL of PNK enzyme, 1.36 U/μL of deadenylase enzyme, and 0.09 U/μL of Codexis DNA ligase in HyperPrep ligation buffer. A first ligation reaction was prepared that included 0.5 ng/μL of ERAT insert DNA (i.e., the end-repaired and A-tailed DNA target fragment), and 0.01 μM of PNK hairpin adapter (prepared as described in Example 1) in 1× ligation master mix. The 100 μL ligation reaction was incubated at four degrees C for one hour.

The first ligation reaction was denatured by adding 60.45 μL of water and heating the sample to 95 degrees C for four minutes. The second ligation reaction was prepared by adding 0.02 μM of hybridized YAD and 35.49 μL of 1× ligation master mix to the denatured first ligation reaction and incubating at four degrees C for one hour. The final volume of the second ligation reaction was 200 μL. The second ligation reaction was subjected to two rounds of SPRI clean-up, as described in Example 1.

Exonuclease treatment. A 40 μL sample including the duplex template construct product of the second ligation reaction was subjected to exonuclease-mediated digestion to remove single stranded DNA side-products. 4.5 μL of 10× IsoAmp II buffer and 1 μL of Lambda Exonuclease was added to the sample at incubated at 37 degrees C for 30 minutes. The sample was then subjected to two rounds of SPRI clean-up, as described in Example 1.

### Example 3

Rapid Whole Genome Sequencing (rWGS) with “SBX Fast”

It is well established that genetic diseases are a leading cause of pediatric deaths. Unfortunately, most currently available WGS technologies can take up to several weeks to produce a diagnosis. Thus, there is a need in the art for more rapid diagnosis of genetic diseases to improve prognosis in critical patient populations.

To address this, and other, unmet medical needs, this Example describes adaptation of the SBX® methodology to reduce time-to-result. Exemplary adaptations include optimization and/or omission of certain steps of the overall workflow. For example, the library preparation workflow described in Example 2 has been streamlined into a workflow referred to as “SBX Fast” for applications requiring a rapid turnaround time, such as genomic analysis of high-risk newborn infants in a Neonatal Intensive Care Unit (NICU) clinical setting.

In this application, a blood sample of around 50 μL to 100 μL (or more, depending on the urgency) is collected from a NICU patient. Empirically, a blood sample of 50 μL is expected to yield around 2 μg of genomic DNA, while a sample of 100 μL is expected to yield around 4 μg of genomic DNA. In the former case, 2 μg of genomic DNA processed through the present library preparation workflow is expected to yield around 1.0 to 1.5 pmol gDNA library, which is sufficient for Xpandomer synthesis and nanopore sequencing. In certain situations, samples of genomic DNA may also be obtained from one or more parents of the patient or from unrelated individuals for the purpose of generating non-patient genomic DNA libraries in parallel to optimize downstream Xpandomer synthesis steps. Each individual library is distinguished by a unique SID identifier provided by the hairpin adapter of the library constructs. For example, a “proband” library (patient only) may be pooled with an unrelated, premade library that serves as “buffer” DNA during Xpandomer synthesis and sequencing. Alternatively, a “duo” library (one parent) or “trio” library (both parents) may be prepared in which library constructs derived from patient gDNA are pooled with library constructs derived from one or both of parental gDNA. The duo and trio libraries are expected to assist in sequence analysis, e.g., to expedite identification of disease causing variants during tertiary analysis. Advantageously, the SBX Fast workflow does not require amplification of the DNA library. Not only does this reduce the overall time of library preparation, it also reduces the likelihood of propagating PCR-induced artificial mutations into the library fragments.

The inventors have further determined that certain time-consuming steps may be omitted from the library preparation workflow without negatively impacting library quality. For example, the wash steps that typically follow size-selective purification (e.g., the SPRI clean-up steps) may be omitted from the workflow. In addition, it has been observed that the time allocated for various steps or reactions may be reduced. For example, the incubation time of the first ligation reaction may be reduced; in certain exemplary ligation conditions, the time may be reduced to around 10 minutes when the sample is incubated at around 23 degrees C. Likewise, the incubation time of the second ligation reaction may be reduced, e.g., to from around 15 to around 20 minutes when the sample is incubated at around 20 degrees C. In other workflows, the first and second ligations are pooled into a “one-pot” ligation reaction.

Patient-derived and non-patient-derived libraries may be, in certain instances, pooled for preparation of a sample of Xpandomer molecules for nanopore sequence determination. For example, libraries may be pooled into a single Xpandomer synthesis reaction and/or a single Xpandomer processing reaction in order to drive the relevant reaction(s) to completion and/or maximize recovery of mature Xpandomer molecules for passage through a nanopore sensor. Advantageously, the target run time for rWGS under SBX Fast conditions is around 6-7 hours or less, while still yielding an expected throughput of around 150 Gb of sequence data, demonstrating over 40× genome coverage with a quality score of over Q40.

### SPECIFICALLY INCLUDED EMBODIMENTS

The following embodiments are specifically contemplated as part of the disclosure. This is not intended to be an exhaustive listing of potentially claimed embodiments included within the scope of the disclosure.

Embodiment 1. A method of producing a duplex nucleic acid template, the method comprising the steps of:
(a) providing a double stranded nucleic acid target fragment comprising a first end and a second end;
(b) ligating a first adapter to the first end of the double stranded nucleic acid target fragment to produce a sample of first ligation products, wherein the first adapter is a hairpin adapter, and wherein the strands of the double stranded nucleic acid target fragment are covalently joined by the hairpin adapter;
(c) treating the sample of first ligation products with denaturation conditions; and
(d) ligating a second adapter to the second end of the double stranded nucleic acid target fragment to produce a sample of second ligation products, wherein the second adapter is a Y adapter.

Embodiment 2. The method of embodiment 1, further comprising a step of treating the sample of second ligation products of step (d) with an exonuclease enzyme.

Embodiment 3. The method of embodiment 1, further comprising a step of purifying the second ligation products of step (d).

Embodiment 4. The method of any one of embodiments 1 to 3, wherein the molar ratio of the hairpin adapter to the double stranded nucleic acid target fragment is around 3:1 or more.

Embodiment 5. The method of any one of embodiments 1 to 4, wherein the molar ratio of the Y adapter to the double stranded nucleic acid target fragment is from around 3:1 to around 10:1 or less.

Embodiment 6. The method of any one of embodiments 1 to 5, wherein the hairpin adapter comprises a SID.

Embodiment 7. The method of any one of embodiments 1 to 6, wherein the Y adapter comprises a hybridization site for an extension oligonucleotide.

Embodiment 8. The method of any one of embodiments 1 to 7, wherein the denaturing conditions comprise heat treatment.

Embodiment 9. The method of embodiment 3, wherein the step of purifying the second ligation products from the sample of ligation products of step (d) comprises contacting the sample of second ligation products with solid-phase reversable immobilization (SPRI) beads.

Embodiment 10. The method of any one of embodiments 1 to 9, wherein the step of ligating a first adapter to the first end of the double stranded nucleic acid target fragment comprises incubating the sample for 30 minutes or less under DNA ligation conditions.

Embodiment 11. The method of any one of embodiments 1 to 10, wherein the step of ligating a second adapter to the second end of the double stranded nucleic acid target fragment comprises incubating the sample for around 30 minutes or less under DNA ligation conditions.

Embodiment 12. The method of any one of embodiments 1 to 11, wherein the Y adapter is bound to a solid support.

Embodiment 13. The method of embodiment 12, wherein the binding of the Y adapter to the solid support is mediated by a cleavable linker.

Embodiment 14. The method of embodiment 12, wherein the solid support comprises streptavidin-coated magnetic beads.

Embodiment 15. The method of any one of embodiments 1 to 14, wherein step (b) further comprises ligating the first adapter to the second end of the double stranded nucleic acid target fragment to produce the sample of first ligation products.

Embodiment 16. The method of embodiment 15, further comprising the step of fragmenting the sample of first ligation products to produce a sample of fragmentation products, wherein the fragmentation products comprise a first end comprising the hairpin adapter and a second end lacking the hairpin adapter prior to step (d).

Embodiment 17. The method of any one of embodiments 1 to 16, wherein step (d) is combined with step (a) in the same reaction vessel.

Embodiment 18. The method of embodiment 17, wherein the ratio of the Y adapter to the double stranded nucleic acid target fragment is 3:1 or more.

Embodiment 19. A method of producing a duplex nucleic acid template, the method comprising the steps of: (a) providing a double stranded nucleic acid target fragment comprising a first adapter at a first end and a second adapter at a second end, wherein each strand of the double stranded nucleic acid target fragment comprises a single stranded break providing a free 3′ end; (b) contacting the double-stranded nucleic acid target fragment with a nucleic acid polymerase under nucleic acid synthesis conditions, wherein the nucleic acid polymerase initiates template-dependent synthesis from the free 3′ ends to produce a first and a second nucleic acid synthesis product, wherein the first and second nucleic acid synthesis products each comprise a newly synthesized daughter strand and a free end; and (c) ligating a third adapter to the first and the second nucleic acid synthesis products, wherein the third adapter is ligated to the free end of the first and the second nucleic acid synthesis product.

Embodiment 20. The method of embodiment 19, wherein the first adapter and the second adapter are Y adapters.

Embodiment 21. The method of embodiment 20, wherein the free 3′ ends are provided by partial ligation of the first and the second Y adapters to the double stranded nucleic acid target fragment.

Embodiment 22. The method of embodiment 20 or 21, wherein the third adapter is a hairpin adapter.

Embodiment 23. The method of embodiment 19, wherein the first adapter and the second adapter are hairpin adapters, wherein the hairpin adapters comprise a double stranded stem portion and a single stranded loop portion.

Embodiment 24. The method of embodiment 23, wherein the free 3′ ends are provided by a single stranded break in one strand of the double stranded stem portion of the hairpin adapters.

Embodiment 25. The method of embodiment 23 or 24, wherein the third adapter is a Y adapter.

Embodiment 26. The method of any one of embodiments 19 to 25, wherein the nucleic acid synthesis conditions comprise nucleotide analogs, wherein the nucleotide analogs form weaker hydrogen bonds with complementary nucleotides relative to native nucleotides.

Embodiment 27. The method of embodiment 26, wherein the nucleotide analogs comprise N4-Me dCTP and 7-deaza dGTP.

Embodiment 28. The method of any one of embodiments 19 to 27, wherein at least one of the adapters comprises one or more features selected from the group consisting of a UMI, a SID, an extension oligonucleotide hybridization stie, and a blocking oligonucleotide hybridization site.

Embodiment 29. The method of any one of embodiments 19 to 28, wherein the nucleic acid polymerase comprises a strand displacing DNA polymerase.

Embodiment 30. The method of embodiment 29, wherein the strand displacing DNA polymerase is selected from the group consisting of Klenow fragment, BST large fragment DNA polymerase, and Phi29 DNA polymerase, or variants thereof.

Embodiment 31. The method of any one of embodiments 19 to 28, wherein the nucleic acid polymerase comprises DPO4 DNA polymerase or a variant thereof.

Embodiment 32. The method of any one of embodiments 19 to 31, wherein the duplex nucleic acid template is produced on a solid support.

Embodiment 33. The method of any one of embodiments 19 to 31, wherein the duplex nucleic acid template is produced in solution.

Embodiment 34. The method of any one of embodiments 19 to 33, wherein the duplex nucleic acid template is treated with an agent that selectively converts native cytosine residues to uracil residues.

Embodiment 35. The method of any one of embodiments 19 to 33, wherein the duplex nucleic acid template is treated with a DNA glycosylase enzyme and an aminoxyalkyl uracil mimetic compound, wherein the DNA glycosylase enzyme and the aminoxyalkyl uracil mimetic compound selectively convert epigenetically modified cytosine residues to uracil oxime mimetic residues.

Embodiment 36. A method of producing an opened duplex nucleic acid template, comprising the steps of: (a) providing a duplex nucleic acid template according to any one of claims 1 to 35; (b) contacting the duplex template construct with an oligonucleotide primer, wherein the oligonucleotide primer hybridizes to the hairpin adapter; (c) contacting the oligonucleotide primer with a DNA polymerase under DNA synthesis conditions; and (d) extending the oligonucleotide primer to produce a complementary copy of a first strand of the duplex nucleic acid template and displace a second strand of the duplex nucleic acid template, wherein the displaced second strand of the duplex nucleic acid template provides a single stranded template.

Embodiment 37. A method of producing an Xpandomer copy of a nucleic acid template, comprising the steps of: (a) providing a duplex nucleic acid template according to any one of claims 1 to 36; and (b) contacting the duplex nucleic acid template with a modified nucleic acid polymerase under Xpandomer synthesis conditions, wherein the Xpandomer copy of the nucleic acid template comprises two copies of the nucleic acid target fragment.

Embodiment 38. The method of embodiment 37, wherein the Xpandomer synthesis conditions comprise one or more of an extension oligonucleotide, a buffer/salt system, a polymerase cofactor, a polymerase enhancing moiety (PEM), XNTP substrates, a phosphate shield molecule, a solvent, a crowding agent, and a blocking oligonucleotide.

Embodiment 39. The method of embodiment 37 or 38, wherein the modified nucleic acid polymerase is a variant of DPO4 DNA polymerase (SEQ ID NO:1).

Embodiment 40. The method of embodiment 39, wherein the variant of DPO4 DNA polymerase comprises an amino acid sequence that is at least 85% identical to SEQ ID NO:2.

Embodiment 41. The method of embodiment 38, wherein the Xpandomer synthesis conditions comprise an additive as set forth in Table 3.

Embodiment 42. The method of embodiment 38, wherein the additive comprises a single stranded binding protein (SSB) selected from the group consisting of TTH (SEQ ID NO:3), KOD (SEQ ID NO:4), Gp32 (SEQ ID NO:5), E. coli SSB (SEQ ID NO:6), RPA (SEQ ID NO:7), NCp7 (SEQ ID NO:8), RecA (SEQ ID NO:9), and helicase (SEQ ID NO:10).

Embodiment 43. The method of embodiment 38, wherein Xpandomer synthesis conditions comprise an additive selected from the group consisting of 4,4-(furan-2,5-diylbis(1H-1,2,3-triazole-4,1-diyl))bis(2-(trifluoromethyl)benzoic acid), 4,4′-((4-ethylcarbamoyl)pyridine-2,6-diyl)bis(1H-1,2,3-triazole-4,1-diyl))bis(2-trifluoromethyl)benzoic acid), p-{4-[4-(Ethylaminosulfonyl)-6-[1-(p-sulfophenyl)-1H-1,2,3-triazol-4-yl]-2-pyridyl]-1H-1,2,3-triazol-1-yl}benzenesulfonic acid, Acetamide, and NMP:DMSO.

Embodiment 44. The method of embodiment 38, wherein Xpandomer synthesis conditions comprise applying a stretching force selected from the group consisting of brownian motion, hydrodynamic force, magnetic force, acoustic force, electrophoretic force, electroosmotic force, and photonic force.

Embodiment 45. A method of sequencing a plurality of duplex nucleic acid templates, comprising the steps of: (a) providing a sample comprising a plurality of duplex nucleic acid templates, wherein the plurality of duplex nucleic acid templates each comprise a sense strand of a nucleic acid target fragment covalently joined to an antisense strand of a nucleic acid target fragment; (b) generating a copy of the each of the plurality of duplex nucleic acid templates, wherein the copies comprise an Xpandomer, wherein the Xpandomers comprise a sequence of reporter codes, and wherein the sequence of reporter codes encodes the sequence of the duplex nucleic acid templates; and (c) determining the sequences of the reporter codes by passing the Xpandomers through a nanopore sensor.

Embodiment 46. The method of embodiment 45, wherein the duplex nucleic acid templates comprise the duplex nucleic acid templates according to any one of embodiments 1 to 36.

Embodiment 47. The method of embodiment 45 or 46, wherein the nucleic acid target fragments are isolated from a blood sample.

Embodiment 48. The method of embodiment 47, wherein the time to complete the method is from six to seven hours or less.

Embodiment 49. A composition comprising the duplex nucleic acid template according to any one of embodiments 1 to 36.

Embodiment 50. A kit for producing a library of duplex nucleic acid templates, comprising one or more of: a nucleic acid fragmentation mixture, a first ligation mixture, wherein the first ligation mixture comprises a hairpin adapter, wherein the hairpin adapter comprises a SID (HPS), a second ligation mixture, wherein the second ligation mixture comprises a Y adapter, an exonuclease digest mixture, a nucleic acid purification mixture, and a nucleic acid quantification assay mixture.

Embodiment 51. A method of producing a YSU adapter comprising the steps of: (a) providing a first sample comprising a first YSU adapter portion comprising a 5′ oligonucleotide single stranded arm, a 3′ oligonucleotide single stranded arm and a first double stranded stem portion, wherein the first double stranded stem portion comprises a SID sequence; (b) providing a second sample comprising a second YSU adapter portion, comprising a second double stranded stem portion, wherein the second double stranded stem portion comprises a UMI sequence; and (c) contacting the first sample and the second sample with a DNA ligase under DNA ligation conditions to produce a YSU adapter comprising a SID sequence and a UMI sequence.

Embodiment 52. The method of embodiment 51, wherein the second sample comprises a plurality of second YSU adapter portions, wherein each of the plurality of second YSU adapter portions comprises a unique UMI sequence.

Embodiment 53. A composition comprising the YSU adapter according to embodiment 51 or 52.

What is claimed is:

1. A method of producing a duplex nucleic acid template, the method comprising the steps of:
(a) providing a double stranded nucleic acid target fragment comprising a first end and a second end;
(b) ligating a first adapter to the first end of the double stranded nucleic acid target fragment to produce a sample of first ligation products, wherein the first adapter is a hairpin adapter, and wherein the strands of the double stranded nucleic acid target fragment are covalently joined by the hairpin adapter;
(c) treating the sample of first ligation products with denaturation conditions; and
(d) ligating a second adapter to the second end of the double stranded nucleic acid target fragment to produce a sample of second ligation products, wherein the second adapter is a Y adapter.

2. The method of claim 1, further comprising a step of treating the sample of second ligation products of step (d) with an exonuclease enzyme.

3. The method of claim 1, further comprising a step of purifying the second ligation products of step (d).

4. The method of claim 1, wherein the molar ratio of the hairpin adapter to the double stranded nucleic acid target fragment is around 3:1 or more.

5. The method of claim 1, wherein the molar ratio of the Y adapter to the double stranded nucleic acid target fragment is from around 3:1 to around 10:1 or less.

6. The method of claim 1, wherein the hairpin adapter comprises a SID.

7. The method of claim 1, wherein the Y adapter comprises a hybridization site for an extension oligonucleotide.

8. The method of claim 1, wherein the denaturing conditions comprise heat treatment.

9. The method of claim 3, wherein the step of purifying the second ligation products from the sample of ligation products of step (d) comprises contacting the sample of second ligation products with solid-phase reversable immobilization (SPRI) beads.

10. The method of claim 1, wherein the step of ligating a first adapter to the first end of the double stranded nucleic acid target fragment comprises incubating the sample for 30 minutes or less under DNA ligation conditions.

11. The method of claim 1, wherein the step of ligating a second adapter to the second end of the double stranded nucleic acid target fragment comprises incubating the sample for around 30 minutes or less under DNA ligation conditions.

12. The method of claim 1, wherein the Y adapter is bound to a solid support.

13. The method of claim 12, wherein the binding of the Y adapter to the solid support is mediated by a cleavable linker.

14. The method of claim 12, wherein the solid support comprises streptavidin-coated magnetic beads.

15. The method of claim 1, wherein step (b) further comprises ligating the first adapter to the second end of the double stranded nucleic acid target fragment to produce the sample of first ligation products.

16. The method of claim 15, further comprising the step of fragmenting the sample of first ligation products to produce a sample of fragmentation products, wherein the fragmentation products comprise a first end comprising the hairpin adapter and a second end lacking the hairpin adapter prior to step (d).

17. The method of claim 1, wherein step (d) is combined with step (a) in the same reaction vessel.

18. The method of claim 17, wherein the ratio of the Y adapter to the double stranded nucleic acid target fragment is 3:1 or more.

19. A method of producing a duplex nucleic acid template, the method comprising the steps of:
(a) providing a double stranded nucleic acid target fragment comprising a first adapter at a first end and a second adapter at a second end, wherein each strand of the double stranded nucleic acid target fragment comprises a single stranded break providing a free 3′ end;
(b) contacting the double-stranded nucleic acid target fragment with a nucleic acid polymerase under nucleic acid synthesis conditions, wherein the nucleic acid polymerase initiates template-dependent synthesis from the free 3′ ends to produce a first and a second nucleic acid synthesis product, wherein the first and second nucleic acid synthesis products each comprise a newly synthesized daughter strand and a free end; and
(c) ligating a third adapter to the first and the second nucleic acid synthesis products, wherein the third adapter is ligated to the free end of the first and the second nucleic acid synthesis product.

20. The method of claim 19, wherein the first adapter and the second adapter are Y adapters, wherein the free 3′ ends are provided by partial ligation of the first and second Y adapters to the double stranded nucleic acid target fragment, and wherein the third adapter is a hairpin adapter.


SEQUENCE LISTING (10 sequences)

SEQ ID NO: 1 - protein, 352 aa, Sulfolobus solfataricus
MIVLFVDFDYFYAQVEEVLNPSLKGKPVVVCVFSGRFEDSGAVATANYEARKFGVKAGIPIVEAKKILPNAVYLPMRKEVYQQVSSRIMNLLREYSEKIEIASIDEAYLDISDKVRDYREAYNLGLEIKNKILEKEKITVTVGISKNKVFAKIAADMAKPNGIKVIDDEEVKRLIRELDIADVPGIGNITAEKLKKLGINKLVDTLSIEFDKLKGMIGEAKAKYLISLARDEYNEPIRTRVRKSIGRIVTMKRNSRNLEEIKPYLFRAIEESYYKLDKRIPKAIHVVAVTEDLDIVSRGRTFPHGISKETAYSESVKLLQKILEEDERKIRRIGVRFSKFIEAIGLDKFFDT

SEQ ID NO: 2 - protein, 340 aa, Sulfolobus solfataricus
MIVLFVDFDYFYAQVEEVLNPSLKGKPVVVCVFSGRTELSGAVATANYEARKFGVYSGMPIVRAKKILPNAVYLPWREPVYWGVSERIMNLLREYSEKIEIASIDEAYLDISDKVRDYREAYNLGLEIKNKILEKEKITVTVGISKNKVFAAVAGSKAKPNGIKVIDDEEVKRLIRELNIADVQGIPYFTAQKLKKLGINKLVDTLSIEFDKLKGMIGEAKAKYLISLARDEYNEPIRTRVRKSIGRTVTMKRNSRNLEEIKPYLFRAIEECYYKLDKRIPKAIHVVAWRSRWNSQYRWSWFPHGISKETAYSESVKLLQQILKKDKRKIRRIGVRFSKF

SEQ ID NO: 3 - protein, 263 aa, Thermus thermophilus
MARGLNRVFLIGALATRPDMRYTPAGLAILDLTLAGQDLLLSDNGGEREVSWYHRVRLLGRQAEMWGDLLDQGQLVFVEGRLEYRQWEREGEKRSELQIRADFLDPLDDRGKERAEDSRGQPRLRAALNQVFLMGNLTRDPELRYTPQGTAVARLGLAVNERRQGAEERTHFVEVQAWRDLAEWAAELRKGDGLFVIGRLVNDSWTSSSGERRFQTRVEALRLERPTRGPAQAGGSRSREVQTGGVDIDEGLEDFPPEEELPF

SEQ ID NO: 4 - protein, 356 aa, Thermococcus kodakarensis
MEVLTKDEIINRIIRERGLSRSEIEEKIRELAKMHGVSENAAAVMLAEELGVSLGKEEEMLYIKDLVPGMTGVNIVARIKRKFPPREYTRRDGSTGRVADLIIYDSTGQARLVLWDAMVAKYYDDLNVGDVIKVIDPTVKEGMRGVELHANFRTRIIKNPEDPRVEEIPPLEEVRSYNYRRVQIKELQGGERFVEVRGTIAKLYRVLVYDACPECRRRVDYDPSTDTWICPEHGPVNPVKITVLDFGLDDSTGYIRTTLFGDSAAELIGEEPEVIDEKLKKLIDEGLTPKEAGKRLAEDEYYPLIGKEIVVRGSVVEDKFLGTLFKARSWDEVNEKAEIERVRRELYRELKEYGLE

SEQ ID NO: 5 - protein, 301 aa, Bacteriophage T4
MFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDL

SEQ ID NO: 6 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQSGWGQPQQPQGGNQFSGGAQSRPQQSAPAAPSNEPPMDFDDDIPF

SEQ ID NO: 7 - protein, 616 aa, Homo sapiens
MVGQLSEGAIAAIMQKGDTNIKPILQVINIRPITTGNSPPRYRLLMSDGLNTLSSFMLATQLNPLVEEEQLSSNCVCQIHRFIVNTLKDGRRVVILMELEVLKSAEAVGVKIGNPVPYNEGLGQPQVAPPAPAASPAASSRPQPQNGSSGMGSTVSKAYGASKTFGKAAGPSLSHTSGGTQSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCEDDHHLPTVQFDFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQALDGVSISDLKSGGVGGSNTNWKTLYEVKSENLGQGDKPDYFSSVATVVYLRKENCMYQACPTQDCNKKVIDQQNGLYRCEKCDTEFPNFKYRMILSVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 8 - protein, 71 aa, Human immunodeficiency virus 1
MQRGNFRNQRKMVKCFNCGKEGHTARNCRAPRKKGCWKCGKEGHQMKDCTERQANFLGKIWPSYKGRPGNF

SEQ ID NO: 9 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 10 - protein, 720 aa, Escherichia coli
MDVSYLLDSLNDKQREAVAAPRSNLLVLAGAGSGKTRVLVHRIAWLMSVENCSPYSIMAVTFTNKAAAEMRHRIGQLMGTSQGGMWVGTFHGLAHRLLRAHHMDANLPQDFQILDSEDQLRLLKRLIKAMNLDEKQWPPRQAMWYINSQKDEGLRPHHIQSYGNPVEQTWQKVYQAYQEACDRAGLVDFAELLLRAHELWLNKPHILQHYRERFTNILVDEFQDTNNIQYAWIRLLAGDTGKVMIVGDDDQSIYGWRGAQVENIQRFLNDFPGAETIRLEQNYRSTSNILSAANALIENNNGRLGKKLWTDGADGEPISLYCAFNELDEARFVVNRIKTWQDNGGALAECAILYRSNAQSRVLEEALLQASMPYRIYGGMRFFERQEIKDALSYLRLIANRNDDAAFERVVNTPTRGIGDRTLDVVRQTSRDRQLTLWQACRELLQEKALAGRAASALQRFMELIDALAQETADMPLHVQTDRVIKDSGLRTMYEQEKGEKGQTRIENLEELVTATRQFSYNEEDEDLMPLQAFLSHAALEAGEGQADTWQDAVQLMTLHSAKGLEFPQVFIVGMEEGMFPSQMSLDEGGRLEEERRLAYVGVTRAMQKLTLTYAETRRLYGKEVYHRPSRFIGELPEECVEEVRLRATVSRPVSHQRMGTPMVENDSGYKLGQRVRHAKFGEGTIVNMEGSGEHSRLQVAFQGQGIKWLVAAYARLESV